(19) United States
(12) Patent Application Publication
(10) Pub. No.: US 2024/0384279 A1
(43) Pub. Date: Nov. 21, 2024

(54) MICROBIOTA ENGINEERING

(71) Applicant: SNIPR BIOME APS, Copenhagen Ø (DK)

(72) Inventors: Christian MUNCK, Copenhagen (DK); Andreas PORSE, Copenhagen (DK); Jakob KRAUSE HAABER, Copenhagen (DK); Lev KOVAL, Copenhagen (DK); Lauge ALFASTSEN, Copenhagen (DK); Szabolcs SEMSEY, Copenhagen (DK)

(21) Appl. No.: 18/294,101

(22) PCT Filed: Aug. 1, 2022
(86) PCT No.: PCT/EP2022/071562

§ 371 Date: Jan. 31, 2024

(30) Foreign Application Priority Data
Aug. 1, 2021 (GB) 2111068.9
Nov. 14, 2021 (GB) 2116398.5

(51) Int. Cl. C12N 15/70; A61K 38/00; A61K 48/00; A61P 31/04; C12N 9/22; C12N 15/11; C12N 15/74

(52) U.S. Cl. CPC C12N 15/70; A61K 48/0066; A61P 31/04; C12N 9/22; C12N 15/11; C12N 15/74; A61K 38/00; C12N 2310/20

(57) ABSTRACT

The invention relates to methods and means for temporally regulating the production of products of interest (eg, proteins or RNAs) in microbiota of subjects, such as gut microbiota of humans or animals.

### CROSS REFERENCE TO RELATED APPLICATIONS

This application is a U.S. national stage application under 35 U.S.C. § 371 of International Application No. PCT/EP2022/071562, filed internationally on Aug. 1, 2022, which claims priority to GB Application No. 2111068.9, filed Aug. 1, 2021, and GB Application No. 2116398.5. filed Nov. 14, 2021, the disclosures of which are herein incorporated by reference in their entirety.

### REFERENCE TO AN ELECTRONIC SEQUENCE LISTING

The contents of the electronic sequence listing (786212001600SEQLIST.xml; Size: 18,622 bytes; and Date of Creation: Jan. 24, 2024) is herein incorporated by reference in its entirety.

### TECHNICAL FIELD

The invention relates to methods and means for temporally regulating the production of products of interest (eg, proteins or RNAs) in microbiota of subjects. such as gut microbiota of humans or animals. For example, in this way microbiota can usefully be modified for expression of products in a controlled and optionally reversible manner.

### BACKGROUND

Mounting evidence undoubtedly links homeostasis in human microbiotas to human health and conversely, microbial dysbiosis is linked to a variety of disease conditions. For example, dysbiosis such as overgrowth of the bacterial pathogen C. difficile in the gut microbiota is linked to severe diarrhoea caused by production of toxins secreted from the C. difficile. Another example of disease related to microbial dysbiosis is inflammatory bowel disease (TBD), in which an inflammatory state of the intestines is at least partly caused by the metabolome associated with overgrowth of Enterobacteriaceae and under representation of bacterial species capable of producing short-chain fatty acids.

Reverting a disease condition by restoring a healthy microbiota by faecal microbial transplants (FMT), in which the faecal microbiota from a healthy donor is transplanted into the gut of the patient suffering from microbial dysbiosis has been proven effective to treat for example recurrent C. difficile infections. However, FMT is a relatively uncontrolled process and also associated with risks as demonstrated by fatalities caused by bacterial infections that could be traced back to a FMT. A more controlled approach in which single strains or consortia of defined strains are added to the dysbiotic gut environment is currently being heavily investigated. Such approaches require the displacement of strains by colonizing new strains added in the consortia

In some cases, target compounds of interest are identified, such as the presence of bacterial toxins (against which antibodies have been generated) or the absence of IL-22 associated with disease. The delivery, however, of relevant proteinaceous compounds locally in the intestinal tract is inefficient and cumbersome due to instability of the proteins in the gut environment.

### SUMMARY OF THE INVENTION

Hence, local in situ production of relevant molecules (e.g. enzymes, antibodies, nanobodies, small molecules) intracellularly or secreted by live bacteria which are established and already present in the microbiota would allow targeted intervention with minimal impact on the native microbiota. The invention addresses this need and how to control such production in situ in microbiota.

The invention thus provides the following configurations:—

### In a First Configuration

At least one nucleic acid vector for transfer into a host cell of a microbiota. the vector(s) comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in
the host cell, wherein P2 is operable in the host cell to regulate expression or activity of P1; wherein
(c) NS1 is under the control of a first promoter (eg, a constitutive or inducible promoter) for expression of P1; and
(d) expression or activity of P2 in the host cell is regulatable by exposure of the host cell comprising the at least one vector to a regulator agent (R), thereby regulating the expression or activity of P1.

In an embodiment there is provided:—

At least one nucleic acid vector for transfer into a host cell of a microbiota, the vector(s) comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to regulate expression of P1; wherein
(c) NS1 is under the control of a first promoter (eg, a constitutive or inducible promoter) for expression of P1; and
(d) expression of P2 in the host cell is regulatable by exposure of the host cell comprising the at least one vector to a regulator agent (R), thereby regulating the expression P1.

At least one nucleic acid vector for transfer into a host cell of a microbiota, the vector(s) comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to regulate expression of P1;
   wherein
(c) NS1 is under the control of a first promoter (eg, a constitutive or inducible promoter) for expression of Pt; and
(d) NS2 is under the control of a second promoter that is regulatable for expression of P2, wherein binding of a regulator agent (R) to the vector nucleic acid regulates the second promoter, thereby regulating the expression of P2 and P1.

Preferably, NS1 and NS2 are comprised by the same nucleic acid vector. In another configuration, exposure of a regulator agent (R) to the vector nucleic acid regulates the second promoter, thereby regulating the expression of P2 and P1.

Preferably, P2 is operable in the host cell to bind to vector nucleic acid to regulate expression of P1.

In an embodiment, the invention provides a host cell comprising said at least one vector.

In an embodiment, P1 is a protein (eg, an enzyme) of a metabolic pathway in the host cell, wherein said activity is an activity (eg, enzymatic activity) of P1 in the pathway. In an example, P1 is a protein inhibitor and the activity is inhibitor activity (eg, inhibitor of a component of a metabolic pathway). In an example, Pt is a binding agent, eg, an antibody or antibody fragment, such as a single domain antibody (eg, a nanobody) or an scFv.

In an embodiment, P2 is an inhibitor of P1 expression. In an embodiment, P2 activity is inhibition of P1 expression. In an embodiment, P2 is an enhancer of P1 expression. In an embodiment, P2 activity is enhancement of P1 expression.

### In a First Aspect of the First Configuration

A nucleic acid vector for transfer into a host cell of a microbiota, the vector comprising a nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to bind to the vector nucleic acid to regulate expression of P1;
   wherein
(c) NS1 is under the control of a first promoter (eg, a constitutive or inducible promoter) for expression of P1; and
(d) NS2 is under the control of a second promoter that is regulatable for expression of P2, wherein binding of a regulator agent (R) to the vector nucleic acid regulates the second promoter, thereby regulating the expression of P2 and P1.

### In a Second Aspect of the First Configuration

P2 is expressible in the host cell for the formation of a nuclease that is operable in the host cell to cut the vector nucleic acid, wherein the nucleic acid is degraded, thereby downregulating the expression of P1. This is useful to control the expression of P1 temporally (ie, for a predetermined window of time) in a subject, such as in a human or animal microbiota. For example, in this way microbiota can usefully be modified for expression of P1 in a controlled and optionally reversible manner.

### In a Third Aspect of the First Configuration

Each vector (such as according to the First Aspect) is a conjugative plasmid. This is useful to enable spreading of the vector nucleic acid—and thus expression of P1—within a targeted microbiota. When coupled with the First Aspect of the First Configuration, there is provided a powerful way to controllably modify the extent and timing of P1 expression in the microbiota.

### In a Fourth Aspect of the First Configuration

The host cell is a cell of commensal or probiotic bacterial cell species of a human or animal microbiota, preferably a Bacteroides species. Such species are present and maintained in native microbiota of humans and animals and thus, the invention provides means for relatively stable and controllable microbiota modification for temporally regulating the P1 expression.

### In a Second Configuration

A method of temporally regulating the production of an expression product in a human or animal subject, the method comprising
a) administering to a microbiota (eg, a gut microbiota) of the subject said at least one vector comprising nucleic acid, wherein the microbiota comprises a host cell (eg, bacterial cell) and the nucleic acid encodes a product of interest (P1); optionally wherein the administering is oral or topical administration;
b) allowing transfer of the nucleic acid into the host cell comprised by the microbiota and expression of P1 in the host cell; and
c) after step (b) exposing the microbiota to a regulator agent (R) that regulates the expression or activity of a regulator product (P2) in the host cell wherein P2 is operable in the host cell to regulate expression or activity of P1, optionally wherein R upregulates production of an RNA-guided nuclease/guide RNA complex in the host cell that is capable of targeting a protospacer comprised by the nucleic acid, wherein the nuclease cuts the nucleic acid and expression of P1 is rendered non-functional (eg, by degradation of the cut nucleic acid in the cell), wherein the nuclease (or a component thereof) and/or RNA (or a component thereof) is encoded by the nucleic acid.

### In a First Aspect of the Second Configuration

A method of temporally regulating the production of an expression product in a human or animal subject, the method comprising
a) administering to a microbiota (eg, a gut microbiota) of the subject a said vector of the First Aspect of the First Configuration, wherein the microbiota comprises a host cell (eg, bacterial cell) and the nucleic acid encodes a product of interest (P1); optionally wherein the administering is oral or topical administration.
b) allowing transfer of the nucleic acid into the host cell comprised by the microbiota and expression of P1 in the host cell; and
c) after step (b) exposing the microbiota to a regulator agent (R) that regulates the expression or activity of P2, optionally wherein R upregulates production of an RNA-guided nuclease/guide RNA complex in the host cell that is capable of targeting a protospacer comprised by the nucleic acid, wherein the nuclease cuts the nucleic acid and expression of P1 is rendered non-functional (eg, by degradation of the cut nucleic acid in the cell). wherein the nuclease (or a component thereof) and/or RNA (or a component thereof) is encoded by the nucleic acid.

### In a Third Configuration

A method of temporally regulating the production of an expression product in a human or animal subject, the method comprising
(a) administering to a microbiota (eg, a gut microbiota) of the subject said at least one vector of the invention; optionally wherein the administering is oral or topical administration;
(b) allowing transfer of the nucleic acid into a host cell comprised by the microbiota and expression of P1 in the host cell; and
(c) after step (b) exposing the microbiota to R (eg, by administering R to the subject), wherein R regulates the second promoter, thereby regulating the expression of P2 and P1.

### In a First Aspect of the Second or Third Configuration

A method of
a) treating or preventing a disease or condition in a human or animal subject by temporally regulating the production of P1 according to the method of the Second or Third Configuration; or
b) modifying a microbiota (eg, a gut microbiota) of a human or animal subject by temporally regulating the production of P1 according to the method of the Second or Third Configuration.

### In a Fourth Configuration

A nucleic acid vector for transfer into a host cell of a microbiota, wherein the vector is comprised by a carrier cell (eg, a bacterial cell) and encodes
a) a nuclease (optionally an RNA-guided nuclease or restriction endonuclease) that is operable in the carrier cell to cut a chromosome or episome (which is not the vector of the invention) of the carrier cell, optionally wherein the chromosome or episome is degraded; and/or
b) an RNA that is operable in the carrier cell for guiding an RNA-guided nuclease or a precursor of such an RNA, wherein the RNA guides the nuclease to cut a chromosome or episome (which is not the vector) of the carrier cell, optionally wherein the chromosome or episome is degraded;
   wherein the vector comprises one or more regulatable promoters for regulating expression of the nuclease of (a) and/or the RNA or component of (b) in the carrier cell.

### In a Fifth Configuration

A method of engineering a microbiome, the method comprising contacting the microbiome with a plurality of vectors as described herein and optionally allowing transfer of said vector nucleic acid into target cells of the microbiota.

A modified microbiota obtained or obtainable by the method herein, optionally wherein the microbiota is comprised by a pharmaceutical composition for use as a medicament to treat a disease or condition in a human or animal subject.

### In a Sixth Configuration

A host cell comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to regulate expression or activity of P1; wherein
(c) NS1 is under the control of a first promoter (eg, a constitutive or inducible promoter) for expression of P1; and
(d) expression or activity of P2 in the host cell is regulatable by exposure of the host cell comprising the at least one vector to a regulator agent (R), thereby regulating the expression or activity of P1;
   wherein the host cell is a bacterial, archaeal or fungal cell.

### In a Seventh Configuration

### In a First Aspect

A cell (optionally according to any other Configuration), comprising a nucleic acid, wherein the nucleic acid comprises a gene encoding a product of interest (P1), the gene comprising a nucleotide sequence (NS1) encoding P1 and a regulatory region 5′ of NS1 that comprises a promoter (Px) for controlling the expression of NS1, wherein the combination of Px and NS1 is heterologous to the cell and Px is regulatable by xylitol.

### In a Second Aspect

A cell (optionally according to any other Configuration), comprising a nucleic acid, wherein the nucleic acid comprises a gene encoding a product of interest (P1), the gene comprising a nucleotide sequence (NS1) encoding P1 and a regulatory region 5′ of NS1 that comprises a promoter (Px) for controlling the expression of NS1, wherein the combination of Px and NS1 is heterologous to the cell and Px is regulatable by xylose.

In a third Aspect

A nucleic acid vector comprising a gene as recited in the first or second Aspect.

### BRIEF DESCRIPTION OF FIGURES

FIG. 1. plasmid map of p1364. The plasmid is annotated with relative gene locations and their functions. The plasmid contains the functions of plasmid replication (pBBR ori+rep) and RP4 origin of transfer (mob region encompassing oriT and relaxase).
araC—regulatory gene
pBAD—inducible promoter regulated by the presence of arabinose
cas3—gene encoding E. coli Type I-E Cas3 protein
casA-E—Genes encoding Cascade proteins
Prham—inducible promoter regulated by the presence of rhamnose
Bba B1003 Terminator—Artificial terminator
rmtB—16S rRNA methylase gene. Provides resistance to amikacin and gentamicin
sfgfp—gene encoding superfolder green fluorescent protein
kanR—kanamycin resistance gene
mob—mobilization gene. Requered for conjugal transfer of DNA
pBBRori—origin of replication
pBBR rep—gene encoding replication initiantion protein
CRISPR array—consists of E. coli direct repeats and one spacer

FIG. 2. Conjugative transfer of p1364. Transconjugants formed when p1364 is conjugated from the JKE201 strain encoding the RP4 transfer genes. A control lacking the oriT was included.

FIG. 3. GFP expression from p1364. The expressing level of GFP from cells harvested at an OD600 of 1. The b52 strain was included as an autofluorescence control.

FIG. 4. The self-targeting plasmid is lost rapidly upon CRISPR/cas induction. Proportion of GFP-positive colonies for induced and non-induced b52 containing the p1364 self-targeting plasmid over time.

FIG. 5. PCR screening for the p1364 plasmid backbone. Gel electrophoresis showing no presence of the p1364 in all picked GFP negative colonies from the 24 h timepoint.

FIG. 6. Chromosomal CRISPR/cas targeting allows for rapid removal of plasmid host. The change in host cell viable counts upon induction of CRISPR/cas system targeting the chromosome (IptA) of E. coli MG1655. Error-bars show standard deviation of 3 replicates.

FIG. 7. Various schematics illustrating pathways that may be use the invention.

FIG. 8: (i) Overview of GFP reporter plasmids with the xylitol-inducible regulatory system. (ii) Depicted is a genomic region from Morganella morganii strain ZJG812 (genome ID: CP064831.1) with structural genes highlighted related to xylitol uptake and metabolism: NAD(P)-dependent alcohol dehydrogenase (IZ184_04875 gene), xylulokinase (xylB), sugar ABC transporter ATP-binding protein (IZ184_04865 gene), ABC transporter permease (OZ184_04860 gene), substrate-binding domain-containing protein (IZ184_04855). These genes are expressed from a negatively inducible promoter (promoter) controlled by a LacI family DNA-binding transcriptional regulator (IZ184_04885 gene). A predicted D-lyxose/D-mannose family sugar isomerase (IZ184_04880 gene) is found in-between the promoter and the repressor. Gene annotations were made based on a sequence similarity search against the public database.

FIG. 9: Activity of xylitol-inducible promoter in response to xylitol. GFP reporter plasmids encoding the xylitol-inducible promoter system with a full-length transcriptional regulator (pSNP1902) or with a loss-of-function transcriptional regulator (pSNP103) were tested within a bSNP463 strain background in response to increasing amounts of xylitol in the growth medium (LB). Data shown is the fluorescence emission after 24 hours of incubation (normalized, see Example 2, section 3.2.2). Error bars indicate standard deviations based on three biological replicates.

FIG. 10: Activity of xylitol-inducible promoter in a minimal medium with increasing concentrations of xylitol. GFP reporter plasmids encoding the xylitol-inducible promoter system with a full-length transcriptional regulator (pSNP1902) was tested within a MG1655 (bSNP230) strain background growing in a minimal medium supplemented with glycerol and increasing amounts of xylitol. Data shown here is the fluorescence emission after 24 hours of incubation (normalized, see Example 2, section 3.2.2). Error bars indicate standard deviations based on three biological replicates.

FIG. 11: Growth of reporter plasmid carrying strain in minimal medium with and without glycerol and/or xylitol. The E. coli strain MG1655 (bSNP230) carrying the xylitol-inducible GFP reporter plasmid pSNP1902 was grown for 24 hours in minimal medium supplemented with glycerol (0.4%(v/v)) with or without xylitol (0.5% (w/v)). Error bars indicate standard deviations based on two biological replicates. The trends from these growth profiles (i.e. higher amounts of xylitol leads to slower growth) was also observed from the growth profiles of the remaining strains.

FIG. 12: Activity of the promoter in the presence of the xylitol ABC transporter system. E coli bSNP230 strains containing the GFP reporter plasmid (pSNP1902) either with or without a co-resident plasmid containing the xylitol ABC transporter (pSNP1939) were grown for 24-hours in minimal medium supplemented with 0.4% (v/v) glycerol (A) or 0.4% (w/v) glucose (B) in the presence or absence of xylitol. The cell-density adjusted fluorescence emission after 24 hours of incubation are shown. Error bars indicate standard deviations based on two (A) and three (B) biological replicates, respectively.

FIG. 13: Growth profiles of the ABC transporter-carrying strains on minimal medium in the absence or presence of xylitol. bSNP230 strains containing the GFP reporter plasmid (pSNP1902) either with or without a co-resident plasmid containing the xylitol ABC transporter (pSNP1939) were grown for 24-hours in minimal medium supplemented with 0.4% (v/v) glycerol (A) or 0.4% (w/v) glucose (B) in the presence or absence of xylitol. Error bars indicate standard deviations based on two (A) and three (B) biological replicates, respectively.

### DETAILED DESCRIPTION

The invention relates to methods and means for temporally regulating the production of products of interest (herein called P1, eg, proteins or RNAs) in microbiota of subjects. such as gut microbiota of humans or animals. For example, in this way microbiota can usefully be modified for expression of products in a controlled and possibly reversible manner. This can be useful to enable controlled engineering of microbiota of subjects in a way that enables, for example, production of desirable levels of P1 followed by controlled reduction in expression, eg, for reversion to a pre-engineered level of P1. To this end, the invention provides the following illustrative embodiments.

In a First Configuration, there is provided:

At least one nucleic acid vector for transfer into a host cell of a microbiota, the vector(s) comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to bind to vector nucleic acid to regulate expression of P1;
   wherein
(c) NS1 is under the control of a first promoter (eg, a constitutive or inducible promoter) for expression of P1; and
(d) NS2 is under the control of a second promoter that is regulatable for expression of P2, wherein binding of a regulator agent (R) to the vector nucleic acid regulates the second promoter, thereby regulating the expression of P2 and P1 (eg, R binds an operator that is operatively connected to the second promoter).

Said “at least one vector” may relate to one vector or a plurality of vectors, eg, first and second vectors, eg, 2 vectors. Herein, where features are described in the context of one vector or “the vector”, the skilled person will realise that the features may apply mutatis mutandis to said “at least one vector” such as a first and a second vector, or such as a plurality of vectors. The one vector may be further according to the vector of the Fourth Configuration. Each of said first and second, 2 or plurality of vectors may be further according to the vector of the Fourth Configuration. This is advantageous to limit the presence or spread of NS1 and/or NS2 (thus limiting P1 and/or P2 respectively) in the microbiota or subject comprising the microbiota.

For example, said at least one vector comprises a first vector and a second vector, wherein the first vectors comprises NS1 (and optionally not NS2) and the second vector comprises NS2 (and optionally not NS1) and the vectors are capable of co-existing in the host cell for expression of Pt and P2. Thus, the first vector may comprise NS1 and not NS2; and the second vector may comprise NS2 and not NS1. The first and second vectors are capable of being transferred into the same host cell and co-existing in the cell, whereby P1 and P2 can be expressed in the cell. For example, said at least one vector is one vector that comprises both NS1 and NS2.

A microbiota may be in any environment, eg, in soil or a waterway, comprised by a plant, or comprised by a human or animal subject. As the skilled person will know, a microbiota may comprise bacteria, archaea, fungi and viruses.

A First Aspect of the First Configuration provides:

A nucleic acid vector for transfer into a host cell of a microbiota, the vector comprising a nucleic acid that comprises
a) An expressible nucleotide sequence of interest (NS1) for producing a product of interest (P1) in the host cell; and
b) An expressible nucleotide sequence (NS2) for producing a regulator product (P2) in the host cell, wherein P2 is operable in the host cell to bind to the vector nucleic acid to regulate expression of P1;
Wherein
c) NS1 is under the control of a first promoter (eg, a constitutive or inducible promoter) for expression of P1; and
d) NS2 is under the control of a second promoter that is regulatable for expression of P2, wherein binding of a regulator agent (R) to the vector nucleic acid regulates the second promoter, thereby regulating the expression of P2 and P1.

As more fully described herein, some advantages of specific aspects of the invention may be
inducible self-targeting of the vector to remove nucleotide sequence(s) of interest previously spread in a microbiota to which the vector has been exposed. This would leave the mictobiota without a trace of the vector nucleic acid or with reduced prevalence of the vector nucleic acid, such as once a desired expression level of a NS1 product has been achieved;
killing the donor (carrier) bacteria by a CRISPR/cas (or other nuclease) vector-borne system that, upon induction, cuts the donor cell genome (eg, chromosome) and kills the donor cells—this being useful to clear the microbiota of donor cells (and thus reduce transmission of the vector in the microbiota) once a desired level of NS1 product expression has been achieved—and also useful as a safety off-switch system to reduce the vector nucleic acid in microbiota and other environments;
real-time regulation of production of NS1 product; feedback loop(s) can be employed to remove the vector nucleic acid as response to the produced compound or physiological signal(s);
localized on/off switches to ensure production of NS1 product at certain spatial sites, in an environment. eg, in a microbiota (eg, gut microbiota) of a human or animal subject.

Each host cell may be a bacterial, fungal (eg, yeast) or archaeal cell. Preferably, each host cell is a microbial cell. Preferably, each host cell is a bacterial cell. Preferably, each host cell is an archaeal cell.

Preferably, each host cell is a cell of a commensal or probiotic bacterial cell species found in human or animal microbiota. Preferably, each host cell is a cell of a commensal or probiotic bacterial cell species of a human or animal microbiota. Preferably, each host cell is a microbial (eg, bacterial) cell of a human or animal gut microbiota species.

The nucleic acid may be DNA or RNA. For example, the nucleic acid is DNA.

A host cell with reference to the First to Third Configurations is interchangeably referred to herein as a target cell. A host cell with reference to the Fourth Configurations is interchangeably referred to herein as a carrier or donor cell.

In an example, P1 is secreted from the host cell(s). In an example, P1 is expressed from NS1 in the host cell as a amino acid sequence comprising a signal peptide for secretion of P1 from the cell. In an example, P1 is not secreted from the host cell(s). P1 may be a protein or RNA (eg, a mRNA).

In an embodiment, P1 is not expressed in the microbiota prior to carrying out the method of the invention.

P2 may upregulate P1 expression. In an embodiment, P1 is expressed in the subject prior to carrying out the method of the invention, wherein carrying out the method causes at least a 50, 60, 70, 80, 90, 100, 200, 300, 400, 500,600, 700, 800, 900, 1000, 2000, 3000, 4000 or 5000% increase in the expression of Pt in the subject. The increase in expression may be determined by determining the relative levels of P1 in a tissue or fluid sample (eg, blood sample) that has been obtained from the patient. The increase in expression may be determined by determining the relative levels of P1 in a faecal sample that has been obtained from the patient.

P2 may upregulate P1 expression. In an embodiment, P1 is expressed in the microbiota (eg, gut microbiota of a human or animal subject) prior to carrying out the method of the invention, wherein carrying out the method causes at least a 50, 60, 70, 80, 90, 100, 200, 300, 400, 500, 600, 700, 800, 900, 1000, 2000, 3000, 4000 or 5000% increase in the expression of P1 in the microbiota. The increase in expression may be determined by determining the relative levels of P1 in a sample of the microbiota that has been previously obtained from the patient. The increase in expression may be determined by determining the relative levels of P1 in a tissue or fluid sample (eg, blood sample) that has been obtained from the patient. The increase in expression may be determined by determining the relative levels of P1 in a faecal sample that has been obtained from the patient (eg, wherein the microbiota is a gut microbiota).

P2 may downregulate P1 expression. In an embodiment, the method comprises administering R to the subject in step (c) of the method of the invention, whereby the expression of P1 in the subject is decreased by at least a 50, 60, 70, 80, 90, 100, 200, 300, 400, 500, 600, 700, 800, 900, 1000, 2000, 3000, 4000 or 5000% compared to the expression of P1 immediately before carrying out step (c). The decrease in expression may be determined by determining the relative level of P1 in a tissue or fluid sample (eg, blood sample) that has been obtained from the patient immediately before carrying out step (c) and comparing the level with the level of P1 in a similar sample (ie, tissue or fluid sample respectively) obtained after step (c) has been performed (eg, 0.5, 1, 2, 3, 4, 5, 6, 7, 8, 9, 10, 11, 12, 13, 14, 15, 16, 17, 18 19, 20, 21, 22, 23 or 24 hours, or 2, 3, 4, 5, 6, 7, 8, 9, 10, 11, 12, 13 or 14 days, or 3, 4, 5, 6, 7, or 8 weeks after the commencement of step (c)). The decrease in expression may alternatively be determined by determining the relative levels of P1 in faecal samples that have been obtained from the patient.

P2 may downregulate P1 expression. In an embodiment, the method comprises administering R to the subject in step (c) of the method of the invention, whereby the expression of P1 is decreased in the microbiota by at least a 50, 60, 70, 80, 90, 100, 200, 300, 400, 500, 600, 700, 800, 900, 1000, 2000, 3000, 4000 or 5000% compared to the expression of P1 immediately before carrying out step (c). The decrease in expression may be determined by determining the relative level of P1 in a tissue or fluid sample (eg, blood sample) that has been obtained from the patient immediately before carrying out 5 step (c) and comparing the level with the level of P1 in a similar sample (ie, tissue or fluid sample respectively) obtained after step (c) has been performed (eg, 0.5, 1, 2, 3, 4, 5, 6, 7, 8, 9, 10, 11, 12, 13, 14, 15, 16, 17, 18 19, 20, 21, 22, 23 or 24 hours, or 2, 3, 4, 5, 6, 7, 8, 9, 10, 11, 12, 13 or 14 days, or 3, 4, 5, 6, 7, or 8 weeks after the commencement of step (c)). The decrease in expression may alternatively be determined by determining the relative levels of P1 in faecal samples that have been obtained from the patient.

The second promoter may be an inducible promoter (wherein induction of promoter causes an increase in P2 expression) or a repressible promoter (wherein repression of promoter causes a reduction in P2 expression). Thus, binding of R to the nucleic acid may induce or repress the second promoter.

For example, a promoter herein is selected from Plac and Ptac (comprising lacO operator, lacI repressor), Ptet (comprising tetO operator, tetR repressor) and ParaBad (comprising araO operator, araC repressor). As will be familiar to the skilled person, it is possible to synthetically produce repressible promotors from native constitutive promoters by adding the tetO operator (or an analogue thereof) to the sequence and express tetR or analogues to repress them.

P2 may comprise a nucleic acid (eg, an RNA) or a protein (eg, a peptide). For example, P2 is a silencing RNA or protein that is capable of binding to the nucleic acid to inhibit expression of P1, eg, by binding to the first promoter a sequence that overlaps with the first promoter, or by binding to NS1.

P2 may be operable in the host cell to bind to the vector nucleic acid to downregulate expression of P1; and/or R may upregulate expression of P2. P2 may be operable in the host cell to bind to the vector nucleic acid to upregulate expression of P1.

P2 may be expressible in the host cell for the formation of a nuclease that is operable in the host cell to cut the nucleic acid. Preferably, the cut nucleic acid is degraded (optionally the vector comprising the nucleic acid is degraded), thereby downregulating the expression of P1 in the host cell. Preferably, the cut nucleic acid is degraded, thereby downregulating the expression of P1 in the microbiota. P2 itself can be the nuclease or a component thereof (wherein the component combines with one or more other components in the host cell to form the nuclease, such as an RNA-guided nuclease). Examples of suitable nucleases are an RNA-guided endonuclease or restriction endonuclease. For example the nuclease is a restriction nuclease selected from AatII, AbaSI, Acc65I, AccI, AciI, AciI, AcuI, AfeI, AfIII, AfIIII, AgeI, AhdI, AeI, AluI, AlwI, AlwNI, ApaI, ApaLI, ApoI, AscI, AseI, AsiSI, AvaI, AvaII, AvrII, BaeGI, BaeI, BamHI, BanI, BanII, BbsI, BbvCI, BbvI, BccI, BceAI, BcgI, BciVI, BclI, BfaI, BglI, BglII, BipI, BmgBI, BmrI, BmtI, BpmI, BpuEI, Bpul0I, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmFI, BsmI, BspCNI, BspEI, BspHI, Bsp1286I, BspMI, BsrBI, BsrDI, BsrFI, BsrGI, BsrI, BssHII, BssSI, BstAPI, BstBI, BstEII, BstNI, BstUI, BstXI, BstYI, BstZ171, Bsu36I, BtgI, BtgZI, BtsCI, BtsIMutI, BtsI, Cac8I, ClaI, CspCI, CviAII, CviKI-1, CviQI, DdeI, DpnI, DraI, DralII, DrdI, EaeI, EagI, EarI, EciI, Eco53kI, EcoNI, EcoOI09I, EcoP15I, EcoRI, EcoRV, Esp3I, FatI, FauI, Fnu4HI, FokI, FseI, FspEI, FspI, HaeII, HaeIII, HgaI, HhaI, HincII, HindIII, HinfI, HinP1I, HpaI, HphI, HpyAV, HpyCH4III, HpyCH4IV, HpyCH4V, Hpy99I, Hpy188I, Hpy 166II, Hpy188III, I-CeuI, I-SceI, KasI, KpnI, LpnPI, MboI, MboH, MfeI, MluCI, MluI, MlyI, MmeI, MnlI, MscI, MseI, MslI, MspAII, MspI, MspJI, MwoI, NaeI, NarI, Nb.BbvCI, Nb.BsmI, Nb.BsrDI, Nb.BssSI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaqCI, PciI, PflMI, PI-PspI, PI-SceI, PleI, PluTI, PmeI, PmlI, PpuMI, PshAI, PsiI, PspGI, PspOMI, PspXI, PstI, PvuI, PvuII, RsaI, RsrII, SacI, SacII, SalI, SapI, Sau96I, SbfI, ScaI, ScrFI, SexAI, SfaNI, SfcI, SfiI, SfoI, SgrAI, SmaI, SmlI, SnaBI, SpeI, SphI, SrfI, SspI, StuI, StyD4I, StyI, SwaI, TaqI, TfiI, TseI, Tsp45I, TspRI, Tth111I PflFI, XbaI, XcmI, XhoI, XhoI, PaeR71, XmaI, XmaI TspMI, XmnI and ZraI.

In an example, P2 comprises a guided nuclease that is programmable in the host cell to guide the nuclease to a target nucleotide sequence comprised by the nucleic acid, wherein the nuclease is capable of cutting the target sequence, whereby the nucleic acid sequence is degraded and P1 expression is inhibited from the nucleic acid.

In an example, the nuclease is operable to cut a target site comprised by the vector nucleic acid, wherein the target site is
(i) comprised by NS1;
(ii) not comprised by NS1;
(iii) comprised by NS2;
(iv) not comprised by NS2;
(v) comprised by a nucleotide sequence of the vector that encodes a CRISPR/cas system or a component thereof (eg, wherein the component is a nucleotide sequence that encodes a Cas, or wherein the component encodes a crRNA or guide RNA;
(vi) not comprised by a nucleotide sequence of the vector that encodes a CRISPR/cas system or a component thereof (eg, wherein the component is a nucleotide sequence that encodes a Cas, or wherein the component encodes a crRNA or guide RNA;
(vii) comprised by a nucleotide sequence that encodes a restriction endonuclease;
(viii) not comprised by a nucleotide sequence that encodes a restriction endonuclease;
(ix) comprised by the first promoter; or
(x) comprised by the second promoter.

A CRISPR/cas system comprises at least one Cas (eg, Cas3 (optionally also Cascade Cas, eg, CasA-E), Cas9, Cas12 or Cas13) and a cognate guide RNA that is capable of forming a Cas/guide RNA complex for recognising and binding to a protospacer sequence. In the above examples, the protospacer is comprised by the target site. The guide RNA may be a single guide RNA.

P2 may comprise
a) an RNA-guided nuclease;
b) an RNA that is operable for guiding an RNA-guided nuclease or a precursor of such an RNA; or
c) a restriction endonuclease.

Optionally, the precursor is an RNA, pre-cRNA or tracrRNA.

In an example, P2 is capable of binding to an operator (O) that is comprised by the nucleic acid and operatively connected to the first promoter (eg O is 5′ of the first promoter, eg, withing 200 or 100 kb 5′ of the promoter), wherein when P2 is bound to O the expression of P1 is reduced (eg, eliminated). In an example, P2 comprises a dead Cas nuclease (eg, a dCas9 or dCas3 or dCas12 or dCas13) wherein the dCas is capable of forming a dCas/guide RNA complex in the host cell that recognises and binds to a protospacer comprised by the nucleic acid to interrupt expression of P1 (eg, by interrupting promoter function of the first promoter).

Optionally, the guided nuclease is a Cas nuclease, TALEN, meganuclease or zinc finger nuclease, preferably a Cas nuclease. The nuclease may cut DNA or RNA, preferably DNA.

The nuclease may be operable to cut the nucleic acid at a predetermined sequence motif (a target site), optionally a protospacer sequence or restriction site. The protospacer may be a CRISPR/Cas protospacer. The restriction site may be cut by a restriction endonuclease or any other restriction nuclease disclosed herein. The nucleic acid may comprise a plurality (eg, at least 2 or 3, eg, 2, 3, 4, 5, 6, 7, 8, or 9) of said motifs. This may be useful for efficiency of cutting and destruction of the nucleic acid (or vector comprising the nucleic acid).

P1 may be an amino acid, protein (eg, peptide or a polypeptide) or RNA (eg, mRNA or silencing RNA) for human or animal therapy. For example P1 is a cytokine, growth factor, enzyme, hormone or antibody (or antibody chain or antibody fragment). For example, P1 is an antibody chain or antibody fragment, eg, a single domain antibody (AKA a dAb) or a nanobody. For example, the chain or fragment is a human antibody chain or fragment. For example, P1 is an antibody heavy chain that forms an antibody with an antibody light chain that is also expressed in the host cell. Preferably. the antibody, chain or fragment is capable of being secreted from the host cell. Preferably, the antibody, chain or fragment is secreted from the host cell. For example, P1 is an incretin, eg, an incretin peptide or a multimer thereof. Optionally, the incretin in selected from GLP-1, GIP, exendin-4 and insulin. For example, P1 comprises an antigen binding site of an antibody or a variable domain (eg, VH and/or VL domain) of an antibody, eg, wherein the antibody is selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin®; Mylotarg™; Gemtuzumab; Campath®: Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; pembrolizumab, nivolumab, atezolizumab, Kadcyla™; Ado-trastuzumab; Keytruda™, Opdivo™, Gazyva™ and Obinutuzumab. For example, P is selected from an insulin peptide, incretin peptide or peptide hormone. For example, the antibody is adalimumab. For example, the antibody is pembrolizumab. For example, the antibody is nivolumab. For example, the antibody is atezolizumab. For example, the antibody is dupilumab. For example, the antibody is tocilizumab. For example, the antibody is sarilumab. For example, the antibody is alirocumab. For example, the antibody is evolocumab. In an alternative the antibody is an anti-CD38 antibody, an anti-TNFa antibody, an anti-TNFR antibody, an anti-IL-4Ra antibody, an anti-IL-6R antibody, an anti-IL-6 antibody, an anti-VEGF antibody, an anti-EGFR antibody, an anti-PD-1 antibody, an anti-PD-L1 antibody, an anti-CTLA4 antibody, an anti-PCSK9 antibody, an anti-CD3 antibody, an anti-CD20 antibody, an anti-CD138 antibody, an anti-IL-1 antibody. In an alternative the antibody is selected from the antibodies disclosed in WO2007024715 at page 40, line 23 to page 43, line 23, the disclosure of which is incorporated herein by reference.

An antigen herein may be an antigen selected from the group consisting of ABCF1; ACVR1; ACVR1B; ACVR2; ACVR2B; ACVRL1; ADORA2A; Aggrecan; AGR2; AICDA; AWI; AIGI; AKAP1; AKAP2; AIYIH; AMHR2; ANGPT1; ANGPT2; ANGFTL3; ANGPTL4; ANPEP: APC; APOC1; AR; AZGPI (zinc-a-glycoprotein); B7.1; B7.2; BAD; BAFF; BAG1; BAI1; BCL2; BCL6; BDNF; BLNK; BLR1 (MDR 15); BlyS; BM P1; BMP2; BMP3B (GDFIO); BMP4; BMP6; BM P8; BMPRIA; BMPRIB: BM PR2; BPAG1 (plectin); BRCA1; CI9orflO (IL27w); C3; C4A; C5; C5R1; CANT1; CASPi; CASP4; CAVi; CCBP2 (D6/JAB61); CCL1 (1-309); CCL11 (eotaxin); CCL13 (MCP-4); CCL15 (MIP-id); CCL16 (HCC-4); CCL17 (TARC); CCL18 (PARC); CCL19 (M IP-3b); CCL2 (MCP-1); MCAF; CCL20 (MIP-3a); CCL21 (MIP-2); SLC; exodus-2; CCL22 (MDC/STC-1); CCL23 (M PIF-1); CCL24 (MPIF-2 I eotaxin-2); CCL25 (TECK): CCL26 (eotaxin-3): CCL27 (CTACK/ILC); CCL28; CCL3 (MIP-la); CCL4 (M IP-lb); CCL5 (RANTES); CCL7 (MCP-3); CCL8 (mcp-2); CCNA 1; CCNA2; CCND1; CCNEl; CCNE2; CCR I (CKR I/HM145); CCR2 (mcp-1RB/RA); CCR3 (CKR3/CMKBR3); CCR4; CCR5 (CM KBR5/ChemR13); CCR6 (CMKBR6/CKR-L3/STRL22/DRY6); CCR7 (CKR7/EBIl); CCR8 (CM KBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN I A (p2fWapl/Cipl); CDKN I B (p27Kipl); CDKNIC; CDKN2A (pl61NK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR 1; CMKOR1 (RDCI); CNR1; COL18AI; COL1AI; COL4A3; COL6A1; CR2; CRP; CSF1 (M-CSF); CSF2 (GM-CSF); CSF3 (GCSF); CTLA4; CTNNBI (b-catenin); CTSB (cathepsin B); CX3CL1 (SCYDi); CX3CR1 (V28); CXCL1 (GROI); CXCLIO (IP-10); CXCL1I (1-TAC/IP-9); CXCL12 (SDF1); CXCL13; CXCL14; CXCL16; CXCL2 (GR02); CXCL3 (GR03); CXCL5 (ENA-78 I LIX); CXCL6 (GCP-2); CXCL9 (MIG); CXCR3 (GPR9/CKR-L2); CXCR4; CXCR6 (TYMSTR ISTRL33 I Bonzo); CYB5; CYC1; CYSLTR1; DAB2IP; DES; DKFZp451JO118; DNCL1; DPP4; E2F1; ECGF1; EDGi; EFNAI; EFNA3: EFNB2; EGF; EGFR: ELAC2; ENG; EN01; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGFI (aFGF); FGF10; FGF11; FGF12; FGFI2B; FGF13; FGF14; FGF16; FGF17; FGF18; FGF19; FGF2 (bFGF); FGF20; FGF21; FGF22; FGP23; FGF3 (int-2): FGF4 (HST); FGF5; FGF6 (HST-2); FGF7 (KGF); FGF8; FGF9; FGFR3; FIGF (VEGFD); FILI (EPSILON); FILl (ZETA); FUl2584; FU25530: FLRTI (fibronectin): FLTI: FOS; FOSLI (FRA-1); FY (DARC); GABRP (GABAa); GAGEBI; GAGECI; GALNAC4S-65T; GATA3; GDF5; GFI1; GGT1; GM-CSF; GNAS1; GNRH1; GPR2 (CCRIO); GPR31; GPR44; GPR81 (FKSG80); GRCCIO (CIO); GRP; GSN (Gelsolin); GSTPI; HAVCR2; HDAC4; EDAC5; HDAC7A; HDAC9; HGF; HIFlA; HIPi; histamine and histamine receptors; HLA-A; HLA-DRA; HM74; HMOX1; HUMCYT2A; ICEBERG; ICOSL; 1D2; IFN-a; IFNA1; IFNA2; IFNA4; IFNA5; IFNA6; IFNA7; IFNB1; IFNgamma; TFNW1; IGBP1; TGF1; IGF1R; TGF2; IGFBP2; IGFBP3; TCFBP6; IL-1; IL10; IL10RA; IL10RB; rL11; IL11RA; IL-12; IL12A; IL12B; IL12RB1; IL12RB2; IL13; IL13RA1; IL13RA2; IL14; IL15; IL15RA; 116; IL17; TL17B; TL17C; IL 17R; IL18; TL18BP; IL18R1; IL18RAP; IL19; ILIA; TL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2 IL1RN; IL2; IL20; IL20RA; IL21R; IL22; IL22R; IL22RA2; IL23; IL24; IL25; I L26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; TL7R; IL8; IL8RA; IL8RB; IL8RB; L9; IL9R; ILK; INHA; INHBA; TNSL3; INSL4; TRAKI; IRAK2; ITGA; ITGA2; 1TGA3; ITGA6 (a6 integrin); ITGAV; ITGB3; ITGB4 (b 4 integrin); JAGi; JAKi; JAK3; JUN; K6HF; KAIl; KDR; MTLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; M IB1; midkine; M rF; M IP-2; MK167 (Ki-67); MMP2; M MP9; MS4A1; MSMB; MT3 (metallothionectin-ifi); MTSS 1; M UC 1 (mucin); MYC; MYD88; NCK2; neurocan; NFKB 1; NFKB2; NGFB (NGF); NGFR; NgR-Lingo; NgR-Nogo66 (Nogo); NgR-p75; NgR-Troy; NM El (NM23A); NOX5; NPPB; NROB1; NROB2; NR1D1; NR 1D2; NR 1H2; NR1H3; NR1H4; NR 112; NR 1I3; NR2C1; NR2C2; NR2EI; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRDI; P2RX7; PAP; PARTI; PATE; PAWR; PCA3; PCNA; PDGFA; PDGFB; PECAM1; PF4 (CXCL4); PGF; PGR; phosphacan; PIAS2; P1K3CG; PLAU (uPA); PLG; PLXDCI; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p2TRac2); RARB; RGSI; RGS13; RGS3; RNF110 (ZNF144); ROB02; S100A2; SCGBID2 (lipophilin B); SCGB2A1 (mammaglobin 2); SCGB2A2 (mammaglobin 1); SCYE1 (endothelial Monocyte-activating cytokine); SDF2; SERPINAI; SERPTNIA3; SERPTNB5 (maspin); SERPINEI (PAT-i); SERPTNFI; SHBG; SLA2; SLC2A2; SLC33AI; SLC43A1; SLIT2; SPPI; SPRRIB (Spri); ST6GAL1; STABI; STAT6; STEAP; STEAP2; TB4R2; TBX21; TCPIO; TDGF1; TEK; TGFA; TGFB1; TGFBII; TGFB2; TGFB3; TGFBI; TGFBR1; TGFBR2; TGFBR3; THIL; THBSI(thrombospondin-1); THBS2; THBS4; THPO; TIE (Tie-i); T]MP3; tissue factor; TLRIO; TLR2; TLR3; TLR4; TLR5; TLR6; TLR7; TLR8; TLR9; TNF; TNF-α; TNFAIP2 (B94); TNFAIP3; TNFRSFI IA; TNFRSFIA; TNFRSFIB; TNFRSF21; TNFRSF5; TNFRSF6 (Fas); TNFRSF7; TNFRSF8; TNFRSF9; TNFSFIO (TRAIL); TNFSFI I (TRANCE); TNFSF12 (APO3L); TNFSF13 (April); TNFSF13B; TNFSF14 (HVEM-L); TNFSFI 5 (VEGI); TNFSFI 8; TNFSF4 (0X40 ligand); TNFSF5 (CD40 ligand); TNFSF6 (FasL); ThFSF7 (CD27 ligand); TNFSF8 (CD30 ligand); TNFSF9 (4-1BB ligand); TOLLIP; Toll-like receptors; TOP2A (topoisomerase lia); TP53; TPM 1; TPM2; TRADD; TRAFi; TRAF2; TRAF3; TRAF4; TRAF5; TRAF6; TREM 1; TREM2; TRPC6; TSLP; TWEAK; VEGF; VEGFB: VEGFC; versican; VHL C5; VLA-4; XCLI (lymphotactin); XCL2 (SCM-1b); XCR1 (GPR5/CCXCR1); YYI; and ZFPM2.

For example, P1 comprises a VEGF binding site of aflibercept, eg, anti-VEGF FLT1 and/or KDR domain(s).

For example, P1 comprises (or is) an incretin, an insulin peptide, a GLP-1 (glucagon-like peptide-1 (GLP-1) peptide, a GIP (glucose-dependent insulinotropic polypeptide) peptide, an exendin (eg, exendin-4) peptide, a peptide hormone, a prolactin or prolactin peptide, a ACTH or ACTH peptide, a growth hormone or growth hormone peptide, a vasopressin or vasopressin peptide, an oxytocin or oxytocin peptide, a glucagon or glucagon peptide, a insulin or insulin peptide, a somatostatin or somatostatin peptide, a cholecystokinin or cholecystokinin peptide, a gastrin or gastrin peptide, a leptin or leptin peptide, an antibody binding site (eg, a scFv or Fab) or variable domain thereof, a TCR binding site (eg, a scTCR) or domain thereof, a TCR Vα/Vβ binding site or variable domain thereof, a TCR Vγ/Vδ binding site or variable domain thereof, an antibody single variable domain binding site, or an FcAb binding site. In an example, P1 comprises at least one copy of a GLP-1 and at least one copy of another incretin (eg, an Exendin-4). For example, there is one copy of GLP-1 and one copy of the other increting. Preferably, in the examples in this paragraph, P1 is a secreted or host cell surface-exposed protein. Optionally. any GLP-1 herein is GLP-l(7-37)-Pro9. Optionally, any incretin herein is an Exendn-4 or Peptide Y. Optionally, any P1 or incretin herein is DURAGLUTIDE™.

P1 may comprise an antigen binding site. A binding site herein may, for example, be an antigen (eg, cytokine or growth factor, eg, VEGF or EGFR) binding site of a receptor (eg, KDR or FIt). A binding site herein may, for example, be a binding site of Eyelea™, Avastin™ or Lucentis™, eg, for ocular or oncological medical use in a human or animal. When the antigen is VEGF, the vector or method may be for treatment or prevention of a cancer or ocular condition (eg, wet or dry AMD or diabetic retinopathy) or as an inhibitor of neovascularisation in a human or animal subject.

For example, P1 is a metabolism pathway component, eg, an enzyme or reagent in the pathway. For example, P1 is an intracellular enzyme in the target cell (ie, host cell). For example, P1 is a secreted enzyme (eg, secreted from the target cell). The pathway herein may be in the target cell or it may be outside the target cell. For example, the pathway is a pathway inside a different cell comprised by the microbiota of which the target cell is a component. The different cell may be a carrier cell or it may be an endogenous cell of the microbiota (ie, any cell of the microbiota except the target cell or a carrier cell). The pathway may comprise one or more as a product or intermediate:—
a short-chain fatty acid (SCFA)
a lipid
an indole derivative or serotonin (eg, wherein the pathway is for the conversion of tryptophan (Trp) to one or more indole derivatives or serotonin)
a hormones or incretin (eg, GLP-1, oxytocin)
one or more antigens (eg, to stimulate immune response for vaccination of the subject).

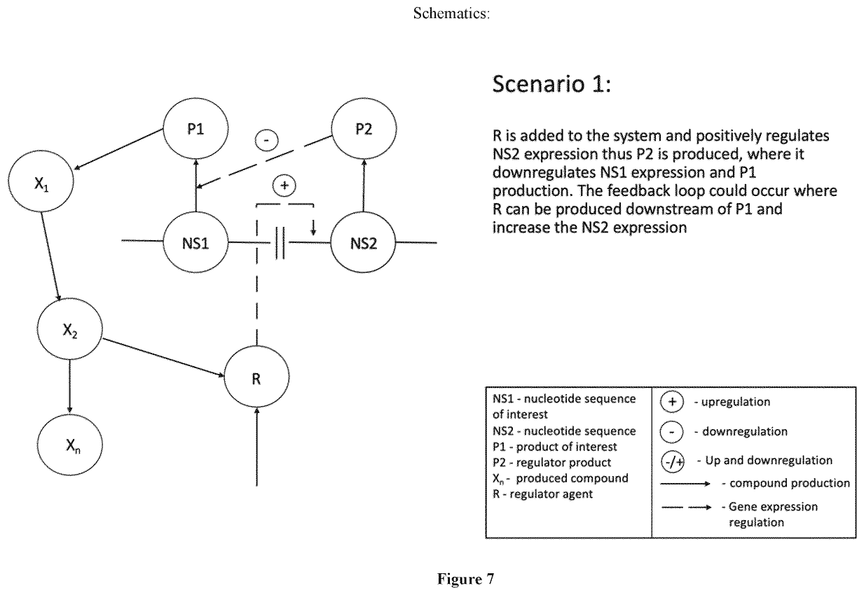

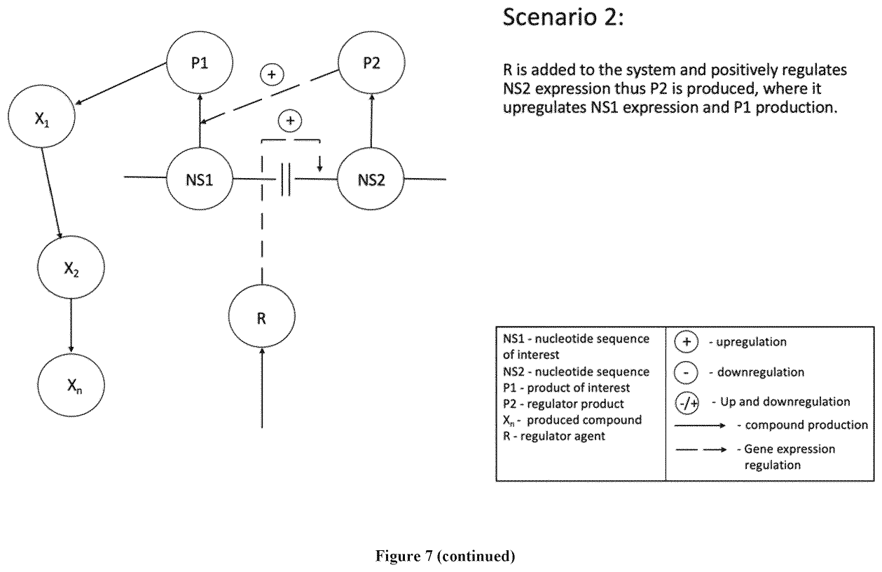

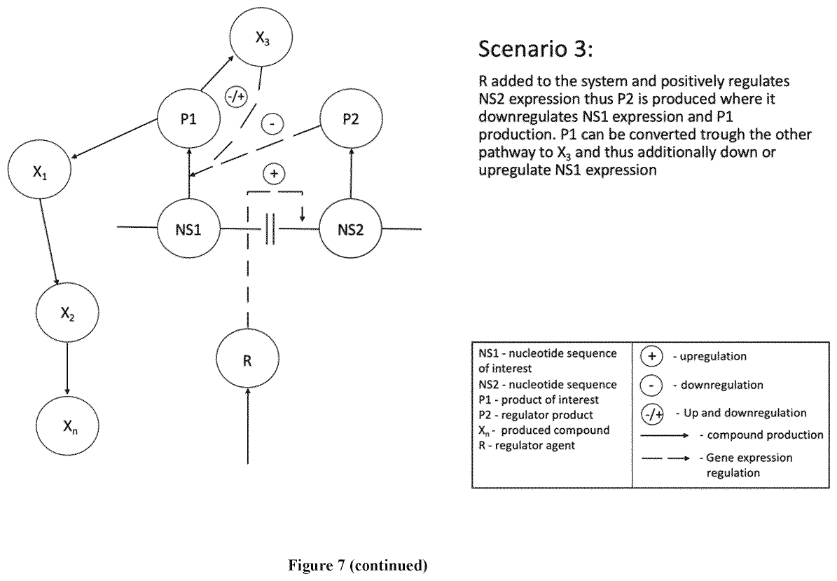

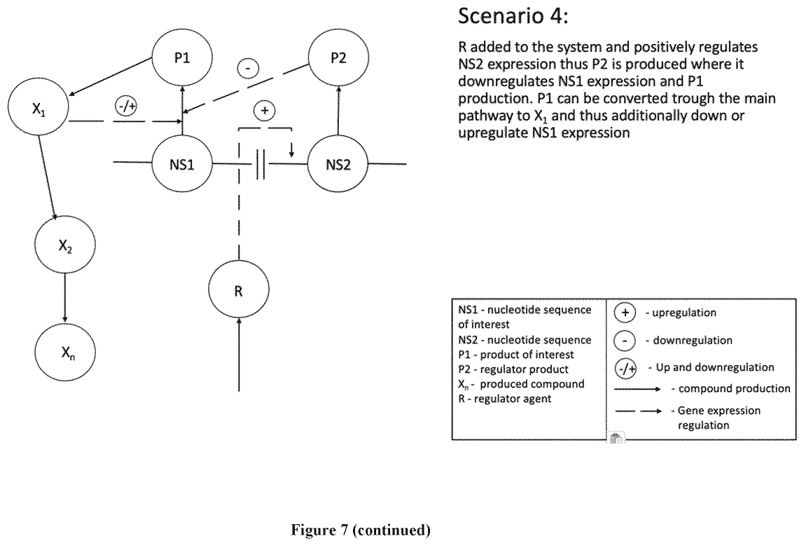

Optionally, R upregulates P2 expression, P2 downregulates P1 expression and optionally P1 is a component in a metabolic pathway (eg, in the microbiota or target cell or in a subject or environment comprising the microbiota or target cell) wherein a production product (X) of the pathway downstream from P1 causes regulation of P1 or P2 expression. See, for example, Scenarios 1, 3 and 4 (FIG. 7). In an embodiment, X is R or a precursor of R. In an embodiment, X regulates the first and/or second promoter. In an embodiment, X upregulates P1 or P2 expression. In an embodiment, X upregulates P1 or P2 expression.

Optionally, R upregulates P2 expression, P2 upregulates P1 expression and optionally P1 is a component in a metabolic pathway (eg, in the microbiota or target cell or in a subject or environment comprising the microbiota or target cell) wherein a production product (X) of the pathway downstream from P1 causes regulation of P1 or P2 expression. See, for example, Scenario 2 (FIG. 7). In an embodiment, X is R or a precursor of R. In an embodiment, X regulates the first and/or second promoter. In an embodiment, X upregulates P1 or P2 expression. In an embodiment, X upregulates P1 or P2 expression.

In an example, P1 is a secreted or cell-surface exposed protein antigen. This is useful, for example, to vaccinate the subject, eg, where the antigen is an antigen of a pathogen (such as a bacterium or virus, eg, a coat protein, such as spike protein, eg, a SARS-Cov or SARS-Cov-2 or influenza antigen).

In an example, P1 is a secreted antagonist of a target ligand in the subject. For example, binding of P1 to the ligand may inhibit or neutralise the ligand or mark it for destruction in the subject (eg, by immune cells of the subject). For example, the antagonist may comprise an antibody fragment (such as a nanobody or any other antibody single variable domain) comprising a binding site for the target ligand. The ligand may be, for example,
A curli protein (eg, an E. coli curli protein);
TMA (trimethylamine);
A gluten;
A bile acid;
Cholesterol or PCSK9: or
A bacterial toxin (eg, a toxin encoded by pks (eg. E. coli pks), C. difficile toxin, V. cholerae toxin, anthrax toxin or B. fragilis toxin).

In an embodiment, P1 is toxic to cells of the same species as the host cell. This may be useful for killing cells of such species in the microbiota or for reducing the growth or proliferation of such cells, such as when the cells are detrimental to the health of the subject.

In an embodiment, P1 is a transcription or translation regulator in cells of the same species as the host cell. Thus, up- or down-regulation of P1 expression may advantageously act on one or a plurality of genes in the genome of the host cell.

Optionally, R is an amino acid, protein, carbohydrate (eg, a sugar), lipid, metal ion or nucleic acid (eg, RNA). R may be a sugar alcohol. eg, xylitol, glycerol, arabitol, erythritol, isomalt, HSHs, lactitol, maltitol, mannitol or sorbitol, preferably xylitol. R may be an antibiotic. R may be a metabolite of a metabolic pathway that operates in the subject or in the microbiota. R is optionally a metabolite of P1 or a metabolite produced in a pathway comprising P1. For example, P1 can be a secreted enzyme (ie, secreted from the host cell) that is capable of acting in a pathway in the subject that produces R. For example, P1 can be metabolised in a pathway that produces R (eg, P1 is metabolised to directly produce R, or to indirectly produce R). The pathway may be inside the target host cell or may be outside the target host cell (eg, in a neighbouring cell in the microbiota of which the target cell is a component). The effect of R on P1 expression may be dose dependent (or it may not). The skilled person will readily be able to determine (eg, by way of titrating doses in assays) the appropriate amount of R to use to provide a desired effect on the microbiota.

As exemplified herein, R may be xylitol. Operons of genes related to the conversion/utilization of sugars can be induced by the sugars themselves by virtue of their binding to the relevant transcriptional repressor proteins. For the present study it was decided to investigate a negatively repressible promoter predicted to be induced by xylitol. This 5-carbon sugar alcohol has several advantages. First of all, xylitol is generally regarded as safe for human consumption by the FDA (Xiang et al., 2021). Furthermore, over half of ingested xylitol is not adsorbed by human cells instead reaching the gastrointestinal tract where it is taken up by the microbiome (Livesey, 2003). Finally, xylitol serves as a metabolite for some bacteria: It is either taken up directly through an ABC-type transporter complex (Madigan et al., 2015) and/or generated through reduction of the corresponding sugar D-xylose. Followingly, xylitol is typically dehydrogenated and phosphorylated to xylulose-5-phosphate which is further catabolized in the pentose phosphate pathway.

For example, R is an amino acid. For example, R is selected from Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine and Valine. In an embodiment, R is Tryptophan. For example, R is a protein (eg, a peptide), eg, R is selected from a nisin, sakacin A and sakacin P, For example, R is a bacteriocin. For example, R is a carbohydrate, eg, R is a sugar, such as selected from 1-arabinose, 1-rhamnose, xylose and sucrose. For example R is a metal ion, eg, R is selected from Fe²⁺, Mn²⁺, Co²⁺, Hg²⁺ and Cu²⁺. For example, R is a lipid, eg, proprionate. For example, R is a fatty acid. For example, R is a nucleic acid, eg, R is an RNA. For example, R is a benzene compound, eg, a substituted benzene compound or benzoic acid.

Optionally, the vector is an ICE (integrative and conjugative element), plasmid (eg, a conjugative plasmid), transduction particle (eg, a phage or non-self-replicative transduction particle) or nanoparticle. In an embodiment, the plasmid comprises an oriT and oriV. The plasmid may be self-conjugating. The plasmid may be a shuttle plasmid, ie, a plasmid that can propagate in at least two different host species. The plasmid may comprise a transposon or an ICE (or mobilizable part thereof) that comprises the nucleic acid comprising NS1 and NS2. For example, the transposon or ICE may be a Barteroides transposon or ICE. The plasmid may comprise an oriT and be mobilisable in the presence of a conjugative system, eg, a system found on other plasmids or integrative and conjugative element (ICE) in the host cell or microbiota. In an example, a such conjugative system is comprised by the genome of the donor cell or carrier cell. In an example, additionally or alternatively, a conjugative system is comprised by the genome of a recipient cell. The conjugative system may be carried on a chromosome or episome of a carrier cell as described herein (ie, system in trans to the plasmid). The conjugative system may be comprised by the plasmid itself (ie, system in cis). The system may be comprised by cells in the microbiota that neighbour the host cell into which the plasmid has been transferred; in this way the plasmid can be spread between neigbouring cells, thereby propagating the plasmid vector in the microbiota. This can be useful to amplify the P1 expression in the microbiota. Similarly, with a self-conjugating plasmid (ie, the conjugative system is provided along with oriT on the plasmid), the plasmid can spread in the microbiota.

The vector may be a conjugative plasmid comprised by a carrier cell (eg, a bacterial carrier cell). For example, the carrier cell and vector is for administration to a microbiota of a human or animal subject. For example, the carrier cell is a cell of commensal or probiotic bacterial cell species of a human or animal microbiota. Additionally or alternatively, the carrier cell is a cell of a human or animal gut microbiota species.

The conjugation genes of conjugative plasmids such as those of the Inc groups: P, N, W, or X show similarity at the protein level to the VirB system of Agrobacterium, itself constituting a prototypic Type IV secretion system (T4SS). These are often smaller and probably the most minimal systems around. The genes necessary for conjugation of the prototypical VirB plasmid pTI and the protein homologs in the incN (somewhat related) and incF (distantly related) plasmid families are shown in Table 4.

Optionally, the conjugation system is a VirB, IncN or IncF conjugation system, or a homologue or orthologue thereof.

Each vector may be a self-conjugative plasmid comprising an oriT and a conjugation system for transferring the plasmid between cells in the microbiota, wherein the conjugation system is a VirB, IncN or IncF conjugation system, or a homologue or orthologue thereof. The carrier (or donor) cell and said plasmid vector(s) may between them comprise a conjugation system for transferring the plasmid between cells in the microbiota, wherein the conjugation system is a VirB, IncN or IncF conjugation system, or a homologue or orthologue thereof, wherein each vector comprises an oriT. The host (or recipient) cell and said plasmid vector(s) may between them comprise a conjugation system for transferring the plasmid between cells in the microbiota, wherein the conjugation system is a VirB, IncN or IncF conjugation system, or a homologue or orthologue thereof, wherein each vector comprises an oriT.

For example, the system is the system of VirB pasmid pTIm, an IncN plasmid or an IncF plasmid, or the system is a homologue or orthologue thereof.

For example, the system comprises (i) VirB genes virB1-11 and virD4, or homologues or orthologues of said genes; (ii) IncN genes traA-G, traJ, traL, traM, traN and traO, or homologues or orthologues 30 of said genes; (iii) Inc F genes traA-E, traG, traH, traK, traL and ORF196, or homologues or orthologues of said genes; or (iv) IncF genes traA-T, traK, traL, traM, traW and traU (and optionally at least one or all of traX, traN, finO, trbI and trbB), or homologues or orthologues of said genes. For option (iv), reference is made to Front Mol Biosci., 2016 Nov. 10; 3:71. doi: 10.3389/fmolb.2016.00071. eCollection 2016, “Comparative Genomics of the Conjugation Region of F-like Plasmids: Five Shades of F”, Raul Fernandez-Lopez et al.

The invention also provides, according to the Fourth Configuration:

A nucleic acid vector for transfer into a host cell of a microbiota, wherein the vector is comprised by a carrier cell (eg, a bacterial cell) and encodes
c) a nuclease (optionally an RNA-guided nuclease or restriction endonuclease) that is operable in the carrier cell to cut a chromosome or episome (which is not the vector of the invention) of the carrier cell, optionally wherein the chromosome or episome is degraded; and/or
d) an RNA that is operable in the carrier cell for guiding an RNA-guided nuclease or a precursor of such an RNA, wherein the RNA guides the nuclease to cut a chromosome or episome (which is not the vector) of the carrier cell, optionally wherein the chromosome or episome is degraded;
   wherein the vector comprises one or more regulatable promoters for regulating expression of the nuclease of (a) and/or the RNA or component of (b) in the carrier cell.

The vector may have any of the vector features disclosed herein. The episome may be a plasmid.

In an embodiment, the vector comprises an inducible or repressible promoter that regulates expression of the nuclease of (a) and/or the vector comprises an inducible or repressible promoter that regulates expression of the RNA or component of (b), preferably wherein the promoter(s) are inducible promoters. In an alternative, the promoter(s) are repressible promoters. In an example, components (a) and (b) are under the control of different promoters. In an example, components (a) and (b) are under the control a common promoter

In an embodiment, the vector comprises an inducible promoter that regulates expression of the nuclease of (a). In an embodiment, the vector comprises an inducible promoter that regulates expression of the RNA or component of (b). Alternatively, the vector comprises an repressible promoter that regulates expression of the RNA or component of (b).

In an embodiment, the vector comprises an repressible promoter that regulates expression of the nuclease of (a). In an embodiment, the vector comprises an inducible promoter that regulates expression of the RNA or component of (b). Alternatively, the vector comprises an repressible promoter that regulates expression of the RNA or component of (b).

Optionally, the guided nuclease is a Cas nuclease, TALEN, meganuclease or zinc finger nuclease, preferably a Cas nuclease. For example, the guided nuclease is a Cas3. For example, the guided nuclease is a Cas9. For example, the guided nuclease is a Cas12 (eg, Cas12a). For example, the guided nuclease is a Cas13 (eg, Cas13a).

The nuclease in the Fourth Configuration is the same guided nuclease as in the First Configuration.

The nuclease in the Fourth Configuration is operable to cut the chromosome or episome in the carrier cell at a predetermined sequence motif, optionally a protospacer sequence or restriction site. Preferably, cutting of the carrier cell chromosome or episome kills the carrier cell or reduces growth or proliferation of the carrier cell, most preferably wherein the cell is killed. This is useful to reduce the transmission of the vector nucleic acid, such as wherein a microbiota or environment has been exposed to the vector. This provides a useful way of regulating expression of products from the vector, such as expression in recipient cells into which the vector nucleic acid has been transferred.

In an example, the vector comprises an oriT for transfer into the host cell, optionally wherein the vector is a conjugative plasmid.

In a preferred embodiment
a) the vector is a conjugative plasmid;
b) the vector comprises an inducible promoter that regulates expression of the nuclease of (a) and/or the vector comprises an inducible promoter that regulates expression of the RNA or component;
c) optionally the guided nuclease is a Cas nuclease; and
   cutting of the carrier cell chromosome or episome kills the carrier cell or reduces growth or proliferation of the carrier cell.

According to the Fourth Configuration, there is provided:

A nucleic acid vector for transfer into a host cell of a microbiota, wherein the vector is comprised by a carrier cell (eg, a bacterial cell) and encodes
a) a nuclease (optionally an RNA-guided nuclease or restriction endonuclease) that is operable in the carrier cell to cut a chromosome or episome (which is not the vector) of the carrier cell, optionally wherein the chromosome or episome is degraded; and/or
b) an RNA that is operable in the carrier cell for guiding an RNA-guided nuclease or a precursor of such an RNA, wherein the RNA guides the nuclease to cut a chromosome or episome (which is not the vector) of the carrier cell, optionally wherein the chromosome or episome is degraded;
   wherein the vector comprises one or more regulatable promoters for regulating expression of the nuclease of (a) and/or the RNA or component of (b) in the carrier cell;
   wherein cutting of the carrier cell chromosome or episome kills the carrier cell or reduces growth or proliferation of the carrier cell, preferably wherein the cell is killed.

There is also provides a plurality of carrier cells comprising the vector. The cutting may reduce the number of carrier cells of said plurality at least 10⁵, 10⁶ or 10⁷-fold, eg, between 10⁵ and 10⁷-fold, or between 10⁵ and 10⁸-fold or between 10⁵ and 10⁹-fold. The skilled person will be familiar with determining fold-killing or reduction in cells, eg, using a cell sample that is representative of a microbiota or cell population. For example, the extent of killing or reduction is determined using a cell sample, eg, a sample obtained from a subject to which the carrier cells of the invention have been administered, or an environmental sample (eg, aqueous, water or soil sample) obtained from an environment (eg, a water source, waterway or field) that has been contacted with the carrier cells of the invention. For example, the cutting reduces the number of carrier cells of said plurality at least 10⁵, 10⁶ or 10⁷-fold and optionally the plurality comprises at least 100,000; 1,000,000; or 10,000.000 carrier cells respectively. Optionally, the plurality of carrier cells is comprised by a cell population of the microbiota, wherein at least 5, 6 or 7 log 10 of cells of the population are killed by the cutting, and optionally the plurality comprises at least 100,000; 1,000,000; or 10,000,000 carrier cells respectively. Optionally, the cutting kills at least 99%, 99.9%, 99.99%, 99.999%, 99.9999% or 99.99999% cells of said plurality of carrier cells.

There is provided a method of killing a plurality of carrier cells comprised by a microbiota, the carrier cells comprising vectors of the Fourth Configurations, wherein the method comprises upregulating expression of the nuclease of (a) and/or the RNA or component of (b) in the carrier cells (eg, by inducing the regulatable promoter(s)) whereby the genomes of the carrier cells are cut and the cells are killed. Preferably, chromosomes of the cells are cut. The method optionally kills at least 99%, 99.9%, 99.99%, 99.999%, 99.9999% or 99.99999% cells of said plurality of carrier cells. In an example, the method kills all (or essentially all) of the cells of said plurality of carrier cells. In an example, the method is kills 100% (or about 100%) of the plurality of carrier cells. Preferably all carrier cells comprised by the microbiota are cells of said plurality.

Preferably, at least 90, 91, 92, 93, 94, 95, 96, 97, 98 or 99% of the carrier cells are killed.

A conjugative plasmid herein may be a self-conjugative plasmid (ie, wherein the plasmid comprises an oriT and encodes all proteins required to mobilise the plasmid for conjugative transfer between cells).

For any Configuration herein, the host cell (eg, donor, carrier or recipient cell) is a cell of a species found in a microbiota (eg, gut microbiota) of humans or animals. In a preferred example, the species is a Bacteroides species. In a preferred example, the species is E col. The host cell may be a cell of commensal or probiotic bacterial cell species of a human or animal microbiota. For example, the species is selected from any species in Table 1, preferably a Bacteroides species (eg, Bacteroides thetaiomaomicron, Bacteroides vulgatus, Bacteroides uniformis or Bacteroides ovatus) or Clostridales species, (eg, Clostridioides difficile or Clostridium disporicum).

In an embodiment,
a) Pt is a protein or RNA (eg, a silencing RNA) for human or animal therapy; and
b) P2 comprises (i) a crRNA (eg, comprised by a single guide RNA) that is operable in the host cell for guiding a Cas nuclease to bind to a protospacer sequence comprised by the nucleic acid for cutting of the protospacer, optionally wherein the nucleic acid is degraded, thereby downregulating the expression of P1 or (ii) a precursor of such an crRNA (eg, pre-cRNA).

Human or animal therapy herein may be treatment or prophylaxis of a disease or condition in the human or animal.

For any Configuration herein, a crRNA may be comprised by a guide RNA, such as a single guide RNA. For example, the single guide RNA comprises a crRNA and a tracrRNA that are operable in the recipient and/or carrier (donor) cell with a Cas9 to cut a cognate target nucleic acid sequence.

The invention provides:

A vector according to any Configuration herein for use as a medicament.

The medicament may be for treating or preventing a disease in a human or animal subject, eg, when comprised by a formulation for oral-administration to the subject.

A pharmaceutical composition comprising a vector according to any Configuration herein and a pharmaceutically-acceptable carrier, diluent or excipient, optionally an antacid.

A tablet, suppository, pill. capsule, or liquid formulation for administration to the gastrointestinal tract of a human or animal subject, wherein the table, suppository, pill, capsule or liquid formulation comprises a vector according to any Configuration herein.

Optionally, the tablet pill, or capsule comprises an enteric coating. Optionally, the tablet, pill, capsule or liquid formulation is for use as an orally-administered medicament.

In an example, the composition. tablet, suppository, pill, capsule or formulation herein comprises a medicament selected from the medicaments listed in Table 3.

The invention provides:

A method of temporally regulating the production of an expression product in a human or animal subject, the method comprising
a) administering to a microbiota (eg, a gut microbiota) of the subject a vector comprising a nucleic acid, wherein the microbiota comprises a host cell (eg, bacterial cell) and the nucleic acid encodes a product of interest (P1); optionally wherein the administering is oral or topical administration;
b) allowing transfer of the nucleic acid into the host cell comprised by the microbiota and expression of P1 in the host cell;
c) after step (b) exposing the microbiota to a regulator agent (R) that upregulates production of an RNA-guided nuclease/guide RNA complex in the host cell that is capable of targeting a protospacer comprised by the nucleic acid, wherein the nuclease cuts the nucleic acid and expression of P1 is rendered non-functional (eg, by degradation of the cut nucleic acid in the cell), wherein the nuclease (or a component thereof) and/or RNA (or a component thereof) is encoded by the nucleic acid.

The invention provides:

A method of temporally regulating the production of an expression product in a human or animal subject, the method comprising
a) administering to a microbiota (eg, a gut microbiota) of the subject a vector, composition, tablet, suppository, pill, capsule, or liquid formulation according to any preceding claim; optionally wherein the administering is oral or topical administration;
b) allowing transfer of the nucleic acid into a host cell comprised by the microbiota and expression of P1 in the host cell; and
c) after step (b) exposing the microbiota to R (eg, by administering R to the subject), wherein R regulates the second promoter, thereby regulating the expression of P2 and P1.

Step (b) may be for a desired time between time points T1 and T2. For example for any of the methods, step (a) of the method is commenced at a first time (T1) and step (c) at a second time (T2). Optionally, T2 is at least 0.5, 1, 2, 3, 4, 5, 6, 7, 8, 9, 10, 11, 12, 13, 14, 15, 16, 17, 18 19, 20, 21, 22, 23 or 24 hours, or 2, 3, 4, 5, 6, 7, 8, 9, 10, 11, 12, 13 or 14 days, or 3, 4, 5, 6, 7, or 8 weeks after T1. Preferably, T2 is at least 0.5 hours after T1. Preferably, T2 is at least 1 day after T1. Preferably, T2 is at least 1 week after T1. Preferably, T2 is at least 1 month after T1. Preferably, T2 is at least 2 months after T1. Preferably, T2 is at least 3 months after T1. Preferably, T2 is at least 3 months after T1. Preferably, T2 is at least 4 months after Ti. Preferably, T2 is at least 5 months after TI. Preferably, T2 is at least 6 months after T1. Preferably, T2 is at least 12 months after T1. Preferably, T2 is at least 18 months after T1. For example, T2 is 1-6 (eg, 1, 2, 3, 4, 5 or 6) months after T1. For example, T2 is 1-8 weeks after TI. Thus, in this way the method is a method of temporally regulating the expression.

There is provided:

A method of
i. treating or preventing a disease or condition in a human or animal subject by temporally regulating the production of P1 according to the method of the invention; or
ii. modifying a microbiota (eg, a gut microbiota) of a human or animal subject by temporally regulating the production of P1 according to the method of the invention.

For example, there is provided a method of modifying a metabolome of a human or animal subject by carrying out the method of the invention. For example, expression of P1 causes the secreting or sequestering of one or more metabolites in the subject (eg, in the target or cell comprising the vector nucleic acid). For example, expression of P1 causes altering of a pathway intracellularly in the microbiota of the subject (eg, in the target cell) to cause compound metabolism (e.g. for producing a tryptophan sink, such as wherein the tryptophan is used in the microbiota (eg, in the target cell) for the production of AhR (Aryl Hydrocarbon Receptor) ligands). For example, expression of P1 causes the metabolizing or modifying of a chemical, such as a therapeutic drugs, in the microbiota. P1 may do such “causing” since it is a component of a metabolic pathway in the microbiota (eg, in the target cell), such as wherein P1 is a protein, like an enzyme.

The disease or condition may be any disease or condition described herein. The microbiota of a human or animal may be any microbiota described herein.

The administration of step (a) may be oral, topical (eg, by application on skin), buccal, rectal, vaginal, parenteral, intravenously, intramuscularly, inhaled, subcutaneously, ocular or intranasal administration. Preferably oral administration is used. Preferably topical administration is used.

In an example, the vector is comprised by a Faecal Microbial Transplat (FMT). The administration of step (a) may be by rectal administration of an enema or FMT comprising the vector.

In step (b) the vector (eg, a plasmid) comprising the nucleic acid can be transferred.

P1 may be a therapeutically or prophylactically useful expression product in the subject.

In an embodiment, n step (c) P1 expressed from the nucleic acid is the regulator agent (R) or is a component of a pathway that produces R, whereby a P1 expression feedback loop negatively regulates further expression of P1. For example, Pt is a metabolism pathway enzyme. For example, P1 is an intracellular enzyme in the target cell. For example, P1 is a secreted enzyme (eg, secreted from the target cell). In an example, expression of P1 causes the production (eg, in the microbiota, eg, in the target cell) of R.

A pathway as mentioned herein may be in the target cell or it may be outside the target cell. For example, the pathway is a pathway inside a different cell comprised by the microbiota of which the target cell is a component. The different cell may be a carrier cell (ie, donor cell) or it may be an endogenous cell of the microbiota (ie, any cell of the microbiome except the target cell or the carrier cell).

In an example, R upregulates the second promoter in step (c) and P2 downregulates the expression of P1; and optionally wherein the upregulation of the second promoter causes the production of a guided nuclease or restriction endonuclease that cuts the nucleic acid in the host cell, wherein the nucleic acid is degraded, thereby downregulating the expression of P1.

In an example, P2 is capable of upregulating the expression of P1 in the host cell, wherein R downregulates the second promoter in step (c) whereby the expression of P1 is downregulated.

Downregulation of P1 in step (c) may be by at least 10, 20, 30, 40, 50, 60, 70, 80, 90, or 95% compared to P1 expression in step (b). For example, downregulation of P1 in step (c) may be expression of P1 for a period (PD1) in the presence of R that is at least 10, 20, 30, 40, 50, 60, 70, 80, 90, or 95% less compared to P1 expression in step (b) for a period (PD2), wherein PD1 and PD2 are the same length of time. For example, PD1 and PD2 each is 1 minute, 1 hour, 1 day, 1 week, 1 month, 6 months or 12 months. Expression of P1 may be determined in steps (b) and (c) by assessing P1 expression in a sample of the microbiota taken during steps (b) and (c) respectively. For example, when the microbiota is a gut microbiota of a human or animal subject, each sample may be a faecal sample of the subject.

Any upregulation of P1 expression may be an increase in P1 by at least 10, 20, 30, 40, 50, 60, 70, 80, 90. or 95%, such as compared to P1 expression immediately prior step (b). Any downregulation of P1 expression may be an decrease in P1 by at least 10, 20, 30, 40, 50, 60, 70, 80, 90, or 95%, such as compared to P1 expression immediately prior step (b).

Any upregulation of P2 expression may be an increase in P2 by at least 10, 20, 30, 40, 50, 60, 70, 80, 90, or 95%, such as compared to P2 expression immediately prior step (c). Any downregulation of P2 expression may be an decrease in P2 by at least 10, 20, 30, 40, 50, 60, 70, 80, 90, or 95%, such as compared to P2 expression immediately prior step (c). The vector, composition, tablet, suppository, pill, capsule, or liquid formulation described herein may be for use in the method described herein, such as wherein the vector, composition, suppository, pill, capsule or formulation is administered to the subject by oral or topical administration.

The host, carrier and/or target cells may be bacterial cells. In an alternative, instead of bacterial cells, the host, carrier and/or target cells may be archaea.

In an example, the method modifies the genome of the cell, eg, modifies a chromosome or episome (eg, a plasmid) of the host cell. For example, following transfer of the vector nucleic acid into the host cell, a copy of NS1 is inserted into a chromosome or episome of the host cell. In an example, the expression of a nuclease (eg, P2 or a nuclease that is operable with P2, such as wherein P2 comprises guide RNA) leads to cutting of the NS1 in the chromosome or episome and optionally host cell death.

The carrier cell and target cell may be cells of the same order, family or genus, such as shown in the Examples.

Preferably, the agent comprises a CRISPR/Cas system or component thereof. The agent may be a crRNA or guide RNA that guides a Cas nuclease in the target cell to a target protospacer sequence, wherein the Cas cuts the target sequence and the target cell is killed. For example, the plasmid may encode a plurality of different crRNAs or guide RNAs, such as a first cRNA or gRNA that comprises a spacer sequence that is capable of guiding a Cas in the target cell to a first protospacer sequence and a second cRNA or gRNA that comprises a spacer sequence that is capable of guiding a Cas in the target cell to a second protospacer sequence wherein the protospacer sequences are different (eg, different chromosomal sequences of the target cell). Each protospacer may be comprised by an essential gene, virulence gene or antibiotic resistance gene of the target cell genome. Each protospacer sequence may be from 10 to 60 nucleotides in length, eg, 15 to 50, 15 to 40, 15 to 30 or 15 to 20 nucleotides in length. The target sequence may be a chromosomal sequence of the target cell. The target sequence may be an episomal sequence of the target cell. The plasmid may encode a or said Cas nuclease, optionally a Cas9, Cas3 or Cpf1.

For example, the target cell is comprised by a plant microbiota. The carrier cell may be a Pseudomonas cell, optionally a P fluorescens cell. Optionally, the carrier and target cells are cells of the same genus or species, optionally both are Pseudomonas cells. For example, the target cell is a P syringae or aeruginosa cell and the carrier is a Pseudomonas (eg, P fluorescens) cell.

Preferably, the carrier cells are of a strain or species that is not pathogenic to an organism (eg, a plant, animal or human) that comprises the target cells. The carrier cells may be of a strain or species that is symbiotic or probiotic to an organism (eg, a plant, animal or human) that comprises the target cells, eg, probiotic or symbiotic in the gut of the organism.

For example, the target cell is comprised by a plant microbiota. In an example, the carrier cell comprises a Chitinase class I exoenzyme and/or the carrier cell genome encodes a Chitinase class I exoenzyme. Optionally, the carrier cell in this example is a Pseudomonas, eg, P fluorescens, cell. In an example, the carrier cell comprises a pep1 gene. Optionally, the carrier cell in this example is a Pseudomonas, eg, P fluorescens, cell.

In an example, the carrier cell is a motile bacterial cell. Optionally, the target cell is comprised by a plant microbiota and the carrier cell in this example is a Pseudomonas, eg, P fluorescens, cell.

For example, each target cell is a lag phase cell, exponential phase cell or a stationary phase cell. For example, each carrier cell is a lag phase cell, exponential phase cell or a stationary phase cell.

For example, the target cell is comprised by a plant microbiota. Optionally, the target cell is a Pseudomonas (optionally a P fluorescens or P aeruginosa) cell, Erwinia (optionally E carotovora), Xanthomonas, Agrobacterium, Burkholdi, Clavibacterium, Enterobacteria, Pantoae, Pectobacterium (eg. P atrosepticum). Rhizobium, Streptomyces (eg, S scabies), Xylella (eg, X fastidiosa), Candidatus (eg, C liberibacter), Phytoplasma, Ralstonia (eg, R solanacearum). or Dickeya (eg, D dadantii) cell.

Each target cell (eg, the plurality of target cells) may be a cell of a genus or species disclosed in Table 1 or 2. Each target cell (eg, the plurality of target cells) may be comprised by a plant or a plant environment (such as soil) and selected from a genus or species disclosed in Table 1. Each carrier cell (eg, the plurality of carrier cells) may be a cell of a genus or species disclosed in Table 1 or 2.

The method may be carried out in vitro or ex vivo.

The target cell may be comprised by
(a) a plant microbiota (eg, a microbiota of any plant part disclosed herein),
(b) an animal or human microbiota (eg, a microbiota of any human or animal organ or tissue or part disclosed herein); or
(c) a soil, manure, food or beverage microbiota.

For example, the target cell is comprised by a plant leaf, stem, root, seed, bulb, flower or fruit microbiota.

Optionally, a microbiota herein is a gut, lung, kidney, urethral, bladder. blood, vaginal, eye, ear, nose, penile, bowel, liver, heart, tongue, hair or skin microbiota.

For example, the target cell is a cell of a species found in soil.

The method may be carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed or growth or proliferation of cells of the second population is inhibited (eg, by at least 50, 60, 70, 80, 90 or 95%, such as after 5 or 10 hours or 1 day after commencing the method). Preferably cells of the second population are killed.

Optionally, the method of the Second and Third Configurations is carried out on a plurality of target cells comprised by the microbiota by exposing the plurality of target cells to a plurality of the vectors (eg, a plurality of copies of a vector comprising NS1 and NS2; or a plurality of copies of a first vector comprising NS1 with a plurality of copies of a second vector comprising NS2). The method optionally modifies at least 99%, 99.9%, 99.99%, 99.999%, 99.9999% or 99.99999% cells of said plurality of target cells so that they are capable of expressing P1. In an example, the method is carried out on a population (or said plurality) of said target cells and the method modifies all (or essentially all) of the cells of said population (or said plurality). In an example, the method is carried out on a population (or said plurality) of said target cells and the method modifies 100% (or about 100%) of the cells of said population (or plurality).

Preferably, at least 90, 91, 92, 93, 94, 95, 96, 97, 98 or 99% of the target cells are modified.

Optionally, the target cells are Pseudomonas (eg, P syringae) cells, eg, wherein the cells are comprised by a crop plant, such as a tomato plant. For example, leaf, fruit, ear, seed, grain, head, pod, stem, trunk, tuber and/or root biomass is increased by the method. For example, leaf or fruit dry biomass, leaf or fruit wet biomass or number of flowers is increased by the method, eg, wherein expression of P1 is beneficial to the health or growth of the plant. For example, average biomass or number is increased over a plurality of plants on which the method of the invention has been practised.

An increase in biomass (eg, average biomass or number) may be an increase by at least 5, 10, 15, 20, 25, 30, 40, or 50% compared to the biomass of plant(s) that have not been exposed to the carrier bacteria, but which comprise the target bacteria. Increases in plant biomass may be determined by measuring the weight of harvested material (eg, fruit, grain, cane, leaves, tubers, nuts or seeds) per area harvested and comparing the measurement of harvested material from plants that have been treated per the invention versus the same area of harvested material from plants of the same species and strain grown that have not been treated per the invention, where all plants are grown under the same conditions, eg, in the same field. In some systems units of volume, such as bushels, are used instead of units of weight.

In an Aspect the method is a method of promoting growth of a plant or germination of a plant seed, wherein the method is carried out using a first cell population comprising a plurality of carrier cells that are contacted with a second cell population comprising a plurality of target cells, wherein copies of said nucleic acid are transferred from carrier cells into target cells, whereby some or all of the cells of the second population are modified to express P1, wherein the seed comprises said target cells and said growth or germination is promoted.

Promoting germination may be decreasing the time to onset of germination and/or decreasing the duration of germination. Promoting germination may be increasing the percentage (eg, by at least 5, 10, 15 or 20%) of germination of seeds comprised by a plurality of seeds that are exposed to the carrier cells in the method.

Each seed may comprise target cells on the seed surface.

An increase in germination (eg, average germination) in a plurality of seeds exposed to the carrier cells in the method may be obtained, which is an increase by at least 5, 10, 15, 20, 25, 30, 40, or 50% compared to the germination of seeds that have not been exposed to the carrier cells, but which seeds comprise the target bacteria.

The method may be useful for treating pre-emergent seedlings have pathogens present which stop successful germination. Each seedling may comprise target cells on leaves and/or stems of the seedling.

An increase in growth (eg, average growth) in a plurality of seedlings exposed to the carrier cells in the method may be obtained, which is an increase by at least 5, 10, 15, 20, 25, 30, 40, or 50% compared to the growth of seedlings that have not been exposed to the carrier cells, but which seedlings comprise the target bacteria.

In an Aspect the method is a method of increasing leaf chlorophyll (eg, chlorophyll a and/or b) production in a plant, wherein the method is carried out using a first cell population comprising a plurality of carrier cells that are contacted with a second cell population comprising a plurality of target cells, wherein copies of said nucleic acid are transferred from carrier cells into target cells, whereby some or all of the cells of the second population are modified to express P1, wherein the plant comprises said target cells (optionally on leaves and/or stems thereof, or comprised by the apoplast of the plant), whereby target cells are modified and chlorophyll is increased in the plant. Chlorophyll measurement may be measured, for example, by spectrophotometry, high performance liquid chromatography (HPLC) or fluorometry.

In an Aspect the method is a method of modifying target cells comprised by a biofilm, wherein the biofilm is comprised by a subject or comprised on a surface, wherein the biofilm comprises target cells, wherein the method is carried out using a first cell population comprising a plurality of carrier cells that are contacted with a second cell population comprising a plurality of target cells, wherein copies of said nucleic acid are transferred from carrier cells into target cells, thereby modifying the target cells in the biofilm to express P1, optionally wherein the method is carried out ex vivo or in vitro.

The subject may be a human or animal, optionally wherein the surface is a lung surface.

The subject may be a plant, optionally wherein the biofilm is comprised by a leaf, trunk, root or stem of the plant.

The surface may be comprised by a domestic or industrial apparatus or container, eg, a fermentation vessel.

There is further provided:—

A carrier bacterial cell (eg, for use in a method according to the invention) for administration to a microbiota comprising a target cell, wherein the carrier cell comprises a conjugative plasmid, the plasmid being a vector of the invention, wherein the carrier cell is capable of conjugating to the target cell wherein the plasmid is transferred into the target cell to modify the target cell to express P1.

The carrier cell may be any carrier cell or carrier cell disclosed herein. The target cell may be any carrier cell or target cell disclosed herein.

There is provided:—

A carrier cell (eg, a bacterial cell) comprising a vector of the invention. The invention also provides a plurality of such carrier cells (eg, wherein the cells are genetically identical or wherein all of the cells encode the same P1). In an alternative, cells of the plurality encode different P1 proteins.

A pharmaceutical composition comprising a plurality of carrier cells of the invention for administration to a human or animal subject for modifying a plurality of bacterial target cells comprised by the subject to express P1, wherein vectors of the invention (eg, conjugative plasmids) encoding P1 are capable of being introduced from carrier cells into target cells (eg, by conjugation) and P1 is produced in target cells.

Preferably, at least 90, 91, 92, 93, 94, 95, 96, 97, 98 or 99% of the target cells are so modified.

The plurality of target cells may comprise at least 10⁷, 10⁸, 10⁹, 10¹⁰, 10¹¹ or 10¹² target cells. For example, the plurality of target cells is comprised by a gut, blood, lung, oral cavity, liver, kidney, bladder, urethra or skin microbiota of the subject.

There is provided:—

A method of treating or preventing a disease or condition in a subject, the method comprising contacting the subject (eg, a gut microbiota where the subject is a human or animal) with a composition comprising a plurality of carrier cells of the invention, wherein vectors of the invention (eg, conjugative plasmids) encoding P1 are introduced from carrier cells into target cells (eg, by conjugation) and P1 is produced in target cells, whereby the disease or condition is treated or prevented.

Use of a plurality of carrier cells of the invention in the manufacture of a composition for administration to a subject or environment (eg, soil), for modifying bacterial target cells comprised by the subject or environment to express P1, wherein the target cells are contacted with the carrier cells and vectors of the invention (eg, conjugative plasmids) encoding P1 are introduced from carrier cells into target cells (eg, by conjugation) and P1 is produced in target cells.

For example, the subject is a human or animal. For example, the subject is a mammal. For example, the subject is a bird, fish, protozoan or insect. For example, the animal is a livestock anima. For example, the animal is a dog, cat, horse, cow, sheep or pig.

For example, the subject is a plant and optionally the method comprises contacting the plant (eg, one or more stems and/or one or more leaves of the plant, or the plant apoplast) with the composition comprising a plurality of carrier cells.

There is provided:

Use of a carrier cell of the invention in the manufacture of a composition, for modifying a bacterial target cell ex vivo or wherein the target cell is not comprised by a human or animal (eg, the target cell is comprised by a plant or soil or a human microbiota sample ex vivo), wherein the target cell is contacted with the carrier cell and the carrier cell conjugates to the target cell, whereby the vector nucleic acid is introduced into the target cell, wherein P1 is expressed in the target cell.

For example, each vector is a conjugative plasmid, wherein the carrier cell conjugates (or is capable of conjugating) with the target cell and transfers the plasmid into the target cell by conjugation. Optionally, the plasmid comprises an origin or transer (oriT) and genes for self-conjugation, whereby the plasmid is capable of conjugative transfer from the target cell to a further cell (eg, wherein the target and further cells are comprised by a microbiota).

Optionally, the use comprises using a plurality of said carrier cells to modify a plurality of said target cells, wherein the target cells are comprised by a plant or plant environment (eg, soil) and the modifying
a) increases (or is for increasing) the biomass of the plant or part thereof (eg, leaf, fruit, ear, seed, grain, head, pod, stem, trunk, tuber and/or root biomass is increased);
b) promotes (or is for promoting) germination of one or more seeds of the plant;
c) increases (or is for increasing) the amount of leaf chlorophyll of the plant; and/or
d) reduces (or is for reducing) a biofilm comprised by the plant, wherein the biofilm comprises target cells (eg, Pseudomonas cells).

Optionally, the target cell or plurality of target cells is in an environment, eg, soil, or in an environment for growing plants. For example, P1 is a plant growth promoter (eg, a fertilizer).

### Example Target Cells

For example, each target cell is a Bacteroides cell, eg, comprised by a human or animal subject. For example, each target cell is a Clostridiales cell, eg, comprised by a human or animal subject.

For example, each target cell is a gram-positive bacterial cell (eg, a Staphylococcus (such as S aureus, eg, methicillin-resistant Staphylococcus aureus (MRSA)), Streptococcus pneumonia, Clostridium difficile. Enterococcus spp. or Listeria monocytogenes cell). For example, each target cell is a gram-negative bacterial cell (eg, a Acinetobacter baumannii, Escherichia coli, Klebsiella pneumoniae, Stenotrophomonas maltophilia, Campylobacter jejuni, Pseudonmoas aeruginosa, Neisseria gonorrhoeae. Vibrio cholerae or Salmonella spp. Cell) For example, each target cell is a cell of a genus or species disclosed in Table 1 herein or Table 2 herein.

Reference is made to Journal of Plant Pathology (2010), 92 (3), 551-592 Edizioni ETS Pisa, 2010 551, LETTER TO THE EDITOR. “COMPREHENSIVE LIST OF NAMES OF PLANT PATHOGENIC BACTERIA, 1980-2007”, C. T. Bull et al, the disclosure of which is incorporated herein by reference to provide examples of bacterial genera, species and strains of importance to plants and which may be genera, species and strains of target cells of the invention. Examples are disclosed in Table 1 herein.

For example, each target cell is resistant to a fluoroquinolone. β-lactam (eg, methicillin), tetracycline or linezolid antibiotic. For example, each target cell is resistant to vancomycin, eg, wherein the cell is a vancomycin-resistant Enterococcus cell.

For example, each target cell is an Azotobacter, Burkwideria, Cupriavidus, Enterococcus, Lysobacter, Paucimonas, Paraburkholderia, Ralstonia, Stenotrophomonas, Variovorax, Xanthomonas or Pseudomonas cell, eg, wherein the target cell is comprised by a plant.

For example, each target cell is an E coli cell.

For example, each target cell is Klebsiella cell, eg, wherein the target cell is comprised by a plant.

For example, each target cell is an Azotobacter, Burkholderia, Cupriavidus. Lysobacter, Paraburkholderia, Ralstonia, Variovorax, Xanthomonas or Pseudomonas cell, eg, wherein the target cell is comprised by a plant.

For example, each target cell is a cell of a Pseudomonas species, optionally wherein the species is selected from Pseudomonas aeruginosa Pseudomonas anmygdali, Pseudomonas asturiensis, Pseudomonas avellanae, Pseudomonas cerasi, Pseudomonas chlororaphis, Pseudomonas cichorii, Pseudomonas coronafaciens, Pseudomonas otitidis, Pseudomonas putida, Pseudomonas salegens Pseudomonas savastanoi, Pseudomonas syringae and Pseudomonas viridiflava, eg, wherein the target cell is comprised by a plant.

For example, each target cell is a cell of a species selected from Azotobacter chroococcum, Azotobacter salinestris, Burkholderia ambifaria, Burkholderia cenocepacia, Burkholderia lata, Burkholderia pyrrocinia, Cupriavidus basilensis, Cupriavidus necator, Cupriavidus taiwanensis, Lysobacter gunmosus, Paraburkholderia sprentiae, Paraburkholderia terricola, Ralstonia pseudosolanacearum, Ralstonia solanacearum, Variovorax paradoxus, Xanthomonas arboricola, Xanthomonas axonopodis, Xanthomonas campestris Xanthomonas citri, Xanthomonas euvesicatoria and Xanthomonas perforans, eg, wherein the target cell is comprised by a plant.

For example, each target cell is a Stenotrophomonas, Enterococcus, Paucimonas or Pseudomonas cell, eg, wherein the target cell is comprised by a plant.

For example, each target cell is a cell of a Pseudomonas species, optionally wherein the species is selected from Pseudomonas amygdali. Pseudomonas asturiensis, Pseudomonas avellanae. Pseudomonas cerasi, Pseudomonas chlororaphis, Pseudomonas cichorii, Pseudomonas coronafaciens, Pseudomonas putida, Pseudomonas savastanoi, Pseudomonas syringae and Pseudomonas viridiflava, eg, wherein the target cell is comprised by a plant.

For example, each target cell is a cell of a species selected from Stenotrophomonas rhizophila. Enterococcus faecalis, Paucimonas lemoignei, Pseudomonas amygdali, Pseudomonas asturiensis, Pseudomonas avellanae, Pseudomonas cerasi, Pseudomonas chlororaphis, Pseudomonas cichorii, Pseudomonas coronafaciens, Pseudomonas putida, Pseudomonas savastanoi, Pseudomonas syringae and Pseudomonas viridiflava, eg, wherein the target cell is comprised by a plant.

### Example Carrier Cells

For example, the carrier is an E coli cell (eg, E coli, K12, Nissle or S17 cell). eg, wherein the cell is for administration to a human or animal subject, such as to treat or prevent a disease or condition. For example, each carrier cell is an Bacteroides cell, eg, wherein the cell is for administration to a human or animal subject, such as to treat or prevent a disease or condition. For example, each carrier cell is an Clostridiales cell, eg, wherein the cell is for administration to a human or animal subject, such as to treat or prevent a disease or condition.

For example, each carrier cell is a gram-positive bacterial cell. For example, each carrier cell is a gram-negative bacterial cell. For example, the carrier cell is a cell of a genus or species disclosed in Table 1 of WO2017211753 (the disclosure of this table and each genus and species individually being incorporated herein for disclosure of cell genus or species that may be used in the present invention).

For example, the carrier cell is a cell of phylum Proteobacteria, class Gammaproteobacteria, order Pseudomonadales or family Pseudomonadaceae. In a preferred example, the carrier is a Pseudomonas (eg, P fluorscens) cell, eg, wherein the target cell is comprised by a plant.

For example, each carrier cell is Klebsiella cell, eg, wherein the target cell is comprised by a plant.

For example, the carrier is a gram positive cell. eg, a Bacillus (such as Bacillus subtilis) or Cloistridiales (such as Clostridium butyricum) cell.

In an example, the subject is a shellfish. The shellfish may be selected from shrimp, crayfish, crab, lobster, clam, scallop, oyster, prawn and mussel.

The subject may be any subject disclosed herein. The subject may be an animal, such as a livestock animal, eg, a bird (such as a poultry bird; or a chicken or a turkey) or swine.

In an alternative, the subject is a plant, eg, and the target bacteria are plant pathogen bacteria. In an example, the target bacteria are Pseudomonas, eg, P syringae or P aeruginosa.

In an alternative, the carrier and target cells are archaeal cells. For example the target cells are methanobacterium cells. For example the target cells are methanogen cells. For example, the target cells comprise one or more species of cell selected from:
Methanobacterium bryantii
Methanobacterium formicum
Methanobrevibacter arboriphilicus
Methanobrevibacter goltsclalkii
Methanobrevibacter rumninantium
Methanobrevibacter smithii
Methanococcus chunghtsingensis
Methanococcus burtonii
Methanococcus aeolicas
Methanococcus deltae
Methanococcus jannaschii
Methanococcus naripaludis
Methanococcus vannielii
Methanocorpusculum labreanum
Methanoculleus bourgensis (Methanogenium olentangyi & Methanogenium bourgense)
Methanoculieus narisnigri
Methanoflorens stordalenmirensis
Methanofollis liminatans
Methanogenium cariaci
Methanogenium frigidumn
Methanogeniuna organophilum
Methanogenium wolfei
Methanomicrobium mobile
Methanopyrus kandleri
Methanoregula boonei
Methanosaeta concilii
Methanosaeta thermophila
Methanosarcina acetivorans
Methanosarcina barkeri
Methanosarcina mazei
Methanosphaera stadtmanae
Methanospirillium hungatei
Methanothermobacter defluvii (Methanobacterium defluvii)
Methanothermobacter themauotrophicuts (Methanobacterium thernoautotrophicum)
Methanothernwbacter thernoflexus (Methanobacteritn thermoflexwn)
Methanothermobacter wolfei (Methanobacterium wolfei)
Methanothrix sochngenii

Optionally, the target cells are not pathogenic to the subject, for example when the method is a non-medical method. In an example, the method is a cosmetic method.

In the example, optionally the subject or animal is a livestock animal, such as a cow, sheep. goat or chicken (preferably a cow). Optionally, eg, wherein the subject is an animal (eg, a livestock animal or a wild animal), the target cells are zoonotic bacterial cells, such as cells of a species selected from Bacillus anthracis, Mycobacterium bovis (eg, wherein the animal is a cow), Campylobacter spp (eg. wherein the animal is a poultry animal), Mycobacterium marinum (eg. wherein the animal is a fish), Shiga toxin-producing E. coli (eg. wherein the animal is a ruminant), Listeria spp (eg, wherein the animal is a cow or sheep), Chlamydia abortus (eg, wherein the animal is a sheep), Coxiella burnetii (eg, wherein the animal is a cow, sheep or goat), Salmonella spp (eg, wherein the animal is a poultry animal), Streptococcus suis (eg, wherein the animal is a pig) and Corynebacterium (eg, C ulcerans) (eg, wherein the animal is a cow).

In an example, a plurality of carrier cells as described herein (eg, carrier cells of any configuration, aspect, example or embodiment described herein) is administered to the subject, wherein the carrier cells comprise the nucleic acid encoding P1.

In an example, each animal is a chicken. In an example, each animal is a cow (eg, a beef or dairy cow).

Optionally the method modifies target cells in the gastrointestinal tract of the subject human or animal; optionally the method modifies target cells in the jejunum, ileum, colon, liver, spleen or caecum of the subject: optionally wherein the animal is a bird and the method modifies target cells in the caecum of the bird. In an example the method is carried out on a group (optionally a flock or herd) of animals, wherein some or all of the animals comprise target cells.

Optionally, the plasmid comprises a RP4 origin of transfer (oriT). The plasmid may be any type of plasmid disclosed herein.

### P2

P2 may be any antibacterial agent disclosed herein or a component thereof, preferably a guided nuclease that is programmed to cut one or more target sequences in target cells. A suitable nuclease may be a TALEN, meganuclease, zinc finger nuclease or Cas nuclease. For example, the agent comprises one or more components (eg, a Cas nuclease and/or a guide RNA or a crRNA) of a CRTSPR/Cas system that is operable in a target cell to cut a protospacer sequence comprised by the target cell (eg, comprised by the vector, such that cut vector is degraded in the target cell). For example, the system is operable to cut at least 2 or 3 different protospacer sequences comprised by the vector of the invention. Optionally, P2 is operable to cut a plurality of different protospacer sequences comprised by the vector (and optionally further operable to cut the carrier cell genome, eg, a carrier cell chromosomal or episomal protospacer sequence, wherein cutting thereof is lethal to the carrier cell—as explained elsewhere, this is useful to reduce or remove carrier cells from the subject or microbiota when desired, such as after a desired level of P1 expression in target cells has been obtained). Optionally, the agent comprises one or more components of a CRISPR/Cas system that is operable to cut at least 2, 3, 4, 5, 6, 7, 8, 9, or 10 different protospacer sequences comprised by the vector and/or carrier cell genome (eg, comprised by the carrier cell chromosome).

In an embodiment, P2
(a) comprises a guided nuclease that is capable in a target cell of recognising and cutting a protospacer sequence comprised by the vector; and/or
(b) encodes a guide RNA or crRNA of a CRISPR/Cas system that operates with a Cas nuclease in a target cell to cut a protospacer sequence comprised by the vector;
   wherein the nuclease is capable of cutting the vector in the target cell and the vector is degraded in the target cell.

This is useful as an “off switch” to remove the vector from the target cell, eg, for downregulating the capability of P1 expression in a plurality of target cells that have been previously contacted with carrier cells and into which target cells vectors of the invention have been transferred for P1 expression. For example, this usefully can be used to clear the microbiota of a subject so that it can produce less or no P1 after cutting of vector nucleic acid has taken place. In this way, for example, it is possible in a first step to modify a microbiota of a subject (eg, a human gut microbiota) so that it produces P1 (eg, where P1 expression is useful for treatment or prevention of a disease or condition or alternatively for non-medical utility) and thereafter P1 expression can be reduced or removed and the microbiota can be restored to a partially modified or unmodified condition.

Any administration of cells to a subject herein may be by oral administration. Any administration of cells to a subject herein may preferably be by administration to the GI tract. Any administration of cells to a subject herein may be by systemic, intranasal or inhaled administration.

There is provided the following definitions:—

Homologue: A gene, nucleotide or protein sequence related to a second gene, nucleotide or protein sequence by descent from a common ancestral DNA or protein sequence. The term, homologue, may apply to the relationship between genes separated by the event of or to the relationship between genes separated by the event of genetic duplication.

Orthologue: Orthologues are genes, nucleotide or protein sequences in different species that evolved from a common ancestral gene, nucleotide or protein sequence by speciation. Normally, orthologues retain the same function in the course of evolution.

Optionally, each P2 is a guide RNA. Optionally, each vector (eg, plasmid) encodes a plurality of guide RNAs or crRNAs of a CRISPR/Cas system wherein the guide RNAs or crRNAs are operable with Cas nuclease in the target cell to recognise a plurality of protospacer sequences comprised by the vector and/or carrier cell genome, optionally wherein the protospacer sequences comprise one or more nucleotide sequences of genes selected. In an example, the vector additionally or alternatively encodes a Cas, eg, a Cas9, Cas3, Cpf1, Cas12, Cas13, CasX or CasY.

In an embodiment, a Cas herein is a Type I, H, HI, IV, V or VI Cas, preferably a Type I or II Cas.

In an example, the vector also encodes a Cas3 and cognate Cascade proteins (eg, CasA, B, C, D and E). Optionally, the Cas (and Cascade of present) are E coli Cas (and Cascade).

The plasmid may comprise one or more CRISPR spacers, wherein each spacer consists of 20-40, 25-35, or 30-35 consecutive nucleotides, eg, consecutive nucleotides selected from

Optionally, the plasmid comprises a RP4 origin of transfer (oriT) and/or a p15A origin of replication.

In an example, the plasmid is a conjugative phagemid.

In an example, the vector encodes a Cas3 and optionally one or more Cascade proteins (eg, one or more of CasA, B, C, D and E). In an embodiment, the vector encodes a Cas3 and CasA, B, C, D and E. In an embodiment, the vector encodes an E coli Cas3 and CasA, B, C, D and E. Optionally, the guided nuclease (eg, Cas3) is a Type I-A, -B, -C, -D, -E, -F or -U Cas.

In an example, P2 in any configuration, aspect, example, option or embodiment herein comprises one or more components of a CRISPR/Cas system that is operable in the target cell to cut a protospacer sequence comprised by the vector. Additionally, in an example, P2 in any configuration, aspect, example, option or embodiment herein comprises one or more components of a CRISPR/Cas system (eg, the same system as in the first sentence in this paragraph) that is operable in the carrier cell to cut a protospacer sequence comprised by the carrier cell genome (eg, a chromosomal or episomal sequence whose cutting is lethal to the carrier cell). This is useful to clear the carrier cell from the subject (eg, from a gut microbiota) when expression of P1 is no longer required.

In an example, the system is operable to cut at least 3 different protospacer sequences comprised by the vector or carrier cell genome.

In an example, the vector
(a) encodes a guided nuclease that is capable of recognising and modifying a carrier cell nucleic acid sequence. wherein the sequence is comprised by an endogenous chromosome or episome of the carrier cell, wherein the nuclease cuts the chromosome or episome to kill the carrier cell or inhibit the growth or proliferation of the target cell; and/or
(b) encodes a guide RNA or crRNA of a CRISPR/Cas system that operates with a Cas nuclease in the carrier cell to cut a protospacer sequence comprised by the cell.

The expression of (a) and/or (b) may be inducible by exposure of the carrier cell to a regulator agent, eg, R. For example, exposure of the carrier and target cells to R may induce production of P2, wherein P2 comprises component (a) and/or (b) whereby vector nucleic acid is cut in the target cell and the genome of the carrier cell is also cut. This kills the carrier cell and leads to degradation of the vector in the target cell. This usefully can then clear the subject of the vector and the carrier cell, eg after a desirable amount of P1 has been expressed in the subject.

Optionally, the Cas, Cascade proteins, gRNAs and crRNAs are E. coli K12 (MG1655) Cas, Cascade proteins, gRNAs and crRNAs respectively. Optionally, the vector is devoid of nucleotide sequences encoding Cas1 and Cas2 proteins.

In embodiments, by action of P2 (eg, components (a) and/or (b)) in the carrier cells, growth or proliferation of carrier cells is reduced (eg, by at least 40, 50, 60, 70, 80, or 90% compared to growth in the absence of P2 therein).

For example, the carrier cells may be comprised by a medicament for treating or preventing a disease or condition in a human or animal; a growth promoting agent for administration to animals for promoting growth thereof; killing zoonositic bacteria in the animals; for administration to livestock as a pesticide; a pesticide to be applied to plants; or a plant fertilizer.

An advantage may be that the carrier cells may be used as producer cells in which vectors of the invention can be replicated (eg, before (eg, in vitro) and/or following administration to the subject).

### Example Plasmids

A method of delivery of a vector can be by bacterial conjugation, a natural process whereby a donor bacterium (carrier bacterium) transfers plasmid DNA from itself to a recipient bacterium (target bacterium). Donor bacteria elaborate a surface structure, the pilus which can be considered to be like a syringe or drinking straw through which the DNA is delivered. The donor pilus binds to the surface of a receptive recipient and this event triggers the process of DNA transfer. Plasmids are suitable for this conjugative process, where the plasmid comprises DNA encoding the agent of the invention.

DNA transfer by conjugation may only take place with a ‘susceptible recipient’ but does not generally occur with a recipient carrying a similar type of plasmid. Because conjugation is via pilus bridge, it is possible for that bridge to attach itself not to a recipient but to the donor bacterium. This could result in a futile cycle of transfer of the plasmid DNA to itself. Plasmids thus naturally encode incompatibility factors. One is a surface arrayed protein that prevents the pilus binding to bacterium displaying that surface protein such as itself or any other bacterium carrying the same plasmid. Additionally, plasmids naturally encode another incompatibility system that closely regulates the copy number of the plasmid inside a bacterium. Thus, should a conjugation event manage to evade surface exclusion and start to transfer DNA by conjugation, the recipient will prevent that plasmid establishing as it already maintains the current copy number and will not accept and maintain a further unwanted additional copy.

In an example of the invention, the plasmid is a member of a plasmid incompatibility group, wherein the target cell does not comprise a plasmid of said group. Optionally. the plasmid of the invention is a member of the incompatibility group P (ie, the plasmid is an incP plasmid). For example within the Enterobacteriaceae the following is a non-exclusive list of potential plasmids that could use for delivery: IncFI, IncFII, IncFIll, IncFIV, IncFV, IncM, Inc9, InclO, Incl, IncA, IncB, IncC, IncH, IncIa, InclIc, Incl2, Incly, IncJ, InwL, IncN, Inc2e, IncO, IncP, IncS, IncT and/or IncW. Thus, optionally, the target cell is an Enterobacteriaceae cell and the vector of the invention is a plasmid, wherein the plasmid is selected from an IncFI, IncFII, IncFIll, IncFIV, IncFV, IncM, Inc9, InclO, Incl, IncA, IncB, IncC, IncH, Incla, InclIc, IncII, Incl2, IncIy, IncJ, IncL, IncN, Inc2e, IncO, IncP, IncS, IncT and IncW plasmid.

Preferably, the subject is a human or animal and the plasmid is an IncI plasmid, eg, an IncIH or IncI2 plasmid.

In an example, the carrier cell of the invention comprises two or more plasmids, each plasmid comprising a DNA that encodes a respective P1 and P2 (P1/P2 may be the same in the cells or the cells may comprise different P1 and/or P2). Optionally, a first of said plasmids is a member of a first incompatibility group, wherein the target cell does not comprise a plasmid of said first group, and wherein a second of said plasmids is a member of a second incompatibility group, wherein the target cell does not comprise a plasmid of said second group. For example, a carrier cell may comprise an incP plasmid encoding P1 and P2 (eg, a CRISPR-Cas system or a component thereof (eg, encoding a first crRNA or guide RNA that targets a first protospacer sequence of the vector)) and wherein the carrier cell further comprises an incF1 plasmid (eg, encoding P1 and P2 (eg, an anti-carrier cell CRISPR-Cas system or a component thereof (eg, encoding a second crRNA or guide RNA that targets a protospacer sequence of the carrier cell genome)). The protospacers may comprise different nucleotide sequences. Optionally, the carrier cell comprises a group of plasmids comprising 2, 3, 4, 5, 6 or more different types of plasmid, wherein each plasmid is capable of being conjugatively transferred into a target cell, wherein the plasmids encode different P1 and/or P2 products. For example, the plasmids encode different cRNAs or gRNAs that target different protospacers comprised by the vector, carrier cell and/or target cell genome. For example, the group of plasmids comprises up to n different types of plasmid, wherein the plasmids are members of up to n different incompatibility groups, eg, groups selected from IncFI, ItncFlI, IncFIll, IncFIV, hncFV, lncM, lnc9, InclO, Incl, IncA, IncB, IncC, IncH, IncIa, IclIc, IncI2, IncIy, IncJ, IncL, IncN, Inc2e, IncO, IncP, IncS, lncT and IncW. For example, n=2, 3, 4, 5, 6, 7, 8, 9 or 10.

For example, the carrier cell comprises (i) a first vector (eg, plasmid) that encodes a P2 which is a first type of CRISPR/Cas system that targets a first protospacer comprised by the vector, or encodes a component of said system; and (ii) a second vector (eg, plasmid) that encodes a P2 which is a second type of CRISPR/Cas system that targets a second protospacer comprised by the vector or carrier cell genome (eg, a carrier cell chromosomal sequence), or encodes a component of said system, wherein the first and second types are different. For example, the first type is a Type I system, and the second type is a Type H system (eg, the first vector encodes a Cas3, Cascade and a crRNA or guide RNA that is operable with the Cas3 and Cascade in the target cell to modify the first protospacer; and the second vector encodes a Cas9 and a crRNA or guide RNA that is operable with the Cas9 in the target cell to modify the second protospacer). In an alternative, the Cas3 and Cascade are encoded by endogenous target cell genes, wherein the first vector encodes the crRNA or guide RNA that is operable with the endogenous Cas3 and Cascade in the target cell to modify the first protospacer. In an alternative, the Cas9 is encoded by an endogenous target cell gene, wherein the second vector encodes the crRNA or guide RNA that is operable with the endogenous Cas9 in the target cell to modify the second protospacer. Optionally, the Cas3 and Cascade are encoded by endogenous genes of the target cell and the Cas9 is encoded by the second vector.

Instead of a Type I and Type II system, the invention alternatively provides in an embodiment a first vector (eg, plasmid) encoding a Type I CRISPR/Cas system (or component thereof, eg, a Cas3 or a crRNA or a gRNA) and a second vector (eg, plasmid) encoding a Type III CRISPR/Cas system (or a component thereof). Instead of a Type I and Type II system, the invention alternatively provides in an embodiment a first vector encoding a Type I CRISPR/Cas system (or component thereof) and a second vector encoding a Type IV CRISPR/Cas system (or a component thereof). Instead of a Type I and Type II system, the invention alternatively provides in an embodiment a first vector encoding a Type I CRISPR/Cas system (or component thereof) and a second vector encoding a Type V CRISPR/Cas system (or a component thereof). Instead of a Type I and Type II system, the invention alternatively provides in an embodiment a first vector encoding a Type I CRISPR/Cas system (or component thereof) and a second vector encoding a Type VI CRISPR/Cas system (or a component thereof).

Instead of a Type I and Type 11 system, the invention alternatively provides in an embodiment a first vector encoding a Type II CRISPR/Cas system (or component thereof, eg, a Cas9 or a crRNA or a gRNA) and a second vector encoding a Type III CRISPR/Cas system (or a component thereof). Instead of a Type I and Type 11 system, the invention alternatively provides in an embodiment a first vector encoding a Type II CRISPR/Cas system (or component thereof) and a second vector encoding a Type IV CRISPR/Cas system (or a component thereof). Instead of a Type I and Type H system, the invention alternatively provides in an embodiment a first vector encoding a Type H CRISPR/Cas system (or component thereof) and a second vector encoding a Type V CRISPR/Cas system (or a component thereof). Instead of a Type I and Type II system, the invention alternatively provides in an embodiment a first vector encoding a Type II CRISPR/Cas system (or component thereof) and a second vector encoding a Type VI CRISPR/Cas system (or a component thereof).

Instead of a Type I and Type 11 system, the invention alternatively provides in an embodiment a first vector encoding a Type V CRISPR/Cas system (or component thereof, eg, a Cas12a or a crRNA) and a second vector encoding a Type III CRISPR/Cas system (or a component thereof). Instead of a Type I and Type H system, the invention alternatively provides in an embodiment a first vector encoding a Type V CRISPR/Cas system (or component thereof) and a second vector encoding a Type IV CRISPR/Cas system (or a component thereof). Instead of a Type I and Type II system, the invention alternatively provides in an embodiment a first vector encoding a Type V CRISPR/Cas system (or component thereof) and a second vector encoding a Type V CRISPR/Cas system (or a component thereof). Instead of a Type I and Type H system, the invention alternatively provides in an embodiment a first vector encoding a Type V CRISPR/Cas system (or component thereof) and a second vector encoding a Type VI CRISPR/Cas system (or a component thereof).

Instead of a Type I and Type II system, the invention alternatively provides in an embodiment first and second vectors (eg, plasmids), each encoding a Type I CRISPR/Cas system (or a component thereof). Instead of a Type I and Type H system, the invention alternatively provides in an embodiment first and second vectors, each encoding a Type H CRISPR/Cas system (or a component thereof). Instead of a Type I and Type 11 system, the invention alternatively provides in an embodiment first and second vectors, each encoding a Type III CRISPR/Cas system (or a component thereof). Instead of a Type I and Type II system, the invention alternatively provides in an embodiment first and second vectors, each encoding a Type IV CRISPR/Cas system (or a component thereof). Instead of a Type I and Type II system, the invention alternatively provides in an embodiment first and second vectors, each encoding a Type V CRISPR/Cas system (or a component thereof). Instead of a Type I and Type H system, the invention alternatively provides in an embodiment first and second vectors, each encoding a Type VI CRISPR/Cas system (or a component thereof).

Optionally, the plasmids are members of different incompatibility groups, eg, groups selected from IncFI, IncFII, IncFIll, IncFIV, IncFV, IncM, Inc9, InclO, Incl, IncA, IncB, IncC, IncH, IncIa, InclIc, Incll, IncI2, IncIy, IncJ, IncL, IncN, Inc2e, IncO, IncP, IncS, IncT and IncW. In an example here, the target cell is an Enterobacteriaceae cell.

Advantageously, the carrier cells are for treating or preventing a target cell infection in a human or an animal subject (eg, a dog, cat, horse, chicken, cow, sheep, goat, pig, fish or shellfish).

Advantageously, the carrier cells are of a species that is probiotic to said subject or is probiotic to humans or animals (eg, chickens). For example, the carrier cells are probiotic Bacteriodetes (eg, Bacteroides) cell, eg, wherein the subject is a human. For example, the carrier cells are probiotic Clostridiales cell, eg, wherein the subject is a human. For example, the carrier cells are probiotic E coli cell. For example, the carrier cells are probiotic Bacillus cell, eg, wherein the subject is a plant. Advantageously, each vector (eg, virus or plasmid) encodes one or more guide RNAs or one or more crRNAs that are capable of hybridizing in the target cell to respective vector target nucleic acid sequence(s). For example, each vector encodes 2, 3, 4, 5, 6, 7, 7, 9, or 10 (or more than 10) different gRNAs or different crRNAs that hybridise to a respective target sequence, wherein the target sequences are different from each other. For example, 3 different gRNAs or crRNAs are encoded by each vector. For example, 2 different gRNAs or crRNAs are encoded by each vector. For example, 3 different gRNAs or crRNAs are encoded by each vector. For example, 4 different gRNAs or crRNAs are encoded by each vector. For example, 3 different gRNAs or crRNAs are encoded by each vector. For example, 5 different gRNAs or crRNAs are encoded by each vector. For example, 6 different gRNAs or crRNAs are encoded by each vector. For example, 7 different gRNAs or crRNAs are encoded by each vector. For example, 8 different gRNAs or crRNAs are encoded by each vector. For example, 9 different gRNAs or crRNAs are encoded by each vector. For example, 10 different gRNAs or crRNAs are encoded by each vector. For example, 11 different gRNAs or crRNAs are encoded by each vector. For example, 12 different gRNAs or crRNAs are encoded by each vector. For example, 13 different gRNAs or crRNAs are encoded by each vector.

In an example, the target cells are Salmonella cells (eg, wherein the subject is a chicken). In an example, the target cells are Campylobacter cells (eg, wherein the subject is a chicken). In an example, the target cells are Edwardsiella cells (eg, wherein the subject is a fish or shellfish. eg, a catfish or a shrimp or prawn). In an example, the target cells are Bacteriodetes (eg, Bacteroides) cells. In an example the target cells are Clostridiales cells. In an example, the target cells are E coli cells.

Optionally, each plasmid comprises an expressible tra1 and/or tra2 module or a homologue thereof for conjugative transfer of the plasmid between cells. Any episome herein may be a plasmid.

Optionally, each plasmid comprises an expressible operon of a tra1 and/or tra2 module or a homologue thereof for conjugative transfer of the plasmid between cells.

Optionally, each plasmid is a modified RK2 or R6K plasmid. The modification comprises an insertion of nucleotide sequences encoding P1 and P2.

Optionally, each plasmid comprises an oriV, eg, oriV of an IncI (eg, IncI1 or IncI2), RK2 or R6K plasmid, or a homologue thereof. Optionally, each plasmid comprises an oriV of an IncI (eg, IncI1 or IncI2), RK2 or R6K plasmid, or a homologue thereof

Optionally, each plasmid comprises an oriT, eg, oriT of an IncI1 or IncI2 plasmid. Optionally, each plasmid is a modified an IncI (eg, IncI1 or IncI2) plasmid. The modification (for the First to Third Configurations, and optionally for the Fourth Configuration) comprises an insertion of nucleotide sequences encoding P1 and P2.

Optionally, P2 comprises one or more components of a CRISPR/Cas system that is operable in the target cell to cut a protospacer sequence comprised by the vector or carrier cell genome, eg, wherein the protospacer sequence is comprised by the carrier cell chromosome.

In an embodiment, the cutting herein kills the carrier cell or causes degradation of the vector in the target cell. In an alternative, the cutting inhibits the growth or proliferation of the target cell.

Optionally, P2 encodes a guide RNA or crRNA of a CRISPR/Cas system that is operable with a Cas nuclease in the target cell to cut a protospacer sequence comprised by the vector.

In an example of the First to Third Configurations, the protospacer is comprised by a gene required for vector viability or maintenance in the cell. In an example of the Fourth Configuration, the protospacer is comprised by a gene required for carrier cell viability.

Optionally, each vector (eg, virus (eg, phage) or plasmid) comprises a gene that encodes a product, wherein the product is essential for survival or proliferation of the carrier cell when in an environment that is devoid of the product, wherein the carrier cell chromosome does not comprise an expressible gene encoding the product and optionally the vector nucleic acid is the only episomal nucleic acid comprised by the carrier cell that encodes the product. For example, the gene is selected from an aroA, argiH, hisD, leuB, lysA, retB, proC, thrC, pheA, tyrA, trpC and pflA gene; or wherein the gene is an anti-toxin gene and optionally the vector encodes a cognate toxin.

For example, the carrier cell is an E coli (eg, Nissle, F18 or S17 E coli strain) cell. For example, the carrier cell is a Bacillus (eg, B subtilis), Enterococcus or Lactobacillus cell, eg, wherein the subject is a plant.

Optionally, each carrier cell is for administration to a microbiota of a human or animal subject for medical use.

For example, the medical use is for treating or preventing a disease disclosed herein. For example, the medical use is for treating or preventing a condition disclosed herein.

Optionally, the medical use is for the treatment or prevention of a disease or condition mediated by said target cells. Optionally, the medical use is for the treatment or prevention of a disease or condition mediated by cells of a microbiota that also comprises said target cells. For example, P1 is secreted from target cells and acts to kill or modify the growth or metabolism of neighbouring cells in the microbiota.

Optionally, the carrier cell(s) is(are) for administration to a human or animal for enhancing growth or weight of the human or animal. Optionally, the carrier cell(s) is(are) for administration to a human or animal for reducing growth or weight of the human or animal. Optionally, the carrier cell(s) is(are) for administration to a human or animal for reducing obesity in the human or animal.

In an embodiment, the administration is to a human for enhancing the growth or weight of the human. Optionally, the enhancing is not a medical therapy. Optionally, the enhancing is a medical therapy.

Optionally, the use comprises the administration of a plurality of carrier cells to a microbiota (eg, a gut microbiota) of a human or animal subject, wherein the microbiota comprises target cells and the vector nucleic acid is transferred into target cells for expression therein of P1, thereby killing microbiota cells in the subject or reducing the growth or proliferation of microbiota cells.

Optionally, the use comprises the administration of a plurality of carrier cells to a microbiota (eg, a gut microbiota) of a human or animal subject, wherein the microbiota comprises target cells and the vector nucleic acid is transferred into target cells for expression therein of P1, thereby promoting growth or metabolism of microbiota cells in the subject.

Optionally, the use comprises the administration of a plurality of carrier cells to a microbiota (eg, a gut microbiota) of a human or animal subject, wherein the microbiota comprises target cells and the vector nucleic acid is transferred into target cells for expression therein of P1, wherein P1 is a protein (eg, an enzyme) in a metabolic pathway in cells of the microbiota. For example, P1 is secreted by target cells and is taken up by further cells in the microbiota for use of P1 in a metabolic pathway in the further cells.

Optionally, the use comprises the administration of a plurality of carrier cells to a microbiota (eg, a gut microbiota) of a human or animal subject, wherein the microbiota comprises target cells and the vector nucleic acid is transferred into target cells for expression therein of P1, wherein P1 is a protein that is capable of sequestering a substance (eg, a protein, peptide, nucleic acid (eg, RNA), carbohydrate (eg, a sugar or precursor thereof), amino acid, lipid, fatty acid, ion or chemical compound) in the subject. For example, P1 is secreted by target cells and is taken up by further cells in the microbiota for use of P1 as a sequestering agent in the further cells. For example, P1 is secreted by target cells for use of P1 as a sequestering agent in the subject, eg, in the microbiota, an organ, tissue, cell or bloodstream of the subject.

For example, a plant herein in any configuration or embodiment of the invention is selected from a tomato plant, a potato plant, a wheat plant, a corn plant, a maize plant, an apple tree, a bean-producing plant, a pea plant, a beetroot plant, a stone fruit plant, a barley plant, a hop plant and a grass. For example, the plant is a tree, eg, palm, a horse chestnut tree, a pine tree, an oak tree or a hardwood tree.

For example the plant is a plant that produces fruit selected from strawberries, raspberries, blackberries, redcurrants, kiwi fruit, bananas, apples, apricots, avocados, cherries, oranges, clementines, satsumas, grapefruits, plus, dates, figs, limes, lemons, melons, mangos, pears, olives or grapes. Optionally, the plant is a dicotyledon. Optionally, the plant is a flowering plant. Optionally, the plant is a monocotyledon.

In an example, the weight (ie, biomass) of a plant is dry weight. For example, the method is for increasing plant dry weight (eg, within 1 or 2 weeks of said administration). Optionally, the increase is an increase of at least 5, 10, 11, 12, 13, 14, 15, 16, 17, 18, 19 or 20% compared to a control plant of the same species or strain to which the administration if carrier cells has not taken place, wherein all plants are kept under the same environmental conditions. For example, such an increase is within 1, 2, 3, 4, 5, 6, or 8 weeks following the first administration of the carrier cells. In an example, the method is for increasing the dry weight of a leaf and/or fruit of the plant, such as a tomato plant.

In an example, the weight is wet weight. For example, the method is for increasing plant wet weight (eg, within 1 or 2 weeks of said administration). Optionally, the increase is an increase of at least 5, 10, 11, 12, 13, 14, 15, 16, 17, 18, 19 or 20% compared to a control plant of the same species or strain to which the administration if carrier cells has not taken place, wherein all plants are kept under the same environmental conditions. For example, such an increase is within 1, 2, 3, 4, 5, 6, or 8 weeks following the first administration of the carrier cells. In an example, the method is for increasing the dry weight of a leaf and/or fruit of the plant, such as a tomato plant.

For example, the microbiota is comprised by a leaf, trunk, root or stem of the plant.

The target bacteria (or target cell) may be comprised by a microbiota of a plant. In an example, the microbiota is comprised by a leaf. In an example, the microbiota is comprised by a xylem. In an example, the microbiota is comprised by a phloem. In an example, the microbiota is comprised by a root. In an example, the microbiota is comprised by a tuber. In an example, the microbiota is comprised by a bulb. In an example, the microbiota is comprised by a seed. In an example, the microbiota is comprised by an exocarp, epicarp, mesocarp or endocarp. In an example, the microbiota is comprised by a fruit, eg, a simple fruits; aggregate fruits; or multiple fruits. In an example, the microbiota is comprised by a seed or embryo, eg, by a seed coat; a seed leaf; cotyledons; or a radicle. In an example, the microbiota is comprised by a flower, eg, comprised by a peduncle; sepal: petals; stamen; filament; anther or pistil. In an example, the microbiota is comprised by a root; eg, a tap root system, or a fibrous root system. In an example, the microbiota is comprised by a leaf or leaves, eg, comprised by a leaf blade, petiole or stipule. In an example, the microbiota is comprised by a stem, eg, comprised by bark, epidermis, phloem, cambium. xylem or pith.

For example, the biofilm is comprised by a lung of the subject, eg, wherein the target cells are Pseudomonas (eg, P aeruginosa) cells. This may be useful wherein the subject is a human suffering from a lung disease or condition, such as pneumonia or cystic fibrosis, wherein P1 is a therapeutic protein that is expressed by modified target cells of the biofilm. For example, the biofilm is comprised by an animal or human organ disclosed herein. For example, the biofilm is comprised by a microbiota of a human or animal disclosed herein.

Optionally, said surface is a surface ex vivo, such as a surface comprised by a domestic or industrial apparatus or container.

Optionally, the target cells are comprised by a biofilm, eg, a biofilm as disclosed herein.

There is provided:—

A pharmaceutical composition, livestock growth promoting composition, soil improver, herbicide, plant fertilizer, food or food ingredient sterilizing composition, dental composition, personal hygiene composition or disinfectant composition (eg, for domestic or industrial use) comprising a plurality of the carrier cells.

Herein, a carrier cell is, eg, a commensal or probiotic cell for administration to a human or animal subject. For example, the carrier cell is commensal in a microbiota (eg, gut or blood microbiota) of a human or animal subject, wherein the carrier is for administration to the subject. In an example, a carrier cell is a prokaryotic cell. In an example, a carrier cell is a bacterial cell (and optionally the target cell is a bacterial cell). In an example, a carrier cell is an archaeal cell (and optionally the target cell is an archaeal cell)

Optionally, the carrier cell is a gram-positive bacterial cell and the target cell is a gram-positive bacterial cell.

Optionally, the carrier cell is a gram-positive bacterial cell and the target cell is a gram-negative bacterial cell.

Optionally, the carrier cell is a gram-negative bacterial cell and the target cell is a gram-positive bacterial cell.

Optionally, the carrier cell is a gram-negative bacterial cell and the target cell is a gram-negative bacterial cell.

Optionally, the carrier cell is a Bacteroides bacterial cell and the target cell is a gram-positive bacterial cell. Optionally, the carrier cell is a Bacteroides bacterial cell and the target cell is a gram-negative bacterial cell. Optionally, the carrier cell is a Bacteroides bacterial cell and the target cell is a Bacteroides bacterial cell. Optionally, the carrier cell is a Bacteroides bacterial cell and the target cell is an E coli bacterial cell. Optionally, the carrier cell is an E coli bacterial cell and the target cell is a Bacteroides bacterial cell. For example, in these options the subject is a human or animal, preferably a human.

Optionally, the carrier cell is a Clostridiales bacterial cell and the target cell is a gram-positive bacterial cell. Optionally, the carrier cell is a Clostridiales bacterial cell and the target cell is a gram-negative bacterial cell. Optionally, the carrier cell is a Bacteroides bacterial cell and the target cell is a Clostridiales bacterial cell. Optionally, the carrier cell is a Clostridiales bacterial cell and the target cell is an E coli bacterial cell. Optionally. the carrier cell is an E coli bacterial cell and the target cell is a Clostridiales bacterial cell. For example, in these options the subject is a human or animal, preferably a human.

Optionally, the carrier cell is an Escherichia (eg, E coli) bacterial cell and the target cell is a gram-positive bacterial cell. Optionally, the carrier cell is an Escherichia (eg, E coli) bacterial cell and the target cell is a gram-negative bacterial cell. Optionally, the carrier cell is a Bacteroides bacterial cell and the target cell is an Escherichia (eg, E coli) bacterial cell. Optionally. the carrier cell is an Escherichia (eg, E coli) bacterial cell and the target cell is an E coli bacterial cell. Optionally, the carrier cell is an E coli bacterial cell and the target cell is an Escherichia (eg, E coli) bacterial cell. For example, in these options the subject is a human or animal, preferably a human.

Optionally, the carrier cell is a Bacillus bacterial cell and the target cell is a gram-positive bacterial cell. Optionally, the carrier cell is a Bacillus bacterial cell and the target cell is a gram-negative bacterial cell. Optionally, the carrier cell is a Bacillus bacterial cell and the target cell is a Salmonella bacterial cell. Optionally, the carrier cell is a Bacillus bacterial cell and the target cell is an E coli bacterial cell. Optionally, the carrier cell is an E coli bacterial cell and the target cell is a Pseudomonas bacterial cell. For example, in these options the subject is a plant.

Optionally, the carrier cell is an E coli bacterial cell and the target cell is a gram-positive bacterial cell.

Optionally, the carrier cell is an E coli bacterial cell and the target cell is a gram-negative bacterial cell.

Optionally, the carrier cell is an E coli bacterial cell and the target cell is a Salmonella bacterial cell.

Optionally, the carrier cell is an E coli bacterial cell and the target cell is an E coli bacterial cell.

Optionally, the carrier cell is an E coli bacterial cell and the target cell is a Pseudomonas bacterial cell.

A Bacillus cell herein is optionally a B subtilis cell.

Optionally, the carrier cell is a probiotic or commensal Bacteroides bacterial cell for administration to a human or animal subject. Optionally. the carrier cell is a probiotic or commensal Clostridiales bacterial cell for administration to a human or animal subject. Optionally, the carrier cell is a probiotic or commensal E coli bacterial cell for administration to a human or animal subject. Optionally, the carrier cell is a probiotic or commensal Bacillus bacterial cell for administration to a human or animal subject.

Herein, optionally the plasmid is a closed circular DNA.

In an embodiment, the vector (eg, plasmid) nucleic acid is DNA. Optionally, the DNA is dsDNA. In an embodiment, the vector DNA is ssDNA. In an embodiment, the vector (eg, plasmid) nucleic acid is RNA.

In an example, the target cell is a cell of a species that does not cause nosocomial infection in humans.

Optionally, the target cell is comprised by an animal (eg, poultry animal (such as chicken), swine, cow, fish (eg, catfish or salmon) or shellfish (eg, prawn or lobster)) microbiota. Optionally, the microbiota is a gut microbiota. For example, the target cell is a cell comprised by a human or animal (eg, chicken) gut biofilm. For example, the target cell is a cell comprised by a gut biofilm sample ex vivo. For example, the target cell is a cell comprised by a human or animal (eg, chicken) lung biofilm. For example, the target cell is a cell comprised by a lung biofilm sample ex vivo. For example, the target cell is a cell comprised by a human or animal (eg, chicken) skin biofilm. For example, the target cell is a cell comprised by a skin biofilm sample ex vivo.

In an embodiment, each plasmid comprises an oriV and/or an oriT. In an embodiment, each plasmid comprises a bacterial oriV and/or an oriT.

In an embodiment, the plasmid comprises an oriV and does not encode any replication protein (eg, pir or trfA) that is operable with the oriV to initiate replication of the plasmid.

In an example, the invention relates to a composition comprising a plurality of carrier cells of the invention. Optionally, all of the carrier cells comprise identical said vectors (eg, plasmids). Optionally, the plurality comprises a first sub-population of carrier cells (first cells) and a second sub-population of carrier cells (second cells) wherein the first cells comprise identical first said vectors and the second cells comprise identical second said vectors (which are different from the first vectors of the first cells). For example, the first vectors encode a first guide RNA or crRNA and the second vectors encode a second guide RNA or crRNA, wherein the first guide RNA/crRNA is capable of hybridizing to a first protospacer sequence comprised by the vector in first target cells; and the second guide RNA/crRNA is capable of hybridizing to a second protospacer sequence in carrier or target cells, wherein the protospacers are different.

Optionally, the composition is comprised by a liquid (eg, an aqueous liquid or in water), the composition comprising the carrier cells at an amount of from 1×10³ to 1×10¹⁰ (eg, from 1×10⁴ to 1×10¹⁰; from 1×10⁴ to 1×10⁹; from 1×10⁴ to 1×10⁸; from 1×10⁴ to 1×10⁷; from 1×10⁴ to 1×10¹⁰; from 1×10³ to 1×10⁹; from 1×10³ to 1×10⁸; from 1×10³ to 1×10⁷; from 1×10⁵ to 1×10¹⁰; from 1×10⁵ to 1×10⁹; from 1×10⁵ to 1×10⁸; from 1×10⁵ to 1×10⁷; from 1×10⁶ to 1×10¹⁰; from 1×10⁶ to 1×10⁹; from 1×10⁶ to 1×10⁸; or from 1×10⁶ to 1×10⁷) cfu/ml. For example, the liquid is a beverage, such for human or animal consumption. For example, the beverage is a livestock 30 beverage, eg, a poultry beverage (ie, a beverage for consumption by poultry, such as chicken).

In an example, the composition is a dietary (eg, dietary supplement) composition for consumption by humans or animals. In an example, the composition is a slimming composition for consumption by humans or animals. In an example, the composition is a growth promotion composition for consumption by humans or animals. In an example, the composition is a body building composition for consumption by humans. In an example, the composition is a probiotic composition for consumption by humans or animals. In an example, the composition is a biocidal composition for consumption by humans or animals. In an example, the composition is a pesticidal composition for consumption by humans or animals. In an example, the composition is a zoonosis control composition for consumption by animals.

In an example, the composition comprises vitamins in addition to the carrier cells. In an example, the composition comprises vitamin A, B (eg, B12), C, D, E and/or K in addition to the carrier cells. In an example, the composition comprises lipids in addition to the carrier cells. In an example, the composition comprises carbohydrates in addition to the carrier cells. In an example, the composition comprises proteins and/or amino acids in addition to the carrier cells. In an example, the composition comprises minerals in addition to the carrier cells. In an example, the composition comprises metal ions (eg, Mg²⁺, Cu²⁺ and/or Zn²⁺) in addition to the carrier cells. In an example, the composition comprises sodium ions, potassium ions, magnesium ions, calcium ions, manganese ions, iron ions, cobalt ions, copper ions, zinc ions and/or molybdenum ions.

In an example, the composition is a plant fertilizer composition. In an example, the composition is a herbicide. In an example, the composition is a pesticide composition for application to plants.

In any embodiment or example, where appropriate: The plants are, for example, crop plants. The plants are, for example, wheat. The plants are, for example, corn. The plants are, for example, maize. The plants are, for example, fruiting plants. The plants are, for example, vegetable plants. The plants are, for example, tomato plants. The plants are, for example, potato plants. The plants are, for example, grass plants. The plants are, for example, flowering plants. The plants are, for example, trees. The plants are, for example, shrubs.

In an example, the composition is for environmental application, wherein the environment is an outdoors environment (eg, application to a field or waterway or reservoir). In an example, the composition is comprised by a food or food ingredient (eg, for human or animal consumption). In an example, the composition is comprised by a beverage or beverage ingredient (eg, for human or animal consumption).

In an example the target cell(s) are biofilm cells found in a human, eg, wherein the biofilm is a gut, skin, lung, eye, nose, ear, gastrointestinal tract (GI tract), stomach, hair, kidney, urethra, bronchiole, oral cavity, mouth, liver, heart, anus, rectum, bladder, bowel, intestine, penis, vagina or scrotum biofilm. In an example the target cell(s) are animal biofilm cells, eg, wherein the biofilm is a gut, skin, lung, eye, nose, ear, gastrointestinal tract (GI tract), caecum, jejunum, ileum, colon, stomach, hair, feather, scales, kidney, urethra, bronchiole, oral cavity, mouth, liver, spleen, heart, anus, rectum, bladder, bowel, intestine, penis, vagina or scrotum biofilm. For example, the biofilm is a bird (eg, chicken) caecum biofilm. For example, the biofilm is a bird (eg, chicken) gastrointestinal tract (GI tract), caecum, jejunum, ileum. colon or stomach biofilm.

In an example, any method herein is ex vivo. In an example, a method herein is in vivo. In an example, a method herein is in vitro. In an example, a method herein is carried out in an environment, eg, in a domestic (such as in a house), industrial (such as in a factory) or agricultural environment (such as in a field). In an example, a method herein is carried out in or on a container; or on a surface.

In an example each vector (eg, plasmid) encodes one or more components of a CRTSPR/Cas system operable to perform vector protospacer cutting in the target cell (eg, wherein the protospacer comprises 10-20, 10-30, 10-40, 10-100, 12-15 or 12-20 consecutive nucleotides that are capable of hybridizing in the target cell with a crRNA or gRNA encoded by the vector). For example, the system is a Type I, II, III, IV or V CRISPR/Cas system.

In an example, each vector encodes a Cas9 (and optionally a second, different, Cas, such as a Cas3, Cas9, Cpf1, Cas13a, Cas13b or Cas10); and/or a Cas3 (and optionally a second, different. Cas, such as a Cas3, Cas9, Cpf1, Cas13a, Cas13b or Cas10). In an example, each vector encodes a Cas selected from a Cas3, Cas9, Cpf1, Cas13a, Cas13b and Cas10. Additionally or alternatively, the vector encodes a guide RNA or crRNA or tracrRNA. For example, the guide RNA or crRNA or tracrRNA is cognate to (ie, operable with in the target cell) the first Cas.

In an example, a Cas herein is a Cas9. In an example, a Cas herein is a Cas3. The Cas may be identical to a Cas encoded by the target bacteria.

In an embodiment, each plasmid is a shuttle vector.

Optionally, the target cell is devoid of a functional endogenous CRISPR/Cas system before transfer therein of the vector, eg, wherein the vector encodes a component of an exogenous CRISPR/Cas system that is functional in the target cell. An embodiment provides a medicament comprising a plurality of carrier cells of the invention, wherein each target cell is optionally according to this paragraph, for administration to a human or animal subject for medical use.

In an example, the composition of the invention is a herbicide, pesticide, insecticide, plant fertilizer or cleaning agent.

Optionally, target bacteria herein are comprised by a microbiota of the subject, eg, a gut microbiota. Alternatively, the microbiota is a skin, scalp, hair, eye, ear, oral, throat, lung, blood, rectal, anal, vaginal, scrotal, penile. nasal or tongue microbiota.

In an example the subject (eg, human or animal) is further administered a medicament simultaneously or sequentially with the carrier cell administration. In an example, the medicament is an antibiotic, antibody or antibody fragment (eg, an scFv, nanobody or Fab), immune checkpoint inhibitor (eg, an anti-PD-1, anti-PD-L1 or anti-CTLA4 antibody), adoptive cell therapy (eg. CAR-T therapy) or a vaccine.

In an embodiment, the vector encodes a guided nuclease, such as a Cas nuclease, TALEN, zinc finger nuclease or meganuclease. Thus, P2 may comprise a guided nuclease, such as a Cas nuclease, TALEN, zinc finger nuclease or meganuclease. Optionally, the vector encodes a restriction nuclease that is capable of cutting the vector and/or genome of the carrier cell (eg, chromosome of the carrier cell).

Optionally, the composition is a pharmaceutical composition for use in medicine practised on a human or animal subject. Optionally, the composition is not a medicament.

In an example, the animal is a livestock or companion pet animal (eg, a cow, pig, goat, sheep, horse. dog, cat or rabbit). In an example, the animal is an insect (an insect at any stage of its lifecycle, eg, egg, larva or pupa). In an example, the animal is a protozoan. In an example, the animal is a cephalopod.

Optionally, the composition is a herbicide, pesticide, food or beverage processing agent, food or beverage additive, petrochemical or fuel processing agent, water purifying agent, cosmetic additive, detergent additive or environmental (eg, soil) additive or cleaning agent.

For example, the carrier bacteria are Lactobacillus (eg, L reuteri or L lactis), E coli, Bacillus or Streptococcus (eg, S thermophilus) bacteria. Usefully, the carrier can provide protection for the plasmid from the surrounding environment. The use of a carrier may be useful for oral administration or other routes where the carrier can provide protection for the vector from the acid stomach or other harsh environments in the subject. Furthermore, the carrier can be formulated into a beverage, for example, a probiotic drink, eg, an adapted Yakult (trademark), Actimel (trademark), Kevita (trademark), Activia (trademark), Jarrow (trademark) or similar drink for human consumption.

Optionally, the carrier cell(s) or composition are for administration to a human or animal subject for medical use, comprising killing target bacteria using P1 or a metabolite thereof that is produced in the target cell, wherein the target bacteria mediate as disease or condition in the subject. In an example, when the subject is a human, the subject is not an embryo. In an example, the carrier cells are probiotic in the subject.

Optionally, the environment is a microbiota of soil; a plant, part of a part (e.g., a leaf, fruit, vegetable or flower) or plant product (e.g., pulp); water: a waterway; a fluid; a foodstuff or ingredient thereof; a beverage or ingredient thereof; a medical device; a cosmetic; a detergent; blood; a bodily fluid; a medical apparatus; an industrial apparatus; an oil rig; a petrochemical processing, storage or transport apparatus; a vehicle or a container.

Optionally, the environment is an ex vivo bodily fluid (e.g., urine, blood, blood product, sweat, tears, sputum or spit), bodily solid (e.g., faeces) or tissue of a human or animal subject that has been administered the composition.

Optionally, the environment is an in vivo bodily fluid (e.g., urine, blood, blood product, sweat, tears, sputum or spit), bodily solid (e.g., faeces) or tissue of a human or animal subject that has been administered the composition.

In an embodiment, the plasmid is a phagemid or cloning vector (eg, a shuttle vector, eg, a pUC vector). In an embodiment, the plasmid is a conjugative plasmid.

Optionally, P2 comprises one or more components of a CRISPR/Cas system, eg, a DNA sequence encoding one or more components of Type I Cascade (eg, CasA).

Optionally, P2 comprises a DNA sequence encoding guided nuclease, such as a Cas nuclease. TALEN, zinc finger nuclease or meganuclease.

In an example, the carrier cell(s) or composition are comprised by a medical container, eg, a syringe, vial, TV bag, inhaler, eye dropper or nebulizer. In an example, the carrier cell(s) or composition are comprised by a sterile container. In an example, the carrier cell(s) or composition are comprised by a medically-compatible container. In an example, the carrier cell(s) or composition are comprised by a fermentation vessel, eg, a metal, glass or plastic vessel. In an example, the carrier cell(s) or composition are comprised by an agricultural apparatus. In an example, the carrier cell(s) or composition are comprised by food production or processing apparatus. In an example, the carrier cell(s) or composition are comprised by a horticultural apparatus. In an example, the carrier cell(s) or composition are comprised by a farming apparatus. In an example, the carrier cell(s) or composition are comprised by petrochemicals recovery or processing apparatus. In an example, the carrier cell(s) or composition are comprised by a distillation apparatus. In an example, the carrier cell(s) or composition are comprised by cell culture vessel (eg, having a capacity of at least 50, 100, 1000, 10000 or 100000 litres). Additionally or alternatively, the target cell(s) are comprised by any of these apparatus etc.

In an example, the carrier cell(s) or composition are comprised by a medicament, e,g in combination with instructions or a packaging label with directions to administer the medicament by oral, IV, subcutaneous, intranasal, intraocular, vaginal, topical, rectal or inhaled administration to a human or animal subject. In an example, the carrier cell(s) or composition are comprised by an oral medicament formulation. In an example, the carrier cell(s) or composition are comprised by an intranasal or ocular medicament formulation. In an example, the carrier cell(s) or composition are comprised by a personal hygiene composition (eg, shampoo, soap or deodorant) or cosmetic formulation. In an example, the the carrier cell(s) or composition are comprised by a detergent formulation. In an example, the carrier cell(s) or composition are comprised by a cleaning formulation, eg, for cleaning a medical or industrial device or apparatus. In an example, the carrier cell(s) or composition are comprised by foodstuff, foodstuff ingredient or foodstuff processing agent. In an example, the carrier cell(s) or composition are comprised by beverage, beverage ingredient or beverage processing agent. In an example, the carrier cell(s) or composition are comprised by a medical bandage, fabric, plaster or swab. In an example, the carrier cell(s) or composition are comprised by a herbicide or pesticide. In an example, the carrier cell(s) or composition are comprised by an insecticide.

In an example, the CRISPR/Cas component(s) are component(s) of a Type I CRISPR/Cas system. In an example, the CRISPR/Cas component(s) are component(s) of a Type II CRISPR/Cas system. In an example, the CRISPR/Cas component(s) are component(s) of a Type III CRISPR/Cas system. In an example, the CRISPR/Cas component(s) are component(s) of a Type IV CRISPR/Cas system. In an example, the CRISPR/Cas component(s) are component(s) of a Type V CRISPR/Cas system. In an example, the CRISPR/Cas component(s) comprise a Cas9-encoding nucleotide sequence (eg, S pyogenes Cas9, S aureus Cas9 or S thermophilus Cas9). In an example, the CRISPR/Cas component(s) comprise a Cas3-encoding nucleotide sequence (eg, E coli Cas3, C dificile Cas3 or Salmonella Cas3). In an example, the CRISPR/Cas component(s) comprise a Cpf-encoding nucleotide sequence. In an example, the CRISPR/Cas component(s) comprise a CasX-encoding nucleotide sequence. In an example, the CRISPR/Cas component(s) comprise a CasY-encoding nucleotide sequence.

Optionally, target bacteria are gram negative bacteria (eg, a spirilla or vibrio). Optionally, target bacteria are gram positive bacteria. Optionally, target bacteria are mycoplasma, chlamydiae, spirochete or mycobacterium bacteria. Optionally, target bacteria are Streptococcus (eg, pyogenes or thermophilus). Optionally, target bacteria are Staphylococcus (eg, aureus, eg, MRSA). Optionally, target bacteria are E. coli (eg, O157: H7), eg, wherein the Cas is encoded by the vecor or an endogenous target cell Cas nuclease (eg, Cas3) activity is de-repressed. Optionally, target bacteria are Pseudomonas (eg, syringae or aeruginosa). Optionally, target bacteria are Vibro (eg, cholerae (eg, O139) or vulnificus). Optionally, target bacteria are Neisseria (eg, gonnorrhoeae or meningitidis). Optionally, target bacteria are Borderella (eg, pertussis). Optionally, target bacteria are Haemophilus 10 (eg, influenzae). Optionally, target bacteria are Shigella (eg, dysenteriae). Optionally, target bacteria are Brucella (eg, abortus). Optionally, target bacteria are Francisella host. Optionally, target bacteria are Xanthomonas. Optionally, target bacteria are Agrobacterium. Optionally, target bacteria are Erwinia. Optionally, target bacteria are Legionella (eg, pneumophila). Optionally, target bacteria are Listeria (eg, monocytogenes). Optionally, target bacteria are Campylobacter (eg, jejuni). Optionally, target bacteria are Yersinia (eg, pestis). Optionally, target bacteria are Borelia (eg, burgdorferi). Optionally, target bacteria are Helicobacter (eg, pylori). Optionally, target bacteria are Clostridium (eg, dificile or botulinum). Optionally, target bacteria are Erlichia (eg, chaffeensis). Optionally, target bacteria are Salmonella (eg, typhi or enterica, eg, serotype typhimurium, eg, DT 104). Optionally, target bacteria are Chlamydia (eg, pneumoniae). Optionally, target bacteria are Parachlanmydia host. Optionally, target bacteria are Corynebacterium (eg, amycolatum). Optionally, target bacteria are Klebsiella (eg, pneumoniae). Optionally, target bacteria are Enterococcus (eg, faecalis or faecim, eg, linezolid-resistant). Optionally, target bacteria are Acinetobacter (eg, baumannii, eg, multiple drug resistant).

Further examples of target cells are as follows:—
(a) Optionally the target bacteria are Staphylococcus aureus cells, eg, resistant to an antibiotic selected from methicillin, vancomycin, linezolid, daptomycin, quinupristin, dalfopristin and teicoplanin.
(b) Optionally the target bacteria are Pseudomonas aeruginosa cells, eg, resistant to an antibiotic selected from cephalosporins (eg, ceftazidime), carbapenems (eg, imipenem or meropenem), fluoroquinolones, aminoglycosides (eg, gentamicin or tobramycin) and colistin. (c) Optionally the target bacteria are Klebsiella (eg, pneumoniae) cells, eg, resistant to carbapenem.
(d) Optionally the target bacteria are Streptococcus (eg, thermophilus, pneumoniae or pyogenes) cells, eg, resistant to an antibiotic selected from erythromycin, clindamycin, beta-lactam, macrolide, amoxicillin, azithromycin and penicillin.
(e) Optionally the target bacteria are Salmonella (eg, serotype Typhi) cells, eg, resistant to an antibiotic selected from ceftriaxone, azithromycin and ciprofloxacin.
(f) Optionally the target bacteria are Shigella cells, eg, resistant to an antibiotic selected from ciprofloxacin and azithromycin.
(g) Optionally the target bacteria are Mycobacterium tuberculosis cells, eg, resistant to an antibiotic selected from Resistance to isoniazid (INH), rifampicin (RMP), fluoroquinolone, amikacin, kanamycin and capreomycin and azithromycin.
(h) Optionally the target bacteria are Enterococcus cells. eg, resistant to vancomycin. (i) Optionally the target bacteria are Enterobacteriaceae cells, eg, resistant to an antibiotic selected from a cephalosporin and carbapenem.
(j) Optionally the target bacteria are E. coli cells, eg, resistant to an antibiotic selected from trimethoprim, itrofurantoin, cefalexin and amoxicillin.
(k) Optionally the target bacteria are Clostridium (eg, dificile) cells, eg, resistant to an antibiotic selected from fluoroquinolone antibiotic and carbapenem.
(l) Optionally the target bacteria are Neisseria gonnorrhoea cells, eg, resistant to an antibiotic selected from cefixime (eg, an oral cephalosporin), ceftriaxone (an injectable cephalosporin), azithromycin and tetracycline.
(m) Optionally the target bacteria are Acinetoebacter baumannii cells, eg, resistant to an antibiotic selected from beta-lactam, meropenem and a carbapenem.
(n) Optionally the target bacteria are Campylobacter (eg, jejuni) cells, eg, resistant to an antibiotic selected from ciprofloxacin and azithromycin.
(o) Optionally, the target cell(s) produce Beta (p)-lactamase (eg, ESBL-producing E. coli or ESBL-producing Klebsiella).
(p) Optionally, the target cell(s) are bacterial cells that are resistant to an antibiotic recited in any one of examples (a) to (n).

In an example, the target cell(s) is a cell of a species selected from Shigella, E coli. Salmonella. Serratia, Klebsiella. Yersinia, Pseudomonas and Enterobacter, eg, wherein the subject is a plant. Optionally, the composition comprises carrier cells that are each or in combination capable of conjugative transfer of the vector nucleic acid into target cells of species selected from two or more of Shigella, E coli, Salmonella, Serratia, Klebsiella, Yersinia, Pseudomonas and Enterobacter, eg, wherein the subject is a plant.

In an example, the reduction in growth or proliferation of target cells is at least 50, 60, 70, 80, 90 or 95%.

In embodiments, the plasmid contains a screenable or selectable marker gene. For example, the selectable marker gene is an antibiotic resistance gene.

The carrier bacteria can be bacteria of a species or genus as follows. For example, the species is found in warm-blooded animals (eg, livestock vertebrates). For example, the species is found in humans. For example, the species is found in plants. Preferably. non-pathogenic bacteria that colonize the non-sterile parts of the human or animal body (e.g., skin, digestive tract, urogenital region, mouth. nasal passages, throat and upper airway, ears and eyes) are utilized as carrier cells, and in an example the methodology of the invention is used to combat a target cell bacterial infection of such a part of the body of a human or animal. In another embodiment, the infection is systemic infection. Examples of carrier bacterial species include, but are not limited to: non-pathogenic strains of Escherichia coli (E. coli F18, S17 and E. coli. strain Nissle), various species of Lactobacillus (such as L casei, L plantarum, L paracasei, L. acidophilus. L fermentum, L. zeae and L. gasseri), or other nonpathogenic or probiotic skin- or GI colonizing bacteria such as Lactococcus, Bifidobacteria, Eubacteria, and bacterial mini-cells, which are a nucleoid cells destined to die but still capable of transferring plasmids (see: e.g., Adler et al., Proc. Natl. Acad. Sci. USA 57; 321-326, 1970: Frazer and Curtiss III, Current Topics in Microbiology and Immunology 69: 1-84, 1975; U.S. Pat. No. 4,968,619 to Curtiss III). In some embodiments, the target recipient cells are pathogenic bacteria comprised by a human, animal or plant, eg, on the skin or in the digestive tract, urogenital region, mouth, nasal passage, throat and upper airway, eye(s) and ear(s). Of particular interest for targeting and eradication are pathogenic strains of Pseudomonas aeruginosa. Escherichia coli, Staphylococcus pneumoniae and other species, Enterobacter spp., Enterococcus spp. and Mycobacterium tuberculosis.

The present invention finds use with a wide array of settings or environments, eg, in therapeutic, agricultural, or other settings, including, but not limited to, those described in U.S. Pat. Nos. 6,271,359, 6,261,842, 6,221,582, 6,153,381, 6,106,854, and 5,627,275. Others are also discussed herein, and still others will be readily apparent to those of skill in the art.

A single carrier bacterial strain might harbor more than one type of such vector (eg, differing in the P1 that they encode). Further, in another example two or more different carrier bacterial strains, each containing one or more such vectors. may be combined for producing a plurality of different P1 products in the subject.

The present invention finds utility for treatment of humans and in a variety of veterinary, agronomic, horticultural and food processing applications. For human and veterinary use, and depending on the cell population or tissue targeted for protection, the following modes of administration of the carrier bacteria of the invention are contemplated: topical, oral. nasal, ocular, aural, pulmonary (e.g., via an inhaler), ophthalmic, rectal, urogenital, subcutaneous, intraperitoneal and intravenous. The bacteria may be supplied as a pharmaceutical composition, in a delivery vehicle suitable for the mode of administration selected for the patient being treated. The term “patient” or “subject” as used here may refer to humans or animals (animals being particularly useful as models for clinical efficacy of a particular donor strain, for example, or being farmed or livestock animals). Commercially-relevant animals are chicken, turkey, duck, catfish, salmon, cod, herring, lobster, shrimp, prawns, cows, sheep, goats. pigs, goats, geese or rabbits.

For example, to deliver the carrier bacteria to the gastrointestinal tract or to the nasal passages, the preferred mode of administration may be by oral ingestion or nasal aerosol, or by feeding (alone or incorporated into the subject's feed or food and/or beverage, such as drinking water). In this regard, the carrier cells may be comprised by a food of livestock (or farmed or companion animal), eg, the carrier bacteria are comprised by a feed additive for livestock. Alternatively, the additive is a beverage (eg, water) additive for livestock. It should be noted that probiotic bacteria, such as Lactobacillus acidophilus, are sold as gel capsules containing a lyophilized mixture of bacterial cells and a solid support such as mannitol. When the gel capsule is ingested with liquid, the lyophilized cells are re-hydrated and become viable, colonogenic bacteria. Thus, in a similar fashion, carrier bacterial cells of the present invention can be supplied as a powdered. lyophilized preparation in a gel capsule, or in bulk, eg, for sprinkling onto food or beverages. The re-hydrated, viable bacterial cells will then populate and/or colonise sites throughout the upper and/or lower gastrointestinal system, and thereafter come into contact with the target bacteria.

For topical applications, the carrier bacteria may be formulated as an ointment or cream to be spread on the affected skin surface. Ointment or cream formulations are also suitable for rectal or vaginal delivery, along with other standard formulations, such as suppositories. The appropriate formulations for topical, vaginal or rectal administration are well known to medicinal chemists.

The present invention may be of utility for topical or mucosal administrations to treat a variety of bacterial infections or bacterially related undesirable conditions. Some representative examples of these uses include treatment of (1) conjunctivitis, caused by Haemophilus sp., and corneal ulcers, caused by Pseudomonas aeruginosa; (2) otitis externa, caused by Pseudomonas aeruginosa; (3) chronic sinusitis, caused by many Gram-positive cocci and Gram-negative rods, or for general decontamination of bronchii; (4) cystic fibrosis, associated with Pseudomonas aeruginosa; (5) 35 enteritis, caused by Helicobacter pylori (eg, to treat or prevent gastric ulcers), Escherichia coli, Salmonella typhimurium, Campylobacter or Shigella sp.; (6) open wounds, such as surgical or non-surgical, eg, as a prophylactic measure; (7) burns to eliminate Pseudomonas aeruginosa or other Gram-negative pathogens; (8) acne, eg, caused by Propionobacter acnes; (9) nose or skin infection, eg, caused by methicillin resistant Staphylococcus aureus (MSRA); (10) body odor, eg, caused by Gram-positive anaerobic bacteria (i.e., use of carrier cells in deodorants); (11) bacterial vaginosis, eg, associated with Gardnerella vaginalis or other anaerobes; and (12) gingivitis and/or tooth decay caused by various organisms.

In one example, the target cells are E coli cells and the disease or condition to be treated or prevented in a human is a uterine tract infection or a ventilator associated infection, eg, pneumonia. sepsis, septicaemia or HUS.

In other embodiments, the carrier cells of the present invention find application in the treatment of surfaces for the removal or attenuation of unwanted target bacteria or for modification of bacteria on the surfaces, for example use in a method of treating such a surface or an environment comprising target bacteria, wherein the method comprises contacting the surface or environment with carrier bacteria of the invention, allowing transfer of the vector nucleic acid of the invention from the carrier to the target bacteria, and expressing P1 in target cells. For example, surfaces that may be used in invasive treatments such as surgery, catheterization and the like may be treated to prevent infection of a subject by bacterial contaminants on the surface. It is contemplated that the methods and compositions of the present invention may be used to treat numerous surfaces, objects, materials and the like (e.g., medical or first aid equipment, nursery and kitchen equipment and surfaces) to control bacterial contamination thereon.

Pharmaceutical preparations or other compositions comprising the carrier bacteria may be formulated in dosage unit form for ease of administration and uniformity of dosage. Dosage unit form, as used herein, refers to a physically discrete unit of the pharmaceutical preparation appropriate for the patient or plant or environment or surface undergoing treatment. Each dosage should contain a quantity of the carrier bacteria calculated to produce the desired antibacterial effect in association with the selected carrier. Procedures for determining the appropriate dosage unit are well known to those skilled in the art. Dosage units may be proportionately increased or decreased based on the weight of a patient, plant. surface or environment. Appropriate concentrations for achieving eradication of pathogenic target cells (eg, comprised by a tissue of the patient) may be determined by dosage concentration curve calculations, as known in the art.

Other uses for the carrier bacteria of the invention are also contemplated. These include a variety agricultural, horticultural, environmental and food processing applications. For example, in agriculture and horticulture, various plant pathogenic bacteria may be targeted in order to minimize plant disease. One example of a plant pathogen suitable for targeting is Erwinia (eg, E amylovora, the causal agent of fire blight). Similar strategies may be utilized to reduce or prevent wilting of cut flowers. For veterinary or animal farming, the carrier cells of the invention may be incorporated into animal feed (chicken, swine, poultry, goat, sheep, fish, shellfish or cattle feed) to reduce bio-burden or to eliminate certain pathogenic organisms (e.g., Salmonella, such as in chicken, turkey or other poultry). In other embodiments, the invention may be applied on meat or other foods to eliminate unwanted or pathogenic bacteria (e.g., E. coli O157:H7 on meat, or Proteus spp., one cause of “fishy odour” on seafood).

Environmental utilities comprise, for example, engineering carrier bacteria, eg, Bacillus thuringiensis and one of its conjugative plasmids, to deliver and conditionally express an insecticidal agent in addition to or instead of an antibacterial agent (e.g., for the control of mosquitos that disseminate malaria or West Nile virus). In such applications, as well as in the agricultural and horticultural or other applications described above, formulation of the carrier bacteria as solutions, aerosols, or gel capsules are contemplated.

In an example the plasmid (eg, where the microbiota is a human, anima or plant microbiota) is an engineered RK2 plasmid (ie, a RK2 plasmid that has been modified by recombinant DNA technology or a progeny of such a modified plasmid). Plasmid RK2 is a promiscuous plasmid that can replicate in 29 (and probably many more) gram-negative species (Guiney and Lanka, 1989, p 27-54. In C. M. Thomas (ed) Promiscous plasmids in gram-negative bacteria. London, Ltd London United Kingdom.). Plasmid RK2 is a 60-kb self-transmissible plasmid with a complete nucleotide sequence known (Pansegrau et al., 1994. J. Mol. Biol. 239, 623-663). A minimal replicon derived from this large plasmid has been obtained that is devoid of all its genes except for a trfA gene, that encodes plasmid's Rep protein called TrfA, and an origin of vegetative replication oriV For a review of RK2 replication and its control by TrfA protein, see Helinski et al., 1996 (In Escherichia coli and Salmonella Cellular and Molecular Biology, Vol. 2 (ed. F. Neidhardt, et al., 2295-2324, ASM Press, Washington D.C.).

In an example the plasmid (eg, where the microbiota is a human, anima or plant microbiota) is an engineered R6K plasmid (ie, a R6K plasmid that has been modified by recombinant DNA technology or a progeny of such a modified plasmid).

The present invention is optionally for an industrial or domestic use, or is used in a method for such use. For example, it is for or used in agriculture, oil or petroleum industry, food or drink industry, clothing industry, packaging industry, electronics industry, computer industry, environmental industry, chemical industry, aerospace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.

The present invention is optionally for use in an industry or the environment is an industrial environment, wherein the industry is an industry of a field selected from the group consisting of the medical and healthcare; pharmaceutical; human food; animal food; plant fertilizers; beverage; dairy; meat processing; agriculture; livestock farming; poultry farming; fish and shellfish farming; veterinary; oil; gas; petrochemical; water treatment; sewage treatment; packaging; electronics and computer; personal healthcare and toiletries; cosmetics; dental; non-medical dental; ophthalmic; non-medical ophthalmic; mineral mining and processing; metals mining and processing; quarrying; aviation; automotive; rail; shipping; space; environmental; soil treatment; pulp and paper; clothing manufacture; dyes; printing; adhesives; air treatment; solvents; biodefence; vitamin supplements; cold storage; fibre retting and production; biotechnology; chemical; industrial cleaning products; domestic cleaning products; soaps and detergents; consumer products: forestry; fishing; leisure; recycling; plastics; hide, leather and suede; waste management; funeral and undertaking; fuel; building; energy; steel; and tobacco industry fields.

In an example, the plasmid comprises a CRISPR array, wherein the array comprises one, or two or 20 more different spacers (eg, 2, 3, 4, 5, 6, 7, 8, 9, 10, 20, 30, 40, 50 or more spacers) for targeting the vector and/or the genome of a carrier bacterium.

In an example, the target bacteria are comprised by an environment as follows. In an example, the environment is a microbiota of a human, eg, the oral cavity microbiota or gut microbiota or the bloodstream. In an example, the environment is not an environment in or on a human. In an example, the environment is not an environment in or on a non-human animal. In an embodiment, the environment is an air environment. In an embodiment, the environment is an agricultural environment. In an embodiment, the environment is an oil or petroleum recovery environment, eg, an oil or petroleum field or well. In an example, the environment is an environment in or on a foodstuff or beverage for human or non-human animal consumption. In an example, the environment is a maritime environment, eg, in seawater or on a boat (eg, in ship or boat ballast water).

In an example, the environment is a human or animal microbiota (eg, gut, vaginal, scalp, armpit, skin or oral cavity microbiota). In an example, the target bacteria are comprised by a human or animal microbiota (eg, gut, vaginal, scalp, armpit, skin or oral cavity microbiota).

In an example, the carrier bacteria or composition of the invention are administered intranasally, topically or orally to a human or non-human animal, or is for such administration. The skilled person aiming to treat a microbiota of the human or animal will be able to determine the best route of administration, depending upon the microbiota of interest. For example, when the microbiota is a gut microbiota, administration can be intranasally or orally. When the microbiota is a scalp or armpit microbiota, administration can be topically. When the microbiota is in the mouth or throat, the administration can be orally.

In an example, the environment is harboured by a beverage or water (eg, a waterway or drinking water for human consumption) or soil. The water is optionally in a heating. cooling or industrial system, or in a drinking water storage container.

In an example, the carrier and/or target bacteria are Firmicutes selected from Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutvrivibrio. Pseudoramibacter, Roseburia, Ruminococcus, Sarcina, Seinonella, Shuttleworthia, Sporobacter, Sporobacterium, Streptococcus, Subdoligranulum, Syntrophococcus, Thernobacillus, Turibacter and Weisella.

In an example, the carrier bacteria, composition, use or method is for reducing pathogenic infections or for re-balancing gut or oral biofilm eg, for treating or preventing obesity or disease in a human or animal; or for treating or preventing a GI condition (such as Crohn's disease, IBD or colitis). For example, the vector, carrier bacteria, composition, use or method is for knocking-down Salmonella, Campylobacter, Erwinia, Xanthomonas, Edwardsiella, Pseudomonas, Klebsiella, Pectobacterium, Clostridium dificile or E coli bacteria in a gut biofilm of a human or animal or a plant, preferably in a human or animal.

In an example, the animal is a chicken, eg, and the target bacteria are Salmonella or Campylobacter. In an example, the animal is a fish (eg, catfish or salmon) or shellfish (eg, prawn or lobster), eg, and the target bacteria are Edwardsiella. In an example, the plant is a potato plant and, eg, the target bacteria are Pectobacterium. In an example, the plant is a cabbage plant and, eg, the target bacteria are Xanthomonous (eg, X campestris). In an example, the plant is a marijuana plant and, eg, the target bacteria are Pseudomonas (eg, P cannabina or P amygdali), Agrobacterium (eg, A tumefaciens) or Xanthomonas (eg, X campestris). In an example, the plant is a hemp plant and, eg, the target bacteria are Pseudomonas (eg, P cannabina or P amygdali), Agrobacterium (eg, A tumefaciens) or Xanthomonas (eg, X campestris).

Optionally, the environment is comprised by, or the target bacteria are comprised by, a gut biofilm, skin biofilm, oral cavity biofilm, throat biofilm, hair biofilm, armpit biofilm, vaginal biofilm, rectal biofilm, anal biofilm, ocular biofilm, nasal biofilm, tongue biofilm, lung biofilm, liver biofilm, kidney biofilm, genital biofilm, penile biofilm, scrotal biofilm, mammary gland biofilm, ear biofilm, urethra biofilm, labial biofilm, organ biofilm or dental biofilm. Optionally, the environment is comprised by, or the target bacteria are comprised by, a plant (eg, a tobacco, crop plant, fruit plant, vegetable plant or tobacco, eg on the surface of a plant or contained in a plant) or by an environment (eg, soil or water or a waterway or aqueous liquid).

### Diseases & Conditions

In an example, the carrier cell(s) or composition is for treating a disease or condition in an animal or human. In an example, the disease or condition is caused by or mediated by the presence of a protein or metabolite in the human or animal subject and expression of P1 by target cells comprising the vector nucleic acid causes a reduction in the protein or metabolite in the subject. In an example, the disease or condition is caused by or mediated by the absence of a protein or metabolite in the human or animal subject and expression of P1 by target cells comprising the vector nucleic acid causes an increase in the protein or metabolite in the subject. In an example, the disease or condition is caused by an undesirably high level of a protein or metabolite in the human or animal subject and expression of P1 by target cells comprising the vector nucleic acid causes an decrease in the protein or metabolite in the subject. In an example, the disease or condition is caused by an undesirably low level of a protein or metabolite in the human or animal subject and expression of P1 by target cells comprising the vector nucleic acid causes an increase in the protein or metabolite in the subject. The metabolite may be a protein, peptide, amino acid, carbohydrate, sugar, lipid, fatty acid or ion (eg, a metal ion). The metabolite may be toxic to human cells of the human subject. The metabolite may be a hormone, growth factor or antibiotic. The metabolite may be a mineral. The metabolite may be a salt. The metabolite may be a nucleic acid, eg, a RNA (eg, a mRNA) or DNA.

In an example, the disease or condition is a cancer, inflammatory or autoimmune disease or condition, eg, obesity, diabetes IBD, a GI tract condition or an oral cavity condition.

Optionally, the disease or condition of a human or animal subject is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation; (f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis. psoriasis, eczema, asthma, ulcerative colitis, colitis, Crohn's disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovascularisation (eg, of a tumour or in the eye);
(jj) Common cold;
(kk) Epilepsy;
(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jj) A bone disorder;
(kkk) Sepsis or septic shock;
(lll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

### Neurodegenerative or CNS Diseases or Conditions for Treatment or Prevention by the Invention

In an example, the neurodegenerative or CNS disease or condition is selected from the group consisting of Alzheimer disease, geriopsychosis, Down syndrome, Parkinson's disease, Creutzfeldt-jakob disease, diabetic neuropathy, Parkinson syndrome, Huntington's disease. Machado-Joseph disease, amyotrophic lateral sclerosis, diabetic neuropathy, and Creutzfeldt Creutzfeldt-Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and/or reduces the progression of nerve fibre damage. In an example, the method restores nerve myelin and/or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatment and/or prevention of CNS and neurodegenerative diseases and conditions, eg, agents such as immune checkpoint inhibitors, eg, anti-PD-1, anti-PD-L1, anti-TIM3 or other antibodies disclosed therein).

### Cancers for Treatment

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or hematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pineaioma, hemangioblastoma, acoustic neuroma, oligodendroglioma, menangioma, neuroblastoma, retinoblastoma and brain metastases).

### Autoimmune Disease for Treatment or Prevention

1. Acute Disseminated Encephalomyelitis (ADEM)
2. Acute necrotizing hemorrhagic leukoencephalitis
3. Addison's disease
4. Agammaglobulinemia
5. Alopecia areata
6. Amyloidosis
7. Ankylosing spondylitis
8. Anti-GBM/Anti-TBM nephritis
9. Antiphospholipid syndrome (APS)
10. Autoimmune angioedema
11. Autoimmune aplastic anemia
12. Autoimmune dysautonomia
13. Autoimmune hepatitis
14. Autoimmune hyperlipidemia
15. Autoimmune immunodeficiency
16. Autoimmune inner ear disease (AIED)
17. Autoimmune myocarditis
18. Autoimmune oophoritis
19. Autoimmune pancreatitis
20. Autoimmune retinopathy
21. Autoimmune thrombocytopenic purpura (ATP)
22. Autoimmune thyroid disease
23. Autoimmune urticaria
24. Axonal & neuronal neuropathies
25. Balo disease
26. Behcet's disease
27. Bullous pemphigoid
28. Cardiomyopathy
29. Castleman disease
30. Celiac disease
31. Chagas disease
32. Chronic fatigue syndrome
33. Chronic inflammatory demyelinating polyneuropathy (CIDP)
34. Chronic recurrent multifocal ostomyelitis (CRMO)
35. Churg-Strauss syndrome
36. Cicatricial pemphigoid/benign mucosal pemphigoid
37. Crohn's disease
38. Cogans syndrome
39. Cold agglutinin disease
40. Congenital heart block
41. Coxsackie myocarditis
42. CREST disease
43. Essential mixed cryoglobulinemia
44. Demyelinating neuropathies
45. Dermatitis herpetiformis
46. Dermatomyositis
47. Devic's disease (neuromyelitis optica)
48. Discoid lupus
49. Dressler's syndrome
50. Endometriosis
51. Eosinophilic esophagitis
52. Eosinophilic fasciitis
53. Erythema nodosum
54. Experimental allergic encephalomyelitis
55. Evans syndrome
56. Fibromyalgia
57. Fibrosing alveolitis
58. Giant cell arteritis (temporal arteritis)
59. Giant cell myocarditis
60. Glomerulonephritis
61. Goodpasture's syndrome
62. Granulomatosis with Polyangiitis (GPA) (formerly called Wegener's Granulomatosis)
63. Graves' disease
64. Guillain-Barre syndrome
65. Hashimoto's encephalitis
66. Hashimoto's thyroiditis
67. Hemolytic anemia
68. Henoch-Schonlein purpura
69. Herpes gestationis
70. Hypogammaglobulinemia
71. Idiopathic thrombocytopenic purpura (ITP)
72. IgA nephropathy
73. IgG4-related sclerosing disease
74. Immunoregulatory lipoproteins
75. Inclusion body myositis
76. Interstitial cystitis
77. Juvenile arthritis
78. Juvenile diabetes (Type 1 diabetes)
79. Juvenile myositis
80. Kawasaki syndrome
81. Lambert-Eaton syndrome
82. Leukocytoclastic vasculitis
83. Lichen planus
84. Lichen sclerosus
85. Ligneous conjunctivitis
86. Linear IgA disease (LAD)
87. Lupus (SLE)
88. Lyme disease, chronic
89. Meniere's disease
90. Microscopic polyangiitis
91. Mixed connective tissue disease (MCTD)
92. Mooren's ulcer
93. Mucha-Habermann disease
94. Multiple sclerosis
95. Myasthenia gravis
96. Myositis
97. Narcolepsy
98. Neuromyelitis optica (Devic's)
99. Neutropenia
100. Ocular cicatricial pemphigoid
101. Optic neuritis
102. Palindromic rheumatism
103. PANDAS (Pediatric Autoimmune Neuropsychiatric Disorders Associated with Streptococcus)
104. Paraneoplastic cerebellar degeneration
105. Paroxysmal nocturnal hemoglobinuria (PNH)
106. Parry Romberg syndrome
107. Parsonnage-Turner syndrome
108. Pars planitis (peripheral uveitis)
109. Pemphigus
110. Peripheral neuropathy
11. Perivenous encephalomyelitis
112. Pernicious anemia
113. POEMS syndrome
114. Polyarteritis nodosa
115. Type I, H, & II autoimmune polyglandular syndromes
116. Polymyalgia rheumatica
117. Polymyositis
118. Postmyocardial infarction syndrome
119. Postpericardiotomy syndrome
120. Progesterone dermatitis
121. Primary biliary cirrhosis
122. Primary sclerosing cholangitis
123. Psoriasis
124. Psoriatic arthritis
125. Idiopathic pulmonary fibrosis
126. Pyoderma gangrenosum
127. Pure red cell aplasia
128. Raynauds phenomenon
129. Reactive Arthritis
130. Reflex sympathetic dystrophy
131. Reiter's syndrome
132. Relapsing polychondritis
133. Restless legs syndrome
134. Retroperitoneal fibrosis
135. Rheumatic fever
136. Rheumatoid arthritis
137. Sarcoidosis
138. Schmidt syndrome
139. Scleritis
140. Scleroderma
141. Sjogren's syndrome
142. Sperm & testicular autoimmunity
143. Stiff person syndrome
144. Subacute bacterial endocarditis (SBE)
145. Susac's syndrome
146. Sympathetic ophthalmia
147. Takayasu's arteritis
148. Temporal arteritis/Giant cell arteritis
149. Thrombocytopenic purpura (TTP)
150. Tolosa-Hunt syndrome
151. Transverse myelitis
152. Type I diabetes
153. Ulcerative colitis
154. Undifferentiated connective tissue disease (UCTD)
155. Uveitis
156. Vasculitis
157. Vesiculobullous dermatosis
158. Vitiligo
159. Wegener's granulomatosis (now termed Granulomatosis with Polyangiitis (GPA).

### Inflammatory Diseases for Treatment or Prevention

1. Alzheimer
2. ankylosing SPONDYLITIS
3. arthritis (osteoarthritis, rheumatoid arthritis (RA), psoriatic arthritis)
4. asthma
5. atherosclerosis
6. Crohn's disease
7. colitis
8. dermatitis
9. diverticulitis
10. fibromyalgia
11. hepatitis
12. irritable bowel syndrome (IBS)
13. systemic lupus erythematous (SLE)
14. nephritis
15. Parkinson's disease
16. ulcerative colitis.

For example, the composition comprising carrier cells is a human or animal food and/or beverage (eg, mixed in drinking water for livestock consumption). When supplied in a beverage, the vector may be comprised by carrier bacteria, wherein the carrier bacteria are comprised in the beverage at an amount of from 1×10³ to 1×10¹⁰ (eg, from 1×10⁴ to 1×10¹⁰; from 1×10⁴ to 1×10⁹; from 1×10⁴ to 1×10⁸; from 1×10⁴ to 1×10⁷; from 1×10⁵ to 1×10¹⁰; from 1×10⁵ to 1×10⁹; from 1×10³ to 1×10⁸; from 1×10⁵ to 1×10⁷; from 1×10⁶ to 1×10¹⁰; from 1×10⁵ to 1×10⁹; from 1×10⁵ to 1×10⁸; from 1×10⁵ to 1×10⁷; from 1×10⁶ to 1×10¹⁰; from 1×10⁶ to 1×10⁹; from 1×10⁶ to 1×10⁸; or from 1×10⁶ to 1×10⁷) cfu/ml. When supplied in a beverage, the vector may be comprised by carrier bacteria, wherein the carrier bacteria are comprised in the beverage at an amount of at least 1×10¹ cfu/ml, eg, wherein the subject is a human or animal (eg, a poultry bird, such as a chicken).

Optionally, the guided nuclease is any guided nuclease disclosed herein, eg, a Cas, TALEN, meganuclease or a zinc finger nuclease. In an example, the component is a crRNA or guide RNA that is operable in target cells with a cognate Cas nuclease. The Cas nuclease can be any Cas nuclease disclosed herein. The Cas nuclease may be an endogenous Cas of the target cells or may be encoded by an exogenous nucleic acid that is administered to the animal.

There is provided according to the Fifth Configuration:

A method of engineering a microbiome (eg, any microbiome disclosed herein), the method comprising contacting the microbiome with a plurality of vectors as described herein (eg, by combining the microbiome with carrier cells as described) and optionally allowing transfer of said vector nucleic acid into target cells of the microbiota.

A modified microbiota obtained or obtainable by the method herein, optionally wherein the microbiota is comprised by a pharmaceutical composition for use as a medicament to treat a disease or condition in a human or animal subject.

Any of the features disclosed in the context of the First to Fourth Configurations may apply mutatis mutandis to the Fifth Configuration.

### Concepts

The invention also provides the following Concepts.

1. A host cell comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to regulate expression or activity of P1; wherein
(c) NS1 is under the control of a first promoter (eg, a constitutive or inducible promoter) for expression of P1; and
(d) expression or activity of P2 in the host cell is regulatable by exposure of the host cell comprising the at least one vector to a regulator agent (R), thereby regulating the expression or activity of Pt;
wherein the host cell is a bacterial, archaeal or fungal cell.

Optionally, the nucleic acid is comprised by at least one nucleic acid vector for transfer from the host cell into a cell of a microbiota. For example the host cell and microbiota cell are bacterial cells.

For example, the fungal cell is a yeast cell. The bacterial cell may be a cell of any bacterial species or genus disclosed herein.

2. The cell of Concept 1, wherein
(a) the nucleic acid is comprised by at least one nucleic acid vector of the cell;
(b) NS1 is comprised by a nucleic acid vector of the cell and NS2 is comprised by a chromosome of the cell;
(c) NS1 is comprised by a chromosome of the cell and NS2 is comprised by a nucleic acid vector of the cell; or
(d) NS1 is comprised by a chromosome of the cell and NS2 is comprised by a chromosome of the cell.

3. The cell of any one of Concept 2(a)-(c) wherein the vector or each vector is a conjugative plasmid for transfer to a cell of a microbiota comprised by a human or animal subject.

4. The cell of any preceding Concept, wherein the cell is a cell of commensal or probiotic bacterial species of a human or animal microbiota, optionally an E coli cell or a Bacteroides cell.

5. The cell of any preceding Concept, wherein NS2 is under the control of a second promoter that is regulatable for expression of P2, wherein binding of a regulator agent (R) to the vector nucleic acid regulates the second promoter, thereby regulating the expression of P2 and P1.

6. The cell of any preceding Concept, wherein P2 comprises an RNA-guided nuclease (optionally a Cas nuclease), wherein the nuclease is operable to cut the nucleic acid at a predetermined sequence motif.

7. The cell of Concept 6, wherein
(a) the sequence motif is comprised by a chromosome of the cell and the cutting kills the cell; or wherein the sequence motif is comprised by a gene (on a chromosome or vector of the cell) comprising NS1 for production of P1 and the cutting down-regulates the production of P1; or
(b) the sequence motif is comprised by a said vector comprising NS1.

This is useful for promoting degradation of the vector, thereby reducing or inhibiting the production of P1. For example, the cut vector is degraded in the cell.

8. The cell of any preceding Concept for treating or preventing a disease or condition in a human or animal subject, wherein the cell is administered to a microbiota (optionally a gut microbiota) of the subject to produce P1 in the subject thereby treating or preventing the disease or condition in the subject.

### Regulation by Xylitol or Xylose

Over half of ingested xylitol in humans is not adsorbed by human cells instead reaching the gastrointestinal tract where it is taken up by the microbiome (Livesey, 2003). making this useful to control our switches in the GI tract. As exemplified herein, we advantageously found that xylitol induces expression by lifting the repression that the transcriptional regulator exerts on the promoter. Furthermore, E. coli strains generally lack the xylose reductase and xylitol dehydrogenase typically necessary for xylitol metabolism (Ge et al., 2018). We realised that this may be useful for more precise or lasting control by xylitol when the strain cannot metabolise xylitol. Use of lower doses of xylitol may be possible when the strain cannot metabolise xylitol. Thus, in an example the cell of the invention is an E coli cell and the promoter is regulatable by xylitol.

1. A cell (optionally according to any preceding Concept), comprising a nucleic acid, wherein the nucleic acid comprises a gene encoding a product of interest (P1), the gene comprising a nucleotide sequence (NS1) encoding P1 and a regulatory region 5′ of NS1 that comprises a promoter (Px) for controlling the expression of NS1, wherein the combination of Px and NS1 is heterologous to the cell and Px is regulatable by xylitol or xylose.

For example, NS1 is not found in a wild-type cell of the same species as the cell of the invention. For example, NS1 is a non-bacterial (eg, an animal, human, mammal or plant) sequence. Preferably, NS1 is a human sequence.

For example, NS1 is an endogenous sequence of the cell and Px is heterologous to the cell.

2. The cell of Concept 1, wherein the promoter is a xylitol or xylose regulatable promoter of a Morganella species, optionally M morganii.

For example, the M morganii is Morganella morganii strain ZJG812.

3. The cell of Concept I or 2, wherein the promoter comprises SEQ ID NO: 3 or a nucleotide sequence that is at least 70% identical to SEQ ID NO: 3.

For example, any percent identity herein is at least 70, 80, 90, 95, 96, 97, 98 or 99%.

Optionally, the cell comprises SEQ ID NO: 4 or a nucleotide sequence encoding a xylitol regulatable promoter. For example, the sequence further encodes a repressor that is cognate to the promoter. For example, the sequence further encodes a xylitol isomerase.

4. The cell of any one of Concepts 1-3, wherein Px is homologous to a xylitol or xylose regulatable promoter of Morganella morganii.

For example, the promoter is a xylitol regulatable promoter. For example, the promoter is repressible by a repressor and xylitol is capable of de-repressing the repressor.

5. The cell of any one of Concepts 1-4, wherein the cell genome encodes a repressor that is capable of repressing Px, wherein xylitol and/or xylose is capable of de-repressing the repressor.

6. The cell of Concept 5, wherein the repressor is encoded by SEQ ID NO: I or a nucleotide sequence that is at least 70% identical to SEQ ID NO: 1.

For example, the identity is at least 70, 80, 90, 95, 96, 97, 98 or 99%.

For example, the repressor comprises SEQ ID NO: 2 or an amino acid sequence that is at least 70% identical to SEQ ID NO: 2.

7. The cell of any one of Concepts 1-6, wherein the cell comprises a xylitol transporter, optionally a xylitol ABC transporter.

Our data surprisingly suggest that the presence of a xylitol transporter increases the sensitivity of the donor strains towards xylitol in the growth medium.

8. The cell of any one of Concepts 1-7, wherein the cell is devoid of a xylitol isomerase gene.

For example, the cell genome comprises a xylitol inducible promoter of a Morganii species and excludes a sugar isomerase gene. For example, the cell comprises a xylitol inducible promoter of a Morganii species and excludes a nucleotide sequence encoding a xylitol isomerase.

In an example, the cell genome comprises a xylABC operon from Morganella morganii. Optionally the operon is devoid of a nucleotide sequence encoding a xylitol isomerase.

9. A nucleic acid vector comprising a gene as recited in any one of Concepts 1-8.

10. The vector of Concept 9, wherein the vector is a plasmid (optionally a conjugative plasmid), transposon, phagemid or a phage.

In an example, the cell of the invention comprises any vector of the invention as described herein.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications and all US equivalent patent applications and patents are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps The term “or combinations thereof” or similar as used herein refers to all permutations and combinations of the listed items preceding the term. For example, “A, B, C, or combinations thereof is intended to include at least one of: A, B, C, AB. AC. BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EXAMPLES

### Example 1: Construction of Conjugative and Self-Targeting Production CGV

### Background to Example 1

The pBBR1 based plasmid used contains an oriT that is mobilizable by the RP4 plasmid transfer machinery carried in trans in the E. coli JKE201 strain (see: Harms, A., Liesch, M., Kurner, J., Qudbatte, M., Engel, P. and Dehio, C., 2017. A bacterial toxin-antitoxin module is the origin of inter-bacterial and inter-kingdom effectors of Bartonella. PLOS Genetics, 13(10), p. e1007077) which will harbour the plasmid.
The genes encoding the E. coli Type I-E Cas3 were cloned under the control of a pBad promoter (which is induced by exposure to arabinose). A nucleotide sequence encoding Superfolder Green Fluorescent Protein (sfGFP) and a sfGFP-targeting CRISPR array controlled by a rhamnose inducible promoter will be inserted into the replicon. The array will encode one or more crRNAs for the formation of guide RNAs, that are cognate with the Cas3 and operable to target the sfGFP sequence to guide the Cas3 to the sequence and cut it.

### Plasmid Components:

pBBR1-msc2, a mobilizable shuttle and expression vector;
oriT from the RP4 plasmid;
A Kanamycin antibiotic resistance marker;
rmtB (16S rRNA methylase gene) which gives resistance to amikacin and gentamicin
E coli CRISPR/cas system with pBAD promoter, wherein the system comprises nucleotide sequences encoding Type I-E Cas3, Cascade proteins A-E under the control of a pBAD promoter, the system also comprising the CRISPR array;
S. pyogenes terminator after the Cascade.
rhamnose promoter in front of the CRISPR array, which is programmed to target the sfGFP gene (contains one spacer that can hybridise to the sfGFP sequence); and
constitutively expressed sfGFP gene.

The order of the components:

Kanamycin resistance gene, pBBR1-msc2, oriT from RP4 plasmid, araC. pBad promoter, E. coli Cas3 Type I-E sequence, Cascade sequence, S. pyogenes terminator, rhamnose promoter, array comprising spacer that can hybridise to the sfGFP sequence, rmtB (16S rRNA methylase gene) which gives resistance to amikacin and gentamicin, sfGFP gene

### Methods, Cloning:

### Backbone

p1075 containing the pBBR1 replicon and CRISPR cas system, was used as a template for cloning sfGFP gene, S. pyogenes terminator after the E. coli Type I-E Cas3 and Cascade, CRISPR array with sfGFP target and rhamnose promoter to control CRISPR array with sfGFP target (see plasmid map FIG. 1).

Transformation, selection and sequence verification

Transformation technique: The final construct (p1364) is electroporated to the strain of interest (NEB 10-beta cells for cloning or JKE201 strain for further conjugation). Correct colonies are selected on 3 antibiotic markers (kanamycin, gentamicin, amikacin), colonies will have a distinctive green color (due to expression of GFP) and with confirmation using colony PCR with the construct specific primers.

Sequence verification is performed by subjecting the purified plasmid to sequencing with construct specific primers to cover the whole construct.

See FIG. 1 for a plasmid map highlighting the main components of the final plasmid, p1364.

### Example 2: Conjugative Delivery from E. coli Donor Strain JKE201 to Recipient E. coli Strain b52

### Methods

The JKE201 strain was transformed with p1364 and a positive clone was selected on kanamycin LB plates (supplemented with DAP) and further verified by the positive GFP signal. One colony of the transformed JKE201/p1364 donor strain as well as the b52 (strain C-la-CGSC, obtained from the Coli Genetic Stock Center) recipient strain was grown to exponential phase in liquid LB (JKE201 supplemented with DAP). The two cultures were then mixed 5:1 (JKE201/p1364: b52) and spotted solid LB agar to incubate overnight.

After overnight conjugation, the mix was scraped off and dissolved in liquid LB and plated on LB plates containing kanamycin but without DAP to select only b52 transconjugants. These were enumerated compared to total recipient counts on LB plates.

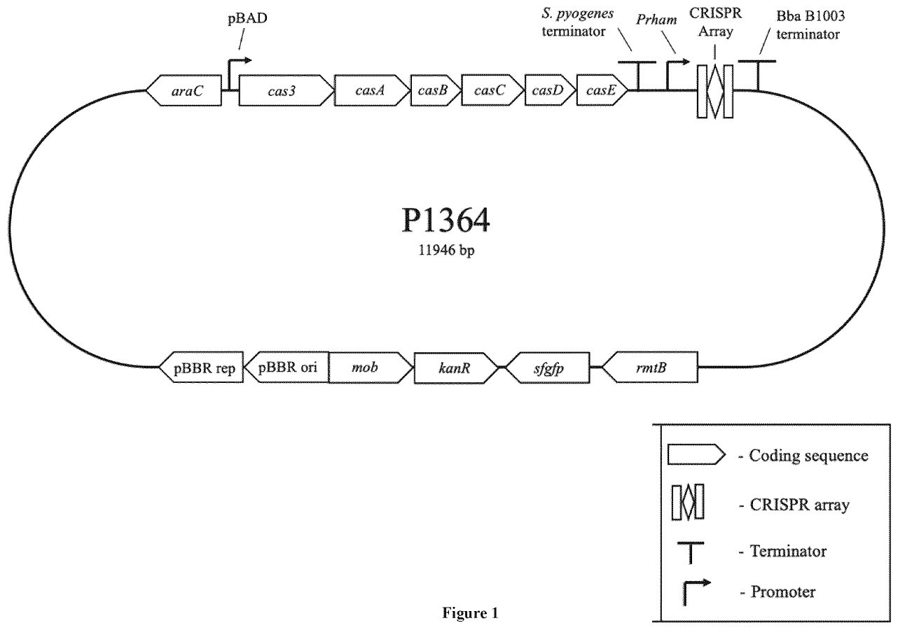

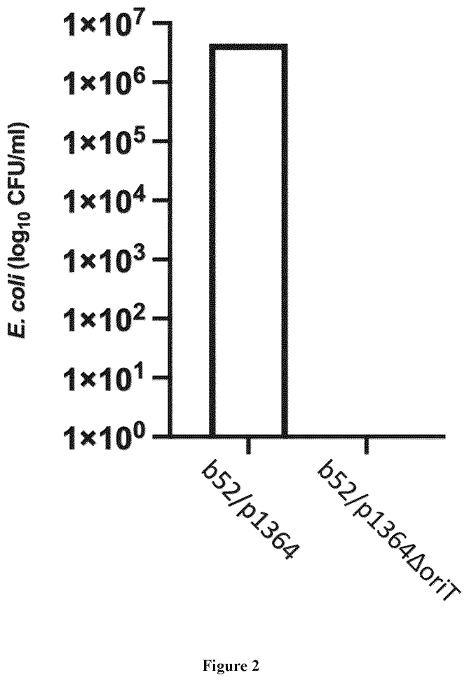

Results are depicted in FIG. 2.

The p1364 transfers at high frequency into the b52 model strain. FIG. 2 shows the number of transconjugants formed as a result of the RP4 oriT on p1364 transfer and the resultant absence of transconjugants when oriT is absent.

### Example 3: Production of Protein of Interest from Mobilizable Plasmid in ‘Production Bacteria’ Strain (Recipient Strain) b52

Transconjugant recipient cells (b52 cells containing plasmid p1364) were produced as described in Example 2. The transconjugants, containing the sfGFP expressing construct with a self-targeting CRISPR array, conjugative plasmid p1364, was grown in LB medium to and OD600 of 1. A plasmid-free b52 strain was grown to the same level for use as a negative/autofluorescence control. Each culture was transferred to a microtiter plate and measured in a Synergy™ H1 plate reader. sfGFP production was quantified using and excitation wavelength of 485 nm and an emission wavelength of 510 nm.

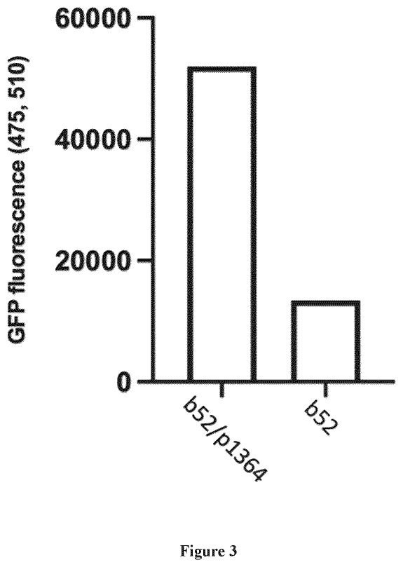

Results are depicted in FIG. 3. GFP fluorescence was determined in in b52 cells harbouring the p1364 plasmid and in b52 control cells devoid of the plasmid at OD=0.7. The GFP fluorescence was approximately five times higher than the background fluorescence of the b52 control strain, showing reliable expression of sfGFP.

### Example 4: Removal of Conjugative Plasmid from Recipient Strain b52

Transconjugants b52 cells (from Example 2) carrying the p1364 plasmid were grown with selection for p1364 (kanamycin) for 2 h and subsequently washed in LB to remove residual antibiotics.

The resuspended culture was then split in two 1 ml portions and inducers (I % arabinose, 10 mM of rhamnose) were added to one of the tubes to induce the self-targeting Cas mechanism. Just after induction, cells from both cultures were plated on LB plates (without inducers added, timepoint 0). Subsequently, samples from each tube (induced and non-induced) were plated on LB plates at timepoints: 1 h and 24 hrs after induction. All plates were incubated overnight after which the proportion of sfGFP expressing cells was determined by colony counting in a blue light transilluminator (365 nm, UV benchtop transilluminator, VWR®).

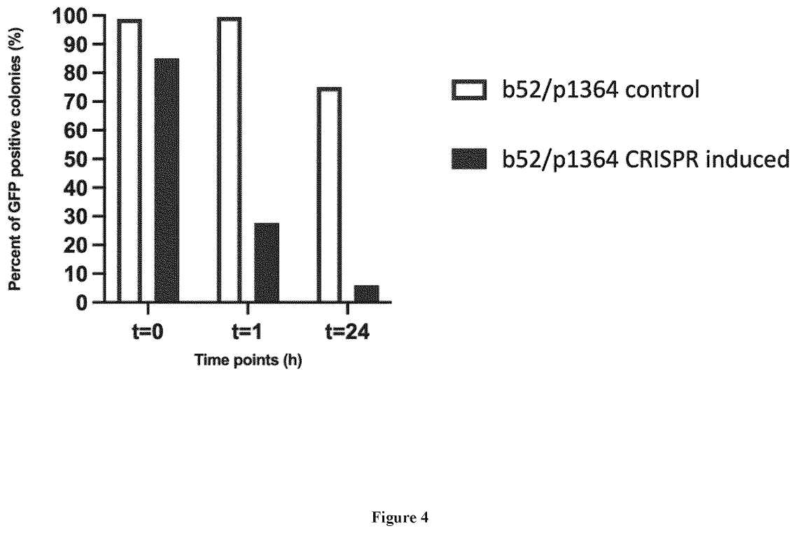

Results are depicted in FIG. 4 and shows the proportion of GFP positive cells over time for induced and non-induced cultures. The induction of the CRIPSR system and corresponding array on p1364 allows for rapid curing of the self-targeting plasmid. The system allows for >70% plasmid curing within 1 h and >95% of the plasmid purged within 24 hrs.

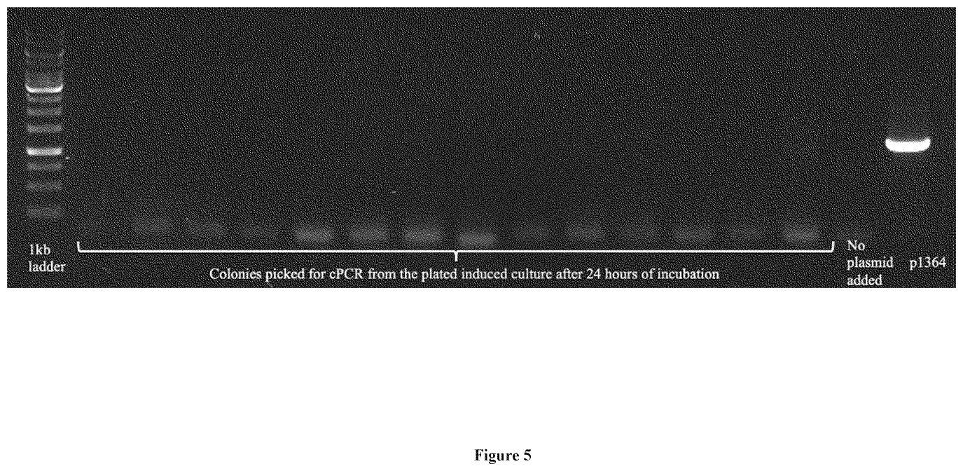

To validate that the loss of sfGFP was indeed due to plasmid loss, PCR specific for the plasmid backbone was performed on 14 randomly selected colonies from the plate of the 24 h timepoint. See the resulting gel image in FIG. 5.

### Example 5: King Donor Strain by CRISPR

A conjugative plasmid construct was made that could be introduced into host cells to produce donor cells. The plasmid will be a vector for a gene of interest (eg, GFP in the examples above) and a self-targeting CRISPR/array to target the plasmid in recipient cells after it has been transferred by conjugation from donor to recipient cells.

The plasmid was engineered so that, on induction of the CRISPR/cas system with arabinose, the plasmid was able to target the genome of the host cells. The plasmid was made using the RP4 plasmid as a starting point. The arabinose inducible E. coli Type I-E Cas3 Cascade followed by a CRISPR array for producing a guide RNA targeting the E. coli chromosomal gene lptA was integrated into the RP4 plasmid using recombineering.

The Cas system and array with the arabinose promoter was amplified along with a spectinomycin resistance gene and araC from a cloning vector containing these.

The PCR product were flanked with homology arms that allowed insertion of the CRISPR/cas-araC-Spectinomycin cassette into the tetracycline gene of RP4.

Recombineering was performed. Briefly, JKE201 cells with an lptA mutant allele, not targeted by the array, that contained apSIM5 recombineering plasmid were grown to an OD of 0.5 in LB supplemented with DAP and chloramphenicol. Then, the lambda RED system was induced at 42 C degrees for 15 min and the culture was placed on ice to prepare the cells for transformation by washing in cold MiliQ water. Cells were transformed with the PCR product at 18 kv and recovered for 12 h prior to plating on selective plates containing DAP and spectinomycin. The resulting recombinant clones were confirmed by PCR.

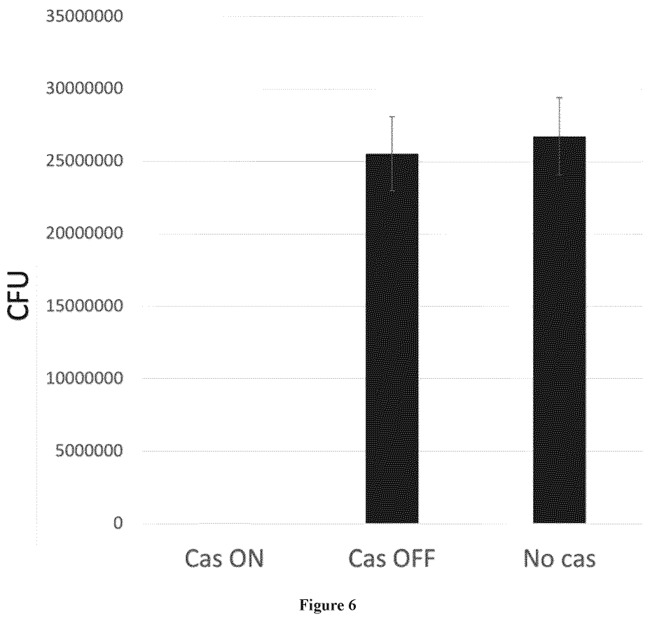

A positive clone as well as JKE201 with the wildtype RP4 plasmid (negative control without CRISPR/Cas) was grown in liquid LB with spectinomycin for 4 hrs. Wildtype MG1655 cells were grown to the same OD and mixed with the JKE201/RP4::Cas and JKE201/RP4 to allow plasmid transfer and amplification in this strain. The mixes were resuspended in LB without DAP to remove the initial JKE201 donor strain. The resulting population of MG1655 (newly formed plasmid donor cells) containing the plasmid were now plated on LB plates with and without arabinose (arabinose for CRISPR/cas induction) to active self-targeting and killing of donor cells by cutting the lptA target comprised by the donor chromosome. The population containing the wildtype RP4 plasmid was plated on the same media as a control. After overnight incubation, the surviving cells were enumerated and presented in FIG. 6.

The results depicted in FIG. 6 demonstrate that robust counterselection of a strain with one or more targets of interest can be done upon induction of CRISPR/cas. The engineered conjugative plasmid presented here, was able to discriminate between two closely related strains in a mixed culture, suggesting that it will be useful for precise donor strain removal in a microbiome context, such as in vivo in a human or animal subject. With additional targets added to the array, multiple targets can be hit simultaneously. A multi-target approach is the ideal implementation of a tight biocontainment strategy, essentially combining the two approaches of donor cell genome and plasmid targeting shown here, that will allow complete removal of the product (pathway) of interest from the microbiome in a controllable manner.

### Example 6: Illustrative Pathways Employing the Vectors, Cells and Methods of the Invention

Various metabolic pathway schemes can be envisaged where the invention functions. For example, the pathway may be in any environment comprising a microbiota, such as in a human or animal subject. Examples of schemes are provided in FIG. 7. The pathway scheme may function in the subject, such as confined to the microbiota (or even confined within the target cell that has received the vector nucleic acid encoding P1 and P2). Alternatively, part of the scheme (at least a part involving the target cell) takes place in the microbiota and another part takes place outside the microbiota in the environment or subject, such as where a metabolite is secreted from the target cell and is metabolized in the downstream part of the pathway scheme. In an embodiment. R may be produced as a metabolite (in the target cell or in a different cell, such as a different cell of the microbiota), thereby providing R to regulate P2 (and thus P1) expression in a feedback loop.

R may upregulate P2 expression, P2 downregulates P1 expression and optionally P1 is a component in a metabolic pathway (eg, in the microbiota or target cell or in a subject or environment comprising the microbiota or target cell) wherein a production product (X) of the pathway downstream from P1 causes regulation of P1 or P2 expression. See, for example, Scenarios 1, 3 and 4 (FIG. 7). In an embodiment, X is R or a precursor of R. In an embodiment, X regulates the first and/or second promoter. In an embodiment, X upregulates P1 or P2 expression. In an embodiment, X upregulates P1 or P2 expression.

R may upregulate P2 expression, P2 upregulates Pt expression and optionally P1 is a component in a metabolic pathway (eg, in the microbiota or target cell or in a subject or environment comprising the microbiota or target cell) wherein a production product (X) of the pathway downstream from P1 causes regulation of P1 or P2 expression. See, for example, Scenario 2 (FIG. 7). In an embodiment, X is R or a precursor of R. In an embodiment, X regulates the first and/or second promoter. In an embodiment, X upregulates P1 or P2 expression. In an embodiment, X upregulates P1 or P2 expression.

### Example 7: Vector Removal by CRISPR Self-Targeting in Bacteroides

### 1. Executive Summary

In this study we developed and tested a plasmid comprising a CRISPR/Cas system (which we call a CRISPR-guided vector (CGV)) that could be removed from a population of Bacteroides thetaiotaomicron using vector-borne spacers targeting the CGV itself (self-targeting CGV). The CGV harbored an inducible type I-B CRISPR/Cas operon originating from Clostridioides difficile alongside five CGV-targeting spacers. We demonstrated that upon induction, the CRISPR/Cas system caused the CGV to be removed from more than 99.9% of the Bacteroides population without killing the host cells in the process.

### Objectives

### Objective 1

Deliver a self-targeting CGV to Bacteroides thetaiotaomicron VPI-5482 (bSNP2978) via conjugation from an Escherichia coli donor to show that the CGV can be efficiently removed upon CRISPR/Cas induction.

### Objective 2

Demonstrate that self-targeting CGVs are removed from a population of Bacteroides thetaiotaomicron VPI-5482 (bSNP2978) without off-target effects.

### 2 Materials and Methods

2.1 Bacterial strains and growth conditions

Escherichia coli strains were grown at 37° C. in lysogeny broth (LB) at 250 RPM, or on solid LB-agar plates (made with 1.5% (w/v) agar). For plasmid selection, the medium was supplemented with ampicillin (Amp, 100 μg ml⁻¹) or spectinomycin (Spc, 400 μg ml⁻¹). For growth of auxotrophic strains, the medium was further supplemented with diaminopimelic acid (DAP, 40 μg ml⁻¹). Bacteroides thetaiotaomicron VPI-5482 (bSNP2978) only grows anaerobically but does not die under aerobic conditions. For this reason, the “M45 variable atmosphere workstation” (Don Whitley, Yorkshire, UK) was used and set to contain a mixture of gasses N₂, H₂, and CO₂ in the ratio 92:6:2. All solutions and media intended for Bacteroides were allowed to reduce under these conditions for ˜24 hours before being used.

All Bacteroides strains were routinely grown at 37° C. in Brain Heart Infusion (BHI) broth supplemented with cysteine (1 g 1⁻¹), hemin (5 mg⁻¹), and NaHCO₃ (0.2% (w/v)). This medium, denoted as BHIpp, is based on a recent Bacteroides protocol (Bacic & Smith, 2008). When growth on solid media was required, the BHIpp broth was further supplemented with agar (1.5% (w/v)). For plasmid selection, the medium was supplemented with erythromycin (Erm, 10 μg 1⁻¹). For counterselection of the E. coli donor strain, the BHIpp medium was supplemented with gentamicin (Gm, 30 μg⁻¹).

2.2 CGV design and assembly

CGV pSNP1599 is based on a shuttle plasmid, which can be delivered into the Bacteroides thetaiotaomicron VPI-5482 recipient strain (bSNP2978) via conjugation from an E. coli (bSNP3235)-based donor strain.

To construct pSNP1599, five spacers complementary to protospacers found within the plasmid itself (details found in Appendix C) were first assembled into a CRISPR array and then cloned into the shuttle plasmid. In the same cloning reaction, the type 1-B CRISPR/Cas operon from the Clostridioides dificile strain 630Δerm (denoted as CdCas) was also cloned into the plasmid. Expression of the vector-borne CRISPR/Cas system was regulated by a rhamnose-inducible promoter (Pᵣₕₐ), which allowed the plasmid removal to be controlled. Detailed information on the construction of pSNP1599 is found in Appendix B.

2.3 Conjugative transfer of CGV from E. coli to B. thetaiotaomicron

First, the bSNP3235-based donor strain carrying the plasmid of interest was grown aerobically in 30 ml LB medium supplemented with DAP while the bSNP2978 recipient was grown anaerobically in 5 ml BHIpp broth. Once the donor and recipient cultures reached optical densities (OD₆₀₀) of 0.4±0.2 and 0.15±0.05, respectively, they were mixed, using 25 ml of the donor and 2.5 ml of the recipient. The mixture was spun down (9000×g for 10 minutes), supernatant removed, and the pellet resuspended in 100 μl PBS. The resuspended pellet was spotted onto a BHIpp agar plate and allowed to incubate aerobically at 37° C. for 18 hours to facilitate conjugation.

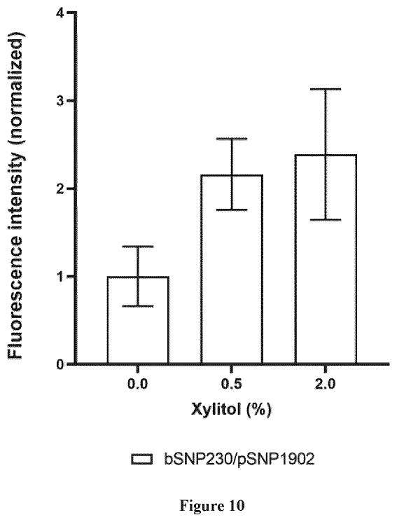

The mating spot was collected and resuspended in 500 μl pre-reduced BHIpp broth (from this point, the rest of the experiment was performed in the anaerobic work station). A 10-fold serial dilution was prepared in PBS (spanning dilutions 10¹ to 10¹) and then spotted onto selective media (BHIpp+Gm+Erm agar) with and without inducer. Plates were packed in plastics bags and allowed to incubate anaerobically at 37° C. for 2-3 days. Any transconjugant colony forming units (CFUs) emerging from the plates were enumerated. Two examples of plates after 3 days of incubation are given in FIG. 10.

2.4 Plasmid removal assay

A colony of bSNP2978 containing the intact self-targeting CGV pSNP1599 (verified by sequencing) was inoculated into 5 ml BHIpp broth supplemented with gentamicin and erythromycin and allowed to grow anaerobically overnight. The culture was then washed three times (each wash consisting of a spin-down at 4500×g for 2 min, removal of supernatant, and resuspension in 5-ml PBS) to remove all traces of erythromycin.

Two 5-μl aliquots of the washed culture were transferred to separate tubes containing 5 ml fresh pre-reduced BHIpp broth. One tube was supplemented with the inducer rhamnose (10 mM) while the other tube was given an equal volume of milliQ H₂O. The induced and non-induced cultures were allowed to grow anaerobically over the course of a day, with 200-p1 aliquots being taken from each culture at 0-, 1-, 3-, 6- and 24 hours after induction.

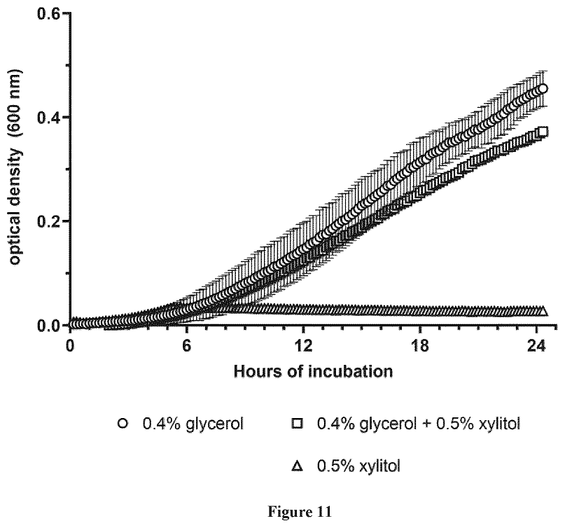

10-fold serial dilutions were prepared in PBS for each collected aliquot (spanning dilutions 10⁰ to 10⁻⁹) and spotted (2.5 μl per dilution) onto BHIpp agar plates either supplemented with gentamicin (non-selective plates) or gentamicin and erythromycin (selective plates). Plates were packed in plastic bags and allowed to incubate anaerobically for 2-3 days. CFUs were counted. Experiment was performed with biological triplicates. Two examples of plates after 3 days of incubation are given in FIG. 11.

### 3 Results

3.1 Estimating the efficacy of CRISPR-mediated CGV removal

A self-targeting CGV, denoted as pSNP1599, was generated, comprising a type I CRISPR/Cas system from C. difficile (CdCas) and five spacers which target the CGV itself. Upon induction with 30 rhamnose, the CdCas system is activated which lead to CRISPR targeting and, in turn, removal of the CGV.

The removal efficiency was tested by conjugating the CGV into the model strain Bacteroides thetaiotaomicron VPI-5482 (bSNP2978) via a mating with an E. coli (bSNP3235)-based donor strain and plating the mating mixture onto solid medium that selecting for the CGV with and without rhamnose. As a control, the empty plasmid pSNP1380 was conjugated via a separate mating.

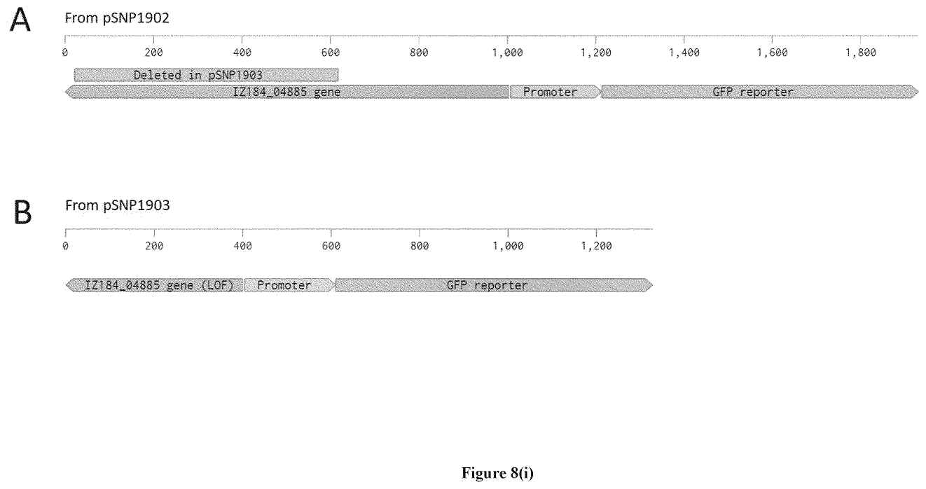

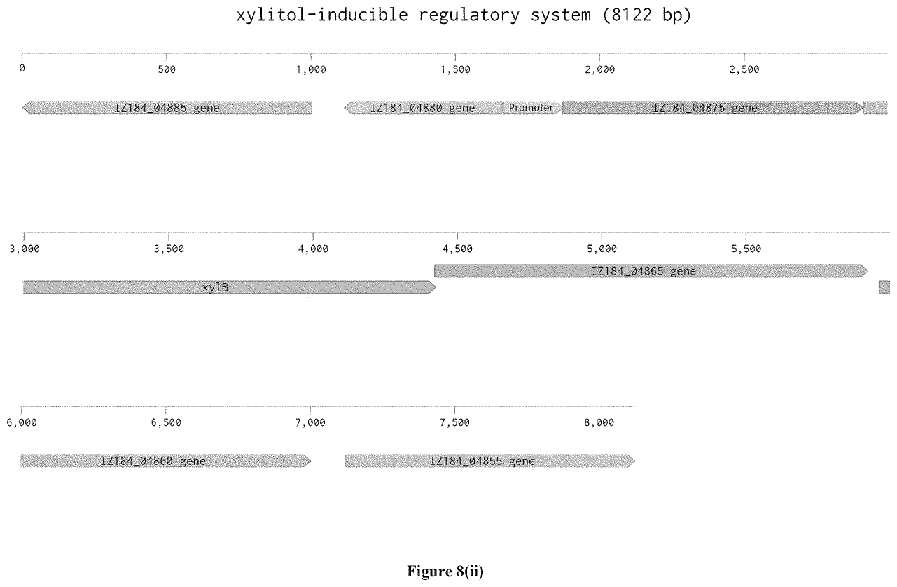

Conjugation experiments showed that the CGV was efficiently removed, as 1000-fold less transconjugants emerged on the selective media with induction compared to the plates without induction (˜10⁴ CFU/ml and ˜10⁷ CFU/ml, respectively) (FIG. 8). Conjugation of the empty control plasmid shows no difference in transconjugants regardless of induction.

3.2 Testing for off-target effects of self-targeting CGVs

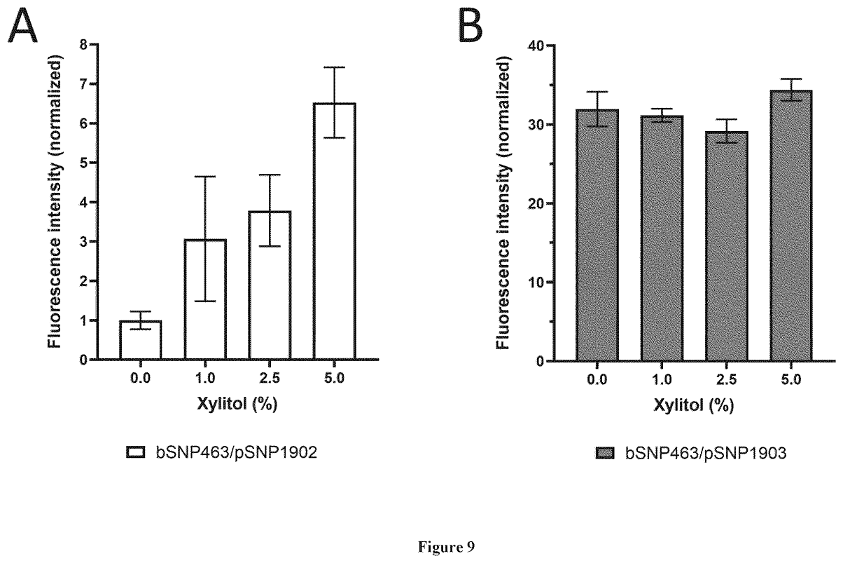

To test if the self-targeting CGV is removed without killing the Bacteroides host, we grew bSNP2978 (pSNP1599) with and without rhamnose (FIG. 9A, top). At various time points we took out aliquots, made serial dilutions, and spread them onto solid media plates with and without erythromycin (GCV antibiotic marker) (FIG. 9A, bottom), allowing us to estimate the sizes of the plasmid-harboring population and the total population, respectively.

One hour after induction, around 99% of the cells in the population lost their CGVs (FIG. 9B). This difference is gradually increased throughout the experiment. As expected, for the non-induced culture the number of cells was similar regardless of the selection, suggesting that our CGV was stable in Bacteroides unless we actively remove it by adding the inducer.

### 4 Conclusions

A new CGV carrying a self-targeting CRISPR/Cas system (CdCas) can efficiently was removed from more than 99.9% of cells in a population of B. thetaiotaomicron upon induction with rhamnose.

It was also demonstrated that survival and growth of the B. thetaiotaomicron cells was unaffected by the induced self-targeting CGV, suggesting that the CRISPR/Cas-mediated CGV removal had no off-target effects.

### Appendix A: Bacterial Strains Used in this Study

| Bacterial strain ID | Description | Reference or source |
|---|---|---|
| bSNP2522 | E. coli NEB ® 10-beta. | New England Biolabs |
| | Used as cloning strain. | (Catalog # C3020K) |
| bSNP2978 | Bacteroides thetaiotaomicron VPI-5482. | Deutsche Sammlung von |
| | Anaerobic bacterium known for being | Mikroorganismen und |
| | abundant in the human gut. | Zellkulturen GmbH |
| | | (DSM No. 2079) |

### Appendix B: Construction and Cloning of Self-Targeting CGV pSNP1599

Design and assembly of CRISPR array

A CRISPR array was designed by identifying five 37-bp protospacers in the backbone of the shuttle plasmid (details in Appendix C), each positioned adjacent to the consensus type I-B PAM sequence (5′-CCW-3′). A preliminary sequence similarity search (BLASTn, not shown) did not predict any off-targets for any of the five chosen spacers to the chromosome of bSNP2978. The repeat sequence (5′-GTTTTATATTAACTAAGTGGTATGTAAAT-3′ (SEQ ID NO: 5)) compatible with type I-B CRISPR/Cas system was chosen.

### Appendix C

| | |
|---|---|
| pSNP1599 | Based on shuttle plasmid, containing a |
| | 6.6-kb type I-B Cas operon from the C. |
| | difficile strain 630Δerm (CdCas) and a |
| | 359-bp CRISPR-array containing five |
| | 37-bp spacers which are designed to |
| | target the CGV itself. The entire locus |
| | was regulated by a rhamnose-inducible |
| | promoter and the consensus RBS sequence |
| | used by Bacteroides spp .. |

The five spacers are listed below:

| Target | |
|---|---|
| gene: | Spacer sequence (5′→3′): |
| mobB: | 5′-TTCCGGGGAACTGTACCGGGACAACTGGATAGGGAA |
| | A-3′(SEQ ID NO: 6) |
| ampR: | 5′-TGTTGTGCAAAAAAGCGGTTAGCTCCTTCGGTCCTC |
| | C-3′(SEQ ID NO: 7) |
| ermG: | 5′-AACTATCAAATAGTAAATGATGATATACTGAAATTT |
| | A-3′(SEQ ID NO: 8) |
| P_rha: | 5′-TCAAAACCAATGTAATATTCCGTCCATCCGGTCTGC |
| | C-3′(SEQ ID NO: 9) |
| pSC101: | 5′-GTTCTATTAGGTGTTACATGCTGTTCATCTGTTACA |
| | T-3′(SEQ ID NO: 10) |

### Example 8: Evaluation of the Xylitol-Inducible Promoter from Morganella morganii

### Executive summary

A predicted xylitol-inducible promoter originating from the bacterium Morganella morganii was cloned and investigated within various Escherichia coli strain backgrounds.

It was demonstrated that the promoter is repressed by a transcriptional regulator protein IZ184_04885 and that repression could be lifted either gradually by adding xylitol to the growth medium or completely by mutating the transcriptional regulator. Sensitivity of the promoter towards xylitol increased when the xylitol ABC transporter system was included as well.

### Objectives

### Objective 1

Demonstrate that the repression of the xylitol-inducible promoter can be lifted by adding increasing amounts of xylitol to the growth medium.

### Objective 2

Demonstrate that the repression of the xylitol-inducible promoter is avoided by mutating the transcriptional regulator protein IZ184_04885.

### Objective 3

Investigate whether expression of the xylitol ABC transporter increases the sensitivity of the xylitol-inducible promoter towards xylitol in the medium.

### Materials and Methods

### Bacterial Strains and Growth Conditions for Plasmid Cloning

All strains of Escherichia coli used in this study are listed in Table 5, Appendix A. For cloning, E. coli strains were grown at 37° C. in lysogeny broth (LB) at 250 RPM or on solid LB-agar plates (made with 1.5% (w/v) agar). For plasmid selection, the medium was supplemented with tetracycline (10 μg ml⁻¹) and/or spectinomycin (Spc, 100 μg ml⁻¹).

### Plasmid Construction

GFP reporter plasmids

Based on an annotated genome sequence of Morganella morganii strain ZJG812 (Genome ID: CP064831.1), a 1213-bp DNA sequence encoding the xylitol-inducible regulatory system (excluding the sugar isomerase gene IZ184_04880) was defined and ordered as a gBlock fragment from Integrated DNA Technologies™ (Coralville, IA, USA). The gBlock DNA fragment was further amplified by PCR using the CloneAmp HiFi PCR Premix from Takara Bio (Mountain View, CA, USA) with primers SEM1770 and SEM1771. In parallel, the backbone of the plasmid pSNP1248 was PCR amplified using the CloneAMP HiFi PCR premix with primers SEM1282 and SEM1769, yielding a 32.50-bp DNA fragment that contain the fluorescent reporter gene (gfp), a low copy-number origin of replication (cloDF13) and a spectinomycin resistance marker (Spc®). The full list of primers used in this study is found in Table 6, Appendix B.

The amplified DNA fragments were routinely treated with DpnI (1 hour at 37° C. followed by 20 minutes at 80° C.) and purified by gel extraction using the MinElute kit from Qiagen (Hilden, Germany) according to the manufacturer's instructions. The purified regulatory system DNA was inserted into the linearized pSNP1248 backbone with the In-Fusion® HD Cloning kit from Takara Bio according to the manufacturer's instruction. Chemically competent bSNP2480 cells were transformed with the DNA from the In-Fusion® reaction mixture. Transformed cells were spread onto LB+Spc¹⁰⁰ agar plates and allowed to incubate overnight. Plasmids were purified from individual colonies using the QIAprep Spin Miniprep Kit from Qiagen according to the manufacturer's instructions and followingly verified by Sanger sequencing. The positive clone was registered as plasmid pSNP1902. In addition, a serendipitous clone containing a 595-bp deletion located within the transcriptional regulator open reading frame (ORF) (IZ184_04885) was identified and registered as plasmid pSNP1903.

xylitol ABC transporter plasmid

The 3728-bp.xylABC operon from Morganella morganii strain ZJG812 (Genome ID: CP064831.1) was ordered as two gBlock DNA fragments from Integrated DNA Technologies™. The plasmid backbone was prepared by PCR amplification of plasmid pSNP639 using the CloneAmp HiFi PCR Premix with primers SEM1814 and SEM1815. The amplified fragment was DpnI treated and purified by gel extraction, following the same method as described in section 3.2.1.

The two gBlock DNA fragments and the linearized backbone DNA were fused together using the In-Fusion® HD Cloning kit from Takara Bio according to the manufacturer's instruction. Chemically competent bSNP2522 cells were transformed with the DNA from the In-Fusion@ reaction mixture. Transformed cells were spread onto LB+Tet¹⁰ agar plates and allowed to incubate overnight. Plasmids were purified from individual colonies using the QIAprep Spin Miniprep Kit from Qiagen according to the manufacturer's instructions and followingly verified by Sanger sequencing. The positive clone was registered as plasmid pSNP1939. The full list of plasmids used in this study is found in Table 7, Appendix C.

### GFP Expression Assay for Characterization of Xylitol-Inducible Promoter

### Preparation of Model Strains

The constructed GFP reporter plasmids (pSNP1902 and pSNP1903), the xylitol ABC transporter-containing plasmid (pSNP1939) as well as the control plasmids (see Table 7, Appendix C) were transformed individually or in combination into the model strains E. coli MG1655 (bSNP230) and a probiotic E. coli isolate (bSNP463) via electroporation. Transformed cells were spread onto LB+Spc¹⁰⁰-, LB+Tet¹⁰- or LB+Spc¹⁰⁰+Tet¹⁰⁰ agar plates depending on whether the cells were transformed with the GFP reporter plasmid, xylitol ABC transporter plasmid, or both, respectively. The plates were allowed to incubate overnight. Three colonies of each strain were picked and used as biological triplicates in the ensuing 24-hour gfp expression assay.

24-hour gfp expression assay Triplicate colonies were inoculated into 5-ml LB medium or 5-ml M9 minimal medium broth (recipe found in Appendix D) supplemented with Tet (10 μg ml⁻¹) and/or Spc (100 μg ml⁻¹) and allowed to incubate overnight (˜16 hours) at 37° C. and 250 RPM. Cultures would have reached optical densities (OD₆₀₀) of 0.8±0.3 in M9 minimal medium and 1.4±0.2 in LB medium.

Each overnight culture was then diluted 100× in fresh medium supplemented with appropriate antibiotics (as above) and distributed into 3-6 new cultures which were given xylitol at different concentrations within the range of 0% to 5% (the exact concentrations of xylitol are listed in the related figures in section 4). The cultures were then transferred as 200-μl aliquots into a black 96-well microtiter plate with transparent bottom. To normalize the fluorescence emissions, a row of wells in the plate were dedicated to cells that harbor the control plasmids pSNP958 and pSNP1617 which do not encode gfp (to estimate autofluorescence) and another row dedicated to media without any cells (to estimate background fluorescence). The microtiter plate was sealed with a Breath-Easy® sealing membrane from Merck (Whitehouse station, NJ, USA) and placed in the Synergy H1 microplate reader from Agilent Biotek (Winooski, VT, USA) programmed to incubate for 24 hours at 37° C. with a constant agitation. Both OD₆₀₀ and GFP fluorescence (excitation at 485 nm, emission at 516 nm, gain of 90) were measured every 10 minutes throughout the incubation. The recorded fluorescence emissions were normalized according to the corresponding cell densities and corrected for the background and autofluorescence levels.

### Results

Criteria and choice of the inducible promoter We have developed a biocontainment strategy using CRISPR/Cas systems as off-switches of our production circuits or strains. This requires that the CRISPR/Cas system is under tight transcriptional control from a promoter which is activated upon addition of an external inducer molecule.

Within the field of molecular microbiology, operons of genes related to the conversion/utilization of sugars may be induced by the sugars themselves as they bind and influence the transcriptional repressor proteins. For the present study, it was decided to investigate a negatively inducible promoter predicted to be induced by xylitol. This 5-carbon sugar alcohol has several advantages. First of all, xylitol is generally regarded as safe for human consumption by the FDA (Xiang et al., 2021). Furthermore, over half of ingested xylitol is not adsorbed by human cells instead reaching the gastrointestinal tract where it is taken up by the microbiome (Livesey, 2003), making this useful to control our switches in the GI tract. Finally, xylitol is a known metabolite for some bacteria: It is either taken up directly through an ABC-type transporter complex (Madigan et al., 2015) and/or generated through reduction of the corresponding sugar D-xylose. Subsequently, xylitol can be dehydrogenated and phosphorylated to xylulose-5-phosphate which is further catabolized in the pentose phosphate pathway.

From a dated PhD thesis (Gallo, 1991), we found an operon in the genome of the bacterium Morganella morganii strain ZJG812 which contain genes related to xylitol uptake and metabolism (details summarized in FIG. 8(ii)). This operon is located downstream from a ˜1800-bp regulatory region which, among other elements, consist of a negatively inducible promoter and a cognate transcriptional regulator protein IZ184_04885 (details in Appendix E) which xylitol is suggested to bind.

Estimating promoter activity in response to xylitol in growth medium

For this study. two reporter plasmids were constructed consisting of a green fluorescent protein (GFP) reporter gene located downstream from the xylitol-inducible regulatory system either without (pSNP1902, FIG. 8(i)A) or with (pSNP1903. FIG. 8(ii)B) a loss-of-function deletion located in the ORF of the transcriptional regulator.

The reporter plasmids consist of a green fluorescent protein (GFP) reporter gene located downstream from the predicted xylitol-inducible promoter which in turn is controlled by the LacI family DNA-binding transcriptional regulator (IZ184_04885 gene). Two reporter plasmids are made, either (A) with a full-length transcriptional regulator (plasmid pSNP1902) or (B) with a 595-bp loss-of-function (LOF) deletion within the ORF of the transcriptional regulator (plasmid pSNP1903).

The GFP reporter plasmids pSNP1902 and pSNP1903 were transformed into the probiotic isolate strain bSNP463 and tested by measuring the optical density (OD₆₀₀) and green fluorescence emitted from cultures after a 24-hour incubation in the presence of increasing amounts of xylitol. The results from these tests are summarized in FIG. 9. It is observed how the fluorescence intensity per cell increases up to 6-fold as xylitol is added to the strains which harbor the GFP reporter plasmid with the full-length transcriptional regulator (FIG. 9A). When the transcriptional regulator is mutated by a 595-bp deletion (FIG. 9B), the basal fluorescence intensity is approximate 30-fold higher than that from the strains with the full-length transcriptional regulator and the fluorescence intensity does not change when xylitol is added. This suggest that xylitol induces expression by lifting the repression that the transcriptional regulator exerts on the promoter.

The test of the GFP reporter plasmid with the full-length transcriptional regulator (pSNP1902) was repeated in a minimal medium broth using the E. coli strain MG1655 (bSNP230) which gave similar results (as shown in FIG. 10). Note that the applied concentrations of xylitol were lower for these tests as it was shown that increasing concentrations of xylitol slowed down the growth rate of bacteria when grown on minimal medium supplemented with glycerol. Furthermore, when all other carbon sources than xylitol were omitted from the minimal medium, the E. coli strains did not grow at all (as illustrated from the growth profiles shown in FIG. 11). This is likely because E. coli strains generally lack the xylose reductase and xylitol dehydrogenase typically necessary for xylitol metabolism (Ge et al., 2018). This may be useful for more precise or lasting control by xylitol when the strain cannot metabolise xylitol. Use of lower doses of xylitol may be possible when the strain cannot metabolise xylitol.

Testing whether the presence of the xylitol ABC transporter increases the sensitivity of the xylitol-inducible promoter

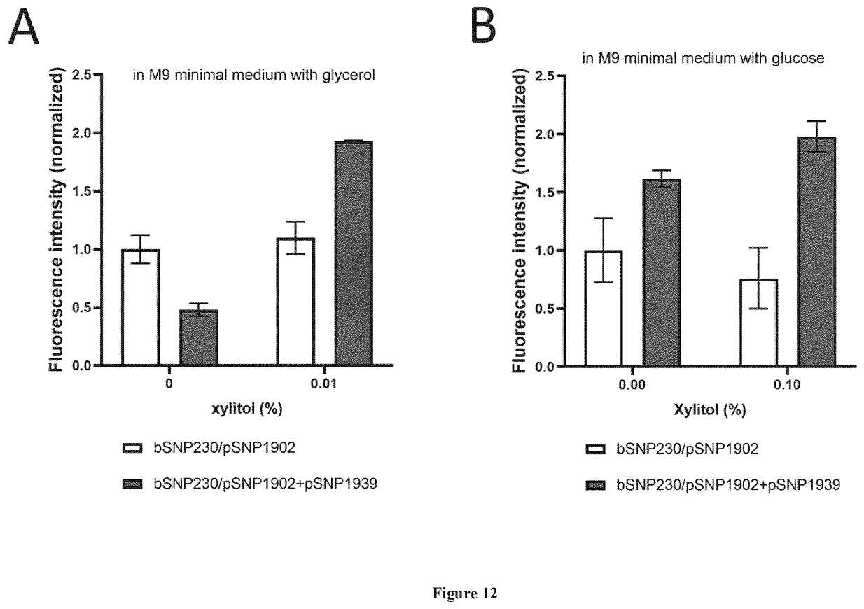

The activity of a negatively inducible promoter will depend on the import of the inducer molecule into the cell. In this context, it was hypothesized that expression of the predicted xylitol ABC transporter operon (mentioned in section 4.1) would allow more xylitol to enter the cell and hence increase the sensitivity of the promoter. To test this, plasmid pSNP1939 was transformed into the bSNP230-construct already containing the GFP reporter plasmid pSNP1902 and tested by the same 24-hour assay as used previously. For these experiments, the well-defined M9 minimal media were used either with glycerol or glucose as the carbon source for growth. Glucose was chosen as it is the preferred sugar for bacterial growth, but it might also have an inhibitory effect on promoters which regulate alternative metabolic pathways such as those related to xylitol. Glycerol was chosen as it had previously been shown by us (data not shown) not to influence the expression from the xylitol-inducible promoter. The results are summarized in FIG. 12.

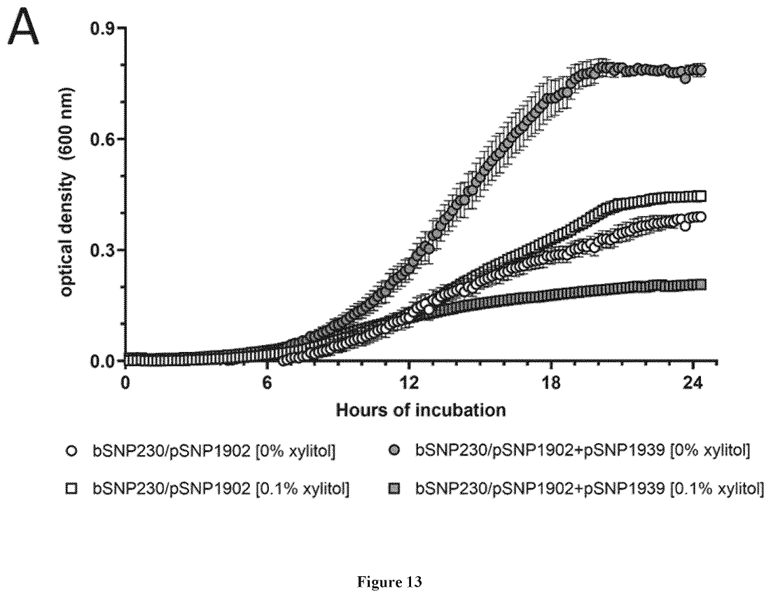

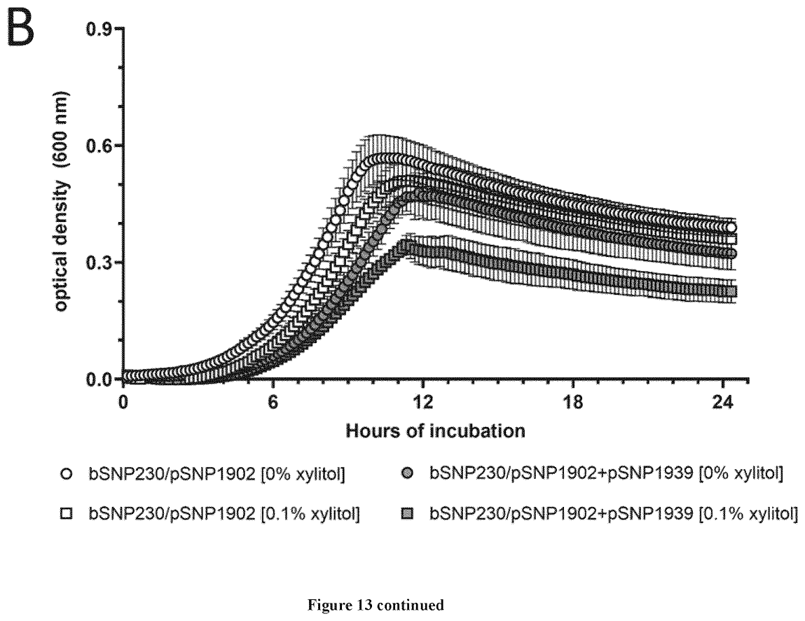

We used bSNP230 strains containing the GFP reporter plasmid (pSNP1902) either with or without a co-resident plasmid containing the xylitol ABC transporter (pSNP1939) were grown for 24-hours in minimal medium supplemented with 0.4% (v/v) glycerol (A) or 0.4% (w/v) glucose (B) in the presence or absence of xylitol. The cell-density adjusted fluorescence emission after 24 hours of incubation are shown above. Error bars indicate standard deviations based on two (A) and three (B) biological replicates, respectively. The corresponding growth curves are shown in FIG. 13.

At these low concentrations of xylitol, the strain bSNP230/pSNP1902 did not exhibit any change in fluorescence intensity (but higher concentrations of xylitol in a different experiment may lead to increased fluorescence). For the strain bSNP230/pSNP1902p+pSNP1939, however, it was observed that the fluorescence intensity increased as xylitol was added—an effect which was more pronounced when the minimal medium was supplemented with glycerol (FIG. 13A) rather than glucose (FIG. 13B). These data suggest that the presence of the xylitol ABC transporter increases the sensitivity of the reporter plasmid-carrying strains towards xylitol in the growth medium.

### CONCLUSIONS

The xylitol-inducible promoter originating from Morganella morganii is repressed by the transcriptional regulator protein IZ184_04885 when tested in various E. coli strain backgrounds. This repression was lifted gradually by adding xylitol to the growth medium or fully by mutating IZ184_04885. The sensitivity of the promoter towards xylitol in the growth medium was further enhanced by including the xylitol ABC transporter system.

### REFERENCES

Gallo, M. A. (1991). Molecular characterization of xylitol catabolic pathways in the Enterobacteriaceae. Conrell University.
Ge, X., Chang, C., Zhang, L., Cui, S., Luo, X., Hu, S., Qin, Y., & Li, Y. (2018). Conversion of Lignocellulosic Biomass Into Platform Chemicals for Biobased Polyurethane Application. In Advances in Bioenergy (Vol. 3, pp. 161-213). Elsevier. https://doi.org/i0.1016/BS.AIBE.2018.03.002
Livesey, G. (2003). Health potential of polyols as sugar replacers, with emphasis on low glycaemic properties. Nutrition Research Reviews, 16(2), 163-191. https://doi.org/10.1079/nrr200371
Madigan, M. T., Martinko, J. M., Bender, K. S., Buckley, D. H., Stahl, D. A., & Brock, T. (2015). Microbial Metabolism. In Brock Biology of Microorganisms (14th ed.). Pearson.
Xiang, S., Ye, K., Li, M., Ying, J., Wang, H., Han, J., Shi, L., Xiao, J., Shen, Y., Feng, X., Bao, X., Zheng, Y., Ge, Y., Zhang, Y., Liu. C., Chen, J., Chen, Y., Tian, S., & Zhu, X. (2021). Xylitol enhances synthesis of propionate in the colon via cross-feeding of gut microbiota. Microbiome, 9(1), 1-21. https://doi.org/10.1186/s40168-021-01029-6

### APPENDICES

### Appendix A: Bacterial Strain Backgrounds

TABLE 5
Bacterial strain backgrounds used in this study

| Strain ID | Description | Reference or source |
|---|---|---|
| bSNP230 | E. coli MG1655 | Our collection |
| bSNP463 | E. coli probiotic isolate | Our collection |
| bSNP2480 | E. coli Takara Stellar | Takara Bio |
| | | (Mountain View, CA, USA), |
| | | Cat#636763 |
| bSNP2522 | E. coli NEB 10-beta | New England Biolabs |
| | | (Ipswich, MA, USA), |
| | | Cat#C3019H |

### Appendix B: Primers

TABLE 6
Relevant primers and their sequences

| Primer | |
|---|---|
| ID | Sequence (5′→3′) |
| SEM1282 | GCTAGCAGAGTTTGTAGAAACGC (SEQ ID NO: 11) |
| SEM1769 | ATGCGTAAAGGCGAAGAACTGT (SEQ ID NO: 12) |
| SEM1770 | TTTCTACAAACTCTGCTAGCTTAAGATTTGAGGAGACCG |
| | CGCAC (SEQ ID NO: 13) |
| SEM1771 | AGTTCTTCGCCTTTACGCATGATCATTTCCTTTCATTAA |
| | AATGATATCGATATC (SEQ ID NO: 14) |
| SEM1814 | GACCTCCGGGTATTCTGATGCATTGAATTCCTCCTGCTA |
| | GCCT (SEQ ID NO: 15) |
| SEM1815 | ACTGGATGCCCGTAAATAAATGTTGTTTGTCGGTGAACT |
| | CTGC (SEQ ID NO: 16) |

### Appendix C: Plasmids

TABLE 7
Plasmids used in this study

| Vector ID | Description | Reference or source |
|---|---|---|
| pSNP639 | Plasmid backbone used for construction of | Our collection |
| | pSNP1939. Encodes a tetracycline resistance marker | |
| | (Tet^{R}). | |
| pSNP958 | Plasmid used as negative control in fluorescence | Our collection |
| | analyses because it has the same resistance marker | |
| | (Spc^{R}) and origin of replication as pSNP1248- | |
| | derived plasmids but lacks the gfp reporter gene. | |
| pSNP1248 | Plasmid encoding the gfp reporter gene. Plasmids | Our collection |
| | pSNP1902 and pSNP1903 are based on this | |
| | backbone. Encodes a spectinomycin resistance | |
| | marker (Spc^{R}). | |
| pSNP1617 | Plasmid used as a negative control because it has the | Our collection |
| | same resistance marker (Tet^{R}) and origin of | |
| | replication as pSNP1939 but lacks the xylitol ABC | |
| | transporter. | |
| pSNP1902 | GFP reporter plasmid containing the xylitol- | This study |
| | inducible regulatory system originating from | |
| | Morganella morganii but without the sugar | |
| | isomerase gene (IZ184_04880). Encodes a | |
| | spectinomycin resistance marker (Spc^{R}). | |
| pSNP1903 | Same as p1902 except that the repressor protein | This study |
| | ORF contains a 595-bp deletion. | |
| pSNP1939 | Plasmid encoding the xylitol ABC transporter | This study |
| | system (xylABC) originating from Morganella | |
| | morganii. Encodes a tetracycline resistance marker | |
| | (Tet^{R}). | |

### Appendix D: M9 Minimal Medium Recipe

The M9 minimal medium used in this study report consists of a medium salts solution supplemented with a carbon source (glucose or glycerol). B1 vitamin, magnesium, calcium and a source of iron.

The medium salts solution is a composition of four crucial salts. We recommend preparing it as a 5× stock solution according to Table 8 below:

TABLE 8
5x M9 minimal salts solution

| | | |
|---|---|---|
| Component | Amount added per liter | Final concentration |

| | | | | |
|---|---|---|---|---|
| Na₂HPO₄ | 30 | g | 210 | mM |
| KH₂PO₄ | 15 | g | 110 | mM |
| NaCl | 5 | g | 428 | mM |
| NH₄Cl | 2.5 | g | 93 | mM |

Fill a beaker with ~800 ml distilled H₂O and add the specified amounts of the four salts while the solution is thoroughly mixed by a magnetic stir bar. Once the components have dissolved fill up with distilled H₂O to 1000 ml. Sterilize by autoclaving the solution at 121° C. for 15 minutes. The 5x M9 salts solution can be stored at room temperature for up to a year.

All remaining stock solutions required to make the M9 medium salts solution are prepared routinely by weighing the appropriate amount of the component, dissolving it in distilled H₂O under constant agitation and filter-sterilizing it (pore size=0.2 μm). Once prepared, mix the stock solutions together according to the instructions from Table 9 below:

TABLE 9
Recipe for M9 minimal medium for E. coli growth

| | Amount added per | | | |
|---|---|---|---|---|
| Stock solution | liter of M9 medium | | Final concentration | |
| 5x M9 salts solution | 200 | ml | 42 mM | (Na₂HPO₄) |
| | | | 27 mM | (KH₂PO₄) |
| | | | 85.6 mM | (NaCl) |
| | | | 18.6 mM | (NH₄Cl) |
| 20% (v/v) glycerol | 20 | ml | 0.4% | |
| or | | | | |
| 20% (w/v) glucose | | | | |
| 2M MgSO₄•7H₂O | 2 | ml | 4 | mM |
| 0.1M CaCl₂•2H₂O | 2 | ml | 0.2 | mM |
| 0.05M FeCl₂•6H₂O | 1 | ml | 0.05 | mM |
| 1% (w/v) Thiamin-HCl | 1 | ml | 0.001% | (w/v) |

| | | |
|---|---|---|
| Deionized water | To 1000 ml | — |

Fill a beaker with ~600 ml distilled H₂O and add the specified volumes of stock solutions. The 1x M9 minimal medium solution can be stored at room temperature for up to a month.

### Appendix E: DNA- and Amino Acid Sequence of the Lacd Family DNA-Binding Transcriptional Regulator IZ184_04885

IZ184 04885 DNA sequence (5′-→3′)
(SEQ ID NO: 1)
ttggcggaaccccggacacagaaagtcacactggaaaatgtggccgctat
tgcgaatgtcagtaagatcaccgcatcccgcgcattttcgcagccggata
aggttcacccggaaactctcaggcgtattctcgatgccgcagataaaatc
ggttatgtggtgaatgcggcggcacgcagcctgcgcgcgaaatcctcccg
taccatcggtattgtcagcccggacatgagcaacccgtttttcgggggct
ggccaagcgcatcaccctggaagcctataacgccgggtatgacacgctga
tgtttgactcctatgaatcacgtgaaaatgaagcgcgtatcattgataag
ctgattggttacaacgtggatgccatcattctgtccgttgtctccgccga
aagggtgtatcgcccggcgtatatgaaacagctggaactgctcaatattc
cggtgatcctggttgaccgtgagctggatgccaaggcatgcagcggcgtt
tacatcgataatctcgactgcggattgcaggcagggcgctatctgctgtc
acaaaaagcggataacgtggtgattgtctccggcccggaagattccaatg
tggcccaggatcgcgtaaccggcatggtggccggtctgcacggccaggtc
agcagcgtcaatgtgctgcatgcggacttcctgatggatgaagcctttaa
agtcaccgaccattacctgaaataccatcccgcgccggattattttgtcg
gctgcaataaccagatctcactcgggattatcaaagcctgtatccgccac
aatctgatcccgcaaaaagatgtctcgctgttcagcatcgatgaagtctc
tcatgccgatatttacggattcaatttcccgtgcatttctcatgatttac
aggaaattgcctggcaggccattaacatggcagtacggcgggcgacggat
cgcagcgccccggcgagcaaggtggttgtgcgcggtctcctcaaatctta
a
IZ184 04885 amino acid sequence
(SEQ ID NO: 2)
MAEPRTQKVTLENVAAIANVSKITASRAFSQPDKVHPETLRRILDAADKI
GYVVNAAARSLRAKSSRTIGIVSPDMSNPFFGGLAKRITLEAYNAGYDTL
MFDSYESRENEARIIDKLIGYNVDAIILSVVSAERVYRPAYMKQLELLNI
PVILVDRELDAKACSGVYIDNLDCGLQAGRYLLSQKADNVVIVSGPEDSN
VAQDRVTGMVAGLHGQVSSVNVLHADFLMDEAFKVTDHYLKYHPAPDYFV
GCNNQISLGIIKACIRHNLIPQKDVSLFSIDEVSHADIYGFNFPCISHDL
QEIAWQAINMAVRRATDRSAPASKVVVRGLLKS

### Appendix F: Further Sequences

Promoter sequence (5′-→3′)
(SEQ ID NO: 3)
cctgaatgatatcgttatcattatagccttgtcacagatagcaaagatgt
gacccggaagaccctttgccgaatactacatgagtagatcagactttatt
taataaaagtttaaccatcatcacataattataataaatattgctctatt
atccgcatcatagtaatgatatcgatatcattttaatgaaaggaaatgat
c
Sequence comprising (in 5′ to 3′ direction) the
sequence of the repressor, isomerase and promoter
of Morganella morganii strain ZIG812 (SEQ ID NO:
4)

This sequence can be used in the invention and provides the repressor and promoter.

ttaagatttgaggagaccgcgcacaaccaccttgctcgccggggcgctgc
gatccgtcgcccgccgtactgccatgttaatggcctgccaggcaatttcc
tgtaaatcatgagaaatgcacgggaaattgaatccgtaaatatcggcatg
agagacttcatcgatgctgaacagcgagacatctttttgcgggatcagat
tgtggcggatacaggctttgataatcccgagtgagatctggttattgcag
ccgacaaaataatccggcgcgggatggtatttcaggtaatggtcggtgac
tttaaaggcttcatccatcaggaagtccgcatgcagcacattgacgctgc
tgacctggccgtgcagaccggccaccatgccggttacgcgatcctgggcc
acattggaatcttccgggccggagacaatcaccacgttatccgctttttg
tgacagcagatagcgccctgcctgcaatccgcagtcgagattatcgatgt
aaacgccgctgcatgccttggcatccagctcacggtcaaccaggatcacc
ggaatattgagcagttccagctgtttcatatacgccggggatacaccctt
tcggcggagacaacggacagaatgatggcatccacgttgtaaccaatcag
cttatcaatgatacgcgcttcattttcacgtgattcataggagtcaaaca
tcagcgtgtcatacccggcgttataggcttccagggtgatgcgcttggcc
agcccgccgaaaaacgggttgctcatgtccgggctgacaataccgatggt
acgggaggatttcgcgcgcaggctggtgccgccgcattcaccacataacc
gattttatctgcggcatcgagaatacgcctgagagtttccgggtgaacct
tatccggctgcgaaaatgcgcgggatgcggtgatcttactgacattcgca
atagggccacattttccagtgtgactttctgtgtccggggttccgccaat
tttctctccttagcagcaacgtatcattttataccattataatatcatct
aaatcgattcagtcacgtgaaaaacacgctttttattacttaaaccgaca
ctaacaactttttaacactcgagtctttttatccgcggatcggtaaaaat
gtcgtattcatcggtgttttctgtggagaactccgacaccaccgcgccct
gcggtccggctttaaaccagtgggggtatccggcatgatggtgtactgct
ctcccgggcgcagcagaataaagcggtcacaggtgtaccattcctcatcc
ccctgcggcggacggcagaccggattgccctgagcatcctgattcagtgt
cagcgccggatcatccacaaacagatacacctcaccccagcggcagcgga
aagtttcctgcttacccggccggtctttgtacggcggatgacggtgctcc
ggacaggtctggtcagcaaagagaaccagctctttggcgcagtacagcgg
gctgttgcagtacgtgagcagttgcaggccagagcggggataatccggca
gattaaagtgagcaatttcaatatgctgctgttccgcttctgtcagaatg
atatgtgcctgttccagaaccgccagtgattccgcgatatacggctgttt
attcatctgcctctctcctgaatgatatcgttatcattatagccttgtca
cagatagcaaagatgtgacccggaagaccctttgccgaatactacatgag
tagatcagactttatttaataaaagtttaaccatcatcacataattataa
taaatattgctctattatccgcatcatagtaatgatatcgatatcatttt
aatgaaaggaaatgatc

TABLE 1
Example Cell Genera, Species & Strains
Each carrier cell may be a cell of a genus or species disclosed in
this table and/or target cell may be a cell of a genus or species
disclosed in this table; the genera of the carrier and target cells
may be the same of different; the species of the carrier and target
cells may be the same of different.

Acidovorax avenae subsp. cattleyae
Acidovorax avenae subsp.
Acidovorax konjaci
Acidovorax valerianellae
Agrobacterium
Agrobacterium larrymoorei
Agrobacterium radiobacter
Agrobacterium rhizogenes
Agrobacterium rubi
Agrobacterium tumefaciens
Agrobacterium vitis
Arthrobacter
Arthrobacter ilicis
Bacillus
Bacillus megaterium
Bacillus megaterium pv. cerealis
Bacillus pumilus
Brenneria
Brenneria alni
Brenneria nigrifluens
Brenneria quercina
Brenneria rubrifaciens
Brenneria salicis
Burkholderia
Burkholderia andropogonis
Burkholderia caryophylli
Burkholderia cepacia
Burkholderia gladioli
Burkholderia gladioli pv. agaricicola
Burkholderia gladioli pv. alliicola
Burkholderia gladioli pv. gladioli
Burkholderia glumae
Burkholderia
Clavibacter
Clavibacter michiganensis
Clavibacter michiganensis subsp.
Clavibacter michiganensis subsp. michiganensis
Clavibacter michiganensis subsp. nebraskensis
Clavibacter michiganensis subsp. sepedonicus
Clavibacter michiganensis subsp. tessellarius
Clavibacter rathayi
Clavibacter toxicus
Clavibacter tritici
Clavibacter xyli
Clavibacter xyli subsp. cynodontis
Clavibacter xyli subsp. xyli
Clostridium
Clostridium puniceum
Corynebacterium
Corynebacterium betae
Corynebacterium beticola
Corynebacterium fascians
Corynebacterium flaccumfaciens
Corynebacterium flaccumfaciens pv. betae
Corynebacterium flaccumfaciens pv. flaccumfaciens
Corynebacterium flaccumfaciens pv. oortii
Corynebacterium flaccumfaciens pv. poinsettiae
Corynebacterium flaccumfaciens subsp.
Corynebacterium flaccumfaciens subsp. flaccumfaciens
Corynebacterium flaccumfaciens subsp. oortii
Corynebacterium flaccumfaciens subsp. poinsettiae
Corynebacterium ilicis
Corynebacterium insidiosum
Corynebacterium iranicum
Corynebacterium michiganense
Corynebacterium michiganensis pv. insidiosus
Corynebacterium michiganensis pv. iranicum
Corynebacterium michiganense pv. nebraskense
Corynebacterium michiganense pv. rathayi
Corynebacterium michiganense pv. sepedonicum
Corynebacterium michiganense pv. tritici
Corynebacterium michiganense subsp. insidiosum
Corynebacterium michiganense subsp.
Corynebacterium michiganense subsp. nebraskense
Corynebacterium michiganense subsp. sepedonicum
Corynebacterium michiganense subsp. tessellarius
Corynebacterium oortii
Corynebacterium
Corynebacterium rathayi
Corynebacterium sepedonicum
Corynebacterium tritici
Curtobacterium
Curtobacterium flaccumfaciens
Curtobacterium flaccumfaciens pv.
Curtobacterium flaccumfaciens pv. flaccumfaciens
Curtobacterium flaccumfaciens pv. ilicis
Curtobacterium flaccumfaciens pv. oortii
Curtobacterium flaccumfaciens pv. poinsettiae
Dickeya
Dickeya chrysanthemi
Dickeya chrysanthemi pv. chrysanthemi
Dickeya chrysanthemi pv. parthenii
Dickeya dadantii
Dickeya dianthicola
Dickeya dieffenbachiae
Dickeya paradisiaca
Dickeya zeae
Enterobacter
Enterobacter agglomerans
Enterobacter cancerogenus
Enterobacter cloacae
Enterobacter cloacae subsp. dissolvens
Enterobacter nimipressuralis
Enterobacter pyrinus
Erwinia
Erwinia alni
Erwinia amylovora.
Erwinia amylovora pv. pyri
Erwinia ananatis corrig.
Erwinia ananatis pv. ananatis
Erwinia ananas pv. uredovora
Erwinia cacticida
Erwinia cancerogena
Erwinia carnegieana
Erwinia carotovora
Erwinia carotovora pv. atroseptica
Erwinia carotovora pv. carotovora
Erwinia carotovora subsp. atroseptica
Erwinia carotovora subsp. carotovora
Erwinia carotovora subsp. betavasculorum
Erwinia carotovora subsp. odorifera
Erwinia carotovora subsp. wasabiae
Erwinia chrysanthemi
Erwinia chrysanthemi pv. chrysanthemi
Erwinia chrysanthemi pv.
Erwinia chrysanthemi pv. dieffenbachiae
Erwinia chrysanthemi pv. paradisiaca
Erwinia chrysanthemi pv. parthenii
Erwinia chrysanthemi pv. zeae
Erwinia cypripedii
Erwinia dissolvens
Erwinia herbicola
Erwinia herbicola f. sp.
Erwinia herbicola pv. millettiae
Erwinia mallotivora
Erwinia nigrifluens
Erwinia nimipressuralis
Erwinia papayae
Erwinia proteamaculans
Erwinia persicina
Enterobacter pyrinus
Erwinia psidii
Erwinia pyrifoliae
Erwinia rhapontici
Erwinia rubrifaciens
Erwinia salicis
Erwinia stewartii
Erwinia tracheiphila
Erwinia uredovora
Ewingella
Ewingella americana
Gluconobacter Asai
Gluconobacter oxydans
Herbaspirillum
Herbaspirillum rubrisubalbicans
Janthinobacterium
Janthinobacterium agaricidamnosum
Leifsonia
Leifsonia cynodontis
Leifsonia xyli
Leifsonia xyli subsp. cynodontis
Leifsonia xyli subsp. xyli
Nocardia
Nocardia vaccinii
Pantoea
Pantoea agglomerans
Pantoea agglomerans pv. gypsophilae
Pantoea agglomerans pv. millettiae
Pantoea ananatis
Pantoea ananatis pv. ananatis
Pantoea ananatis pv. uredovora
Pantoea stewartii
Pantoea stewartii subsp. indologenes
Pantoea stewartii subsp. stewartii
Pectobacterium
Pectobacterium
Pectobacterium
Pectobacterium cacticida corrig
Pectobacterium
Pectobacterium carotovorum
Pectobacterium carotovorum subsp. atrosepticum
Pectobacterium carotovorum subsp. betavasculorum
Pectobacterium carotovorum subsp. brasiliensis
Pectobacterium carotovorum subsp. carotovorum
Pectobacterium carotovorum subsp. odoriferum
Pectobacterium carotovorum subsp. wasabiae
Pectobacterium chrysanthemi
Pectobacterium chrysanthemi pv. chrysanthemi
Pectobacterium chrysanthemi pv. dianthicola
Pectobacterium chrysanthemi pv. dieffenbachiae
Pectobacterium chrysanthemi pv. parthenii
Pectobacterium chrysanthemi pv. zeae
Pectobacterium cypripedii
Pectobacterium rhapontici
Pectobacterium wasabiae
Pseudomonas
Pseudomonas agarici
Pseudomonas amygdali
Pseudomonas andropogonis pv. andropogonis
Pseudomonas andropogonis pv. sojae
Pseudomonas andropogonis pv. stizolobii
Pseudomonas asplenii
Pseudomonas avellanae
Pseudomonas avenae
Pseudomonas avenae subsp. avenae
Pseudomonas avenae subsp. citrulli
Pseudomonas avenae subsp. konjaci
Pseudomonas beteli corrig.
Pseudomonas cannabina
Pseudomonas caricapapayae
Pseudomonas caryophylli
Pseudomonas cattleyae
Pseudomonas cepacia
Pseudomonas cichorii
Pseudomonas cissicola
Pseudomonas coronafaciens
Pseudomonas corrugata
Pseudomonas costantinii
Pseudomonas dodoneae
Pseudomonas ficuserectae
Pseudomonas flectens
Pseudomonas fuscovaginae
Pseudomonas gingeri
Pseudomonas gladioli
Pseudomonas gladioli pv. agaricicola
Pseudomonas gladioli pv. alliicola
Pseudomonas gladioli pv. gladioli
Pseudomonas glumae
Pseudomonas hibiscicola
Pseudomonas marginalis
Pseudomonas marginalis pv. alfalfae
Pseudomonas marginalis pv. marginalis
Pseudomonas marginalis pv. pastinacae
Pseudomonas mediterranea
Pseudomonas meliae
Pseudomonas palleroniana
Pseudomonas plantarii
Pseudomonas pomi
Pseudomonas pseudoalcaligenes subsp. citrulli
Pseudomonas pseudoalcaligenes subsp. konjaci
Pseudomonas rubrilineans
Pseudomonas rubrisubalbicans
Pseudomonas salomonii
Pseudomonas savastanoi
Pseudomonas savastanoi pv. fraxini
Pseudomonas savastanoi pv. glycinea
Pseudomonas savastanoi pv. nerii
Pseudomonas savastanoi pv. phaseolicola
Pseudomonas savastanoi pv. retacarpa
Pseudomonas savastanoi pv. savastanoi
Pseudomonas syringae
Pseudomonas syringae pv. aceris
Pseudomonas syringae pv. actinidiae
Pseudomonas syringae pv. aesculi
Pseudomonas syringae pv. alisalensis
Pseudomonas syringae pv. antirrhini
Pseudomonas syringae pv. apii
Pseudomonas syringae pv. aptata
Pseudomonas syringae pv.
Pseudomonas syringae pv. atropurpurea
Pseudomonas syringae pv. avellanae
Pseudomonas syringae pv. avii
Pseudomonas syringae pv. berberidis
Pseudomonas syringae pv. broussonetiae
Pseudomonas syringae pv. castaneae
Pseudomonas syringae pv. cerasicola
Pseudomonas syringae pv. ciccaronei
Pseudomonas syringae pv. coriandricola
Pseudomonas syringae pv. coronafaciens
Pseudomonas syringae pv. coryli
Pseudomonas syringae pv. cunninghamiae
Pseudomonas syringae pv. daphniphylli
Pseudomonas syringae pv. delphinii
Pseudomonas syringae pv. dendropanacis
Pseudomonas syringae pv. dysoxyli
Pseudomonas syringae pv. eriobotryae
Pseudomonas syringae pv. garcae
Pseudomonas syringae pv. glycinea
Pseudomonas syringae pv. helianthi
Pseudomonas syringae pv.
Pseudomonas syringae pv.
Pseudomonas syringae pv.
Pseudomonas syringae pv. lapsa
Pseudomonas syringae pv. maculicola
Pseudomonas syringae pv.
Pseudomonas syringae pv. mori
Pseudomonas syringae pv. morsprunorum.
Pseudomonas syringae pv. myricae
Pseudomonas syringae pv.
Pseudomonas syringae pv. papulans
Pseudomonas syringae pv. passiflorae
Pseudomonas syringae pv.
Pseudomonas syringae pv. philadelphi
Pseudomonas syringae pv. photiniae
Pseudomonas syringae pv. pisi
Pseudomonas syringae pv. porri
Pseudomonas syringae pv. primulae
Pseudomonas syringae pv. rhaphiolepidis
Pseudomonas syringae pv. ribicola
Pseudomonas syringae pv. sesami
Pseudomonas syringae pv. solidagae
Pseudomonas syringae pv. spinaceae
Pseudomonas syringae pv. syringae
Pseudomonas syringae pv. tagetis
Pseudomonas syringae pv. theae
Pseudomonas syringae pv. tomato
Pseudomonas syringae pv. ulmi
Pseudomonas syringae pv. viburni
Pseudomonas syringae pv.
Pseudomonas syzygii
Pseudomonas tolaasii
Pseudomonas tremae
Pseudomonas viridiflava
Ralstonia
Ralstonia solanacearum
Ralstonia syzygii
Rathayibacter
Rathayibacter iranicus
Rathayibacter rathayi
Rathayibacter
Rathayibacter tritici
Rhizobacter
Rhizobacter dauci corrig.
Rhizobium
Rhizobium larrymoorei
Rhizobium radiobacter
Rhizobium rhizogenes
Rhizobium rubi
Rhizobium vitis
Rhodococcus
Rhodococcus fascians
Samsonia
Samsonia erythrinae
Serratia
Serratia marcescens
Serratia proteamaculans
Sphingomonas
Sphingomonas melonis Buonaurio
Sphingomonas suberifaciens
Spiroplasma
Spiroplasma citri
Spiroplasma kunkelii
Spiroplasma phoeniceum
Streptomyces
Streptomyces acidiscabies
Streptomyces albidoflavus
Streptomyces candidus
Streptomyces caviscabies
Streptomyces collinus
Streptomyces europaeiscabiei
Streptomyces intermedius
Streptomyces ipomoeae
Streptomyces luridiscabiei
Streptomyces niveiscabiei
Streptomyces puniciscabiei
Streptomyces reticuliscabei
Streptomyces scabiei corrig.
Streptomyces setonii
Streptomyces steliiscabiei
Streptomyces turgidiscabies
Streptomyces wedmorensis
Xanthomonas
Xanthomonas albilineans
Xanthomonas alfalfae
Xanthomonas alfalfae subsp. alfalfae
Xanthomonas alfalfae subsp. citrumelonis
Xanthomonas arboricola
Xanthomonas axonopodis
Xanthomonas bromi
Xanthomonas campestris
Xanthomonas cassavae
Xanthomonas citri
Xanthomonas cucurbitae
Xanthomonas euvesicatoria
Xanthomonas fragariae
Xanthomonas fuscans
Xanthomonas fuscans
Xanthomonas gardneri
Xanthomonas hortorum
Xanthomonas hortorum
Xanthomonas hyacinthi
Xanthomonas oryzae
Xanthomonas populi
Xanthomonas sacchari
Xanthomonas theicola
Xanthomonas translucens
Xanthomonas vasicola
Xylella
Xylella fastidiosa
Xylophilus
Xylophilus ampelinus
‘Candidatus’ Plant Pathogenic Bacteria
‘Candidatus Liberibacter’
‘Candidatus Liberibacter asiaticus’
‘Candidatus Phlomobacter’
‘Candidatus Phlomobacter fragariae’
‘Candidatus Phytoplasma’

TABLE 2
Further Example Bacteria
Optionally, the carrier cells are selected from this Table and/or the target cells are selected from this Table (eg,
wherein the carrier and target cells are of a different species; or of the same species but are a different strain or vice versa).

| | | | | |
|---|---|---|---|---|
| Abiotrophia | Acidocella | Actinomyces | Alkalilimnicola | Aquaspirillum |
| Abiotrophia defectiva | Acidocella aminolytica | Actinomyces bovis | Alkalilimnicola ehrlichii | Aquaspirillum polymorphum |
| Acaricomes | Acidocella facilis | Actinomyces denticolens | Alkaliphilus | Aquaspirillum putridiconchylium |
| Acaricomes phytoseiuli | Acidomonas | Actinomyces europaeus | Alkaliphilus oremlandii | Aquaspirillum serpens |
| Acetitomaculum | Acidomonas methanolica | Actinomyces georgiae | Alkaliphilus transvaalensis | Aquimarina |
| Acetitomaculum ruminis | Acidothermus | Actinomyces gerencseriae | Allochromatium | Aquimarina latercula |
| Acetivibrio | Acidothermus cellulolyticus | Actinomyces hordeovulneris | Allochromatium vinosum | Arcanobacterium |
| Acetivibrio cellulolyticus | Acidovorax | Actinomyces howellii | Alloiococcus | Arcanobacterium haemolyticum |
| Acetivibrio ethanolgignens | Acidovorax anthurii | Actinomyces hyovaginalis | Alloiococcus otitis | Arcanobacterium pyogenes |
| Acetivibrio multivorans | Acidovorax caeni | Actinomyces israelii | Allokutzneria | Archangium |
| Acetoanaerobium | Acidovorax cattleyae | Actinomyces johnsonii | Allokutzneria albata | Archangium gephyra |
| Acetoanaerobium noterae | Acidovorax citrulli | Actinomyces meyeri | Altererythrobacter | Arcobacter |
| Acetobacter | Acidovorax defluvii | Actinomyces naeslundii | Altererythrobacter ishigakiensis | Arcobacter butzleri |
| Acetobacter aceti | Acidovorax delafieldii | Actinomyces neuii | Altermonas | Arcobacter cryaerophilus |
| Acetobacter cerevisiae | Acidovorax facilis | Actinomyces odontolyticus | Altermonas haloplanktis | Arcobacter halophilus |
| Acetobacter cibinongensis | Acidovorax konjaci | Actinomyces oris | Altermonas macleodii | Arcobacter nitrofigilis |
| Acetobacter estunensis | Acidovorax temperans | Actinomyces radingae | Alysiella | Arcobacter skirrowii |
| Acetobacter fabarum | Acidovorax valerianellae | Actinomyces slackii | Alysiella crassa | Arhodomonas |
| Acetobacter ghanensis | Acinetobacter | Actinomyces turicensis | Alysiella filiformis | Arhodomonas aquaeolei |
| Acetobacter indonesiensis | Acinetobacter baumannii | Actinomyces viscosus | Aminobacter | Arsenophonus |
| Acetobacter lovaniensis | Acinetobacter baylyi | Actinoplanes | Aminobacter aganoensis | Arsenophonus nasoniae |
| Acetobacter malorum | Acinetobacter bouvetii | Actinoplanes auranticolor | Aminobacter aminovorans | Arthrobacter |
| Acetobacter nitrogenifigens | Acinetobacter calcoaceticus | Actinoplanes brasiliensis | Aminobacter niigataensis | Arthrobacter agilis |
| Acetobacter oeni | Acinetobacter gerneri | Actinoplanes consettensis | Aminobacterium | Arthrobacter albus |
| Acetobacter orientalis | Acinetobacter haemolyticus | Actinoplanes deccanensis | Aminobacterium mobile | Arthrobacter aurescens |
| Acetobacter orleanensis | Acinetobacter johnsonii | Actinoplanes derwentensis | Aminomonas | Arthrobacter chlorophenolicus |
| Acetobacter pasteurianus | Acinetobacter junii | Actinoplanes digitatis | Aminomonas paucivorans | Arthrobacter citreus |
| Acetobacter pornorurn | Acinetobacter lwoffi | Actinoplanes durhamensis | Ammoniphilus | Arthrobacter crystallopoietes |
| Acetobacter senegalensis | Acinetobacter parvus | Actinoplanes ferrugineus | Ammoniphilus oxalaticus | Arthrobacter cumminsii |
| Acetobacter xylinus | Acinetobacter radioresistens | Actinoplanes globisporus | Ammoniphilus oxalivorans | Arthrobacter globiformis |
| Acetobacterium | Acinetobacter schindleri | Actinoplanes humidus | Amphibacillus | Arthrobacter histidinolovorans |
| Acetobacterium bakii | Acinetobacter soli | Actinoplanes italicus | Amphibacillus xylanus | Arthrobacter ilicis |
| Acetobacterium carbinolicum | Acinetobacter tandoii | Actinoplanes liguriensis | Amphritea | Arthrobacter luteus |
| Acetobacterium dehalogenans | Acinetobacter tjernbergiae | Actinoplanes lobatus | Amphritea balenae | Arthrobacter methylotrophus |
| Acetobacterium fimetarium | Acinetobacter towneri | Actinoplanes missouriensis | Amphritea japonica | Arthrobacter mysorens |
| Acetobacterium malicum | Acinetobacter ursingii | Actinoplanes palleronii | Amycolatopsis | Arthrobacter nicotianae |
| Acetobacterium paludosum | Acinetobacter venetianus | Actinoplanes philippinensis | Amycolatopsis alba | Arthrobacter nicotinovorans |
| Acetobacterium tundrae | Acrocarpospora | Actinoplanes rectilineatus | Amycolatopsis albidoflavus | Arthrobacter oxydans |
| Acetobacterium wieringae | Acrocarpospora corrugata | Actinoplanes regularis | Amycolatopsis azurea | Arthrobacter pascens |
| Acetobacterium woodii | Acrocarpospora macrocephala | Actinoplanes teichomyceticus | Amycolatopsis coloradensis | Arthrobacter phenanthrenivorans |
| Acetofilamentum | Acrocarpospora pleiomorpha | Actinoplanes utahensis | Amycolatopsis lurida | Arthrobacter polychromogenes |
| Acetofilamentum rigidum | Actibacter | Actinopolyspora | Amycolatopsis mediterranei | Atrhrobacter protophormiae |
| Acetohalobium | Actibacter sediminis | Actinopolyspora halophila | Amycolatopsis rifamycinica | Arthrobacter psychrolactophilus |
| Acetohalobium arabaticum | Actinoalloteichus | Actinopolyspora mortivallis | Amycolatopsis rubida | Arthrobacter ramosus |
| Acetomicrobium | Actinoalloteichus cyanogriseus | Actinosynnema | Amycolatopsis sulphurea | Arthrobacter sulfonivorans |
| Acetomicrobium faecale | Actinoalloteichus hymeniacidonis | Actinosynnema mirum | Amycolatopsis tolypomycina | Arthrobacter sulfureus |
| Acetomicrobium flavidum | Actinoalloteichus spitiensis | Actinotalea | Anabaena | Arthrobacter uratoxydans |
| Acetonema | Actinobaccillus | Actinotalea fermentans | Anabaena cylindrica | Arthrobacter ureafaciens |
| Acetonema longum | Actinobacillus capsulatus | Aerococcus | Anabaena flos-aquae | Arthrobacter viscosus |
| Acetothermus | Actinobacillus delphinicola | Aerococcus sanguinicola | Anabaena variabilis | Arthrobacter woluwensis |
| Acetothermus paucivorans | Actinobacillus hominis | Aerococcus urinae | Anaeroarcus | Asaia |
| Acholeplasma | Actinobacillus indolicus | Aerococcus urinaeequi | Anaeroarcus burkinensis | Asaia bogorensis |
| Acholeplasma axanthum | Actinobacillus lignieresii | Aerococcus urinaehominis | Anaerobaculum | Asanoa |
| Acholeplasma brassicae | Actinobacillus minor | Aerococcus viridans | Anaerobaculum mobile | Asanoa ferruginea |
| Acholeplasma cavigenitalium | Actinobacillus muris | Aeromicrobium | Anaerobiospirillum | Asticcacaulis |
| Acholeplasma equifetale | Actinobacillus pleuropneumoniae | Aeromicrobium erythreum | Anaerobiospirillum succiniciproducens | Asticcacaulis biprosthecium |
| Acholeplasma granularum | Actinobacillus porcinus | Aeromonas | Anaerobiospirillum thomasii | Asticcacaulis excentricus |
| Acholeplasma hippikon | Actinobacillus rossii | Aeromonas allosaccharophila | Anaerococcus | Atopobacter |
| Acholeplasma laidlawii | Actinobacillus scotiae | Aeromonas bestiarum | Anaerococcus hydrogenalis | Atopobacter phocae |
| Acholeplasma modicum | Actinobacillus seminis | Aeromonas caviae | Anaerococcus lactolyticus | Atopobium |
| Acholeplasma morum | Actinobacillus succinogenes | Aeromonas encheleia | Anaerococcus prevotii | Atopobium fossor |
| Acholeplasma multilocale | Actinobaccillus suis | Aeromonas enteropelogenes | Anaerococcus tetradius | Atopobium minutum |
| Acholeplasma oculi | Actinobacillus ureae | Aeromonas eucrenophila | Anaerococcus vaginalis | Atopobium parvulum |
| Acholeplasma palmae | Actinobaculum | Aeromonas ichthiosmia | Anaerofustis | Atopobium rimae |
| Acholeplasma parvum | Actinobaculum massiliense | Aeromonas jandaei | Anaerofustis stercorihominis | Atopobium vaginae |
| Acholeplasma pleciae | Actinobaculum schaalii | Aeromonas media | Anaeromusa | Aureobacterium |
| Acholeplasma vituli | Actinobaculum suis | Aeromonas popoffii | Anaeromusa acidaminophila | Aureobacterium barkeri |
| Achromobacter | Actinomyces urinale | Aeromonas sobria | Anaeromyxobacter | Aurobacterium |
| Achromobacter denitrificans | Actinocatenispora | Aeromonas veronii | Anaeromyxobacter dehalogenans | Aurobacterium liquefaciens |
| Achromobacter insolitus | Actinocatenispora rupis | Agrobacterium | Anaerorhabdus | Avibacterium |
| Achromobacter piechaudii | Actinocatenispora thailandica | Agrobacterium gelatinovorum | Anaerorhabdus furcosa | Avibacterium avium |
| Achromobacter ruhlandii | Actinocatenispora sera | Agrococcus | Anaerosinus | Avibacterium gallinarum |
| Achromobacter spanius | Actinocorallia | Agrococcus citreus | Anaerosinus glycerini | Avibacterium paragallinarum |
| Acidaminobacter | Actinocorallia aurantiaca | Agrococcus jenensis | Anaerovirgula | Avibacterium volantium |
| Acidaminobacter hydrogenoformans | Actinocorallia aurea | Agromonas | Anaerovirgula multivorans | Azoarcus |
| Acidaminococcus | Actinocorallia cavernae | Agromonas oligotrophica | Ancalomicrobium | Azoarcus indigens |
| Acidaminococcus fermentans | Actinocorallia glomerata | Agromyces | Ancalomicrobium adetum | Azoarcus tolulyticus |
| Acidaminococcus intestini | Actinocorallia herbida | Agromyces fucosus | Ancylobacter | Azoarcus toluvorans |
| Acidicaldus | Actinocorallia libanotica | Agromyces hippuratus | Ancylobacter aquaticus | Azohydromonas |
| Acidicaldus organivorans | Actinocorallia longicatena | Agromyces luteolus | Aneurinibacillus | Azohydromonas australica |
| Acidimicrobium | Actinomadura | Agromyces mediolanus | Aneurinibacillus aneurinilyticus | Azohydromonas lata |
| Acidimicrobium ferrooxidans | Actinomadura alba | Agromyces ramosus | Aneurinibacillus migulanus | Azomonas |
| Acidiphilium | Actinomadura atramentaria | Agromyces rhizospherae | Aneurinibacillus thermoaerophilus | Azomonas agilis |
| Acidiphilium acidophilum | Actinomadura bangladeshensis | Akkermansia | Angiococcus | Azomonas insignis |
| Acidiphilium angustum | Actinomadura catellatispora | Akkermansia muciniphila | Angiococcus disciformis | Azomonas macrocytogenes |
| Acidiphilium cryptum | Actinomadura chibensis | Albidiferax | Angulomicrobium | Azorhizobium |
| Acidiphilium multivorum | Actinomadura chokoriensis | Albidiferax ferrireducens | Angulomicrobium tetraedrale | Azorhizobium caulinodans |
| Acidiphilium organovorum | Actinomadura citrea | Albidovulum | Anoxybacillus | Azorhizophilus |
| Acidiphilium rubrum | Actinomadura coerulea | Albidovulum inexpectatum | Anoxybacillus pushchinoensis | Azorhizophilus paspali |
| Acidisoma | Actinomadura echinospora | Alcaligenes | Aquabacterium | Azospirillum |
| Acidisoma sibiricum | Actinomadura fibrosa | Alcaligenes denitrificans | Aquabacterium commune | Azospirillum brasilense |
| Acidisoma tundrae | Actinomadura formosensis | Alcaligenes faecalis | Aquabacterium parvum | Azospirillum halopraeferens |
| Acidisphaera | Actinomadura hibisca | Alcanivorax | Borrelia | Azospirillum irakense |
| Acidisphaera rubrifaciens | Actinomadura kijaniata | Alcanivorax borkumensis | Borrelia afzelii | Azotobacter |
| Acidithiobacillus | Actinomadura latina | Alcanivorax jadensis | Borrelia americana | Azotobacter beijerinckii |
| Acidithiobacillus albertensis | Actinomadura livida | Algicola | Borrelia burgdorferi | Azotobacter chroococcum |
| Acidithiobacillus caldus | Actinomadura luteofluorescens | Algicola bacteriolytica | Borrelia carolinensis | Azotobacter nigricans |
| Acidithiobacillus ferrooxidans | Actinomadura macra | Alicyclobacillus | Borrelia coriaceae | Azotobacter salinestris |
| Acidithiobacillus thiooxidans | Actinomadura madurae | Alicyclobacillus disulfidooxidans | Borrelia garinii | Azotobacter vinelandii |
| Acidobacterium | Actinomadura oligospora | Alicyclobacillus sendaiensis | Borrelia japonica | Brevinema |
| Acidobacterium capsulatum | Actinomadura pelletieri | Alicyclobacillus vulcanalis | Bosea | Brevinema andersonii |
| Bacillus | Actinomadura rubrobrunea | Alishewanella | Bosea minatitlanensis | Brevundimonas |
| [see below] | Actinomadura rugatobispora | Alishewanella fetalis | Bosea thiooxidans | Brevundimonas alba |
| Bacteriovorax | Actinomadura umbrina | Alkalibacillus | Brachybacterium | Brevundimonas aurantiaca |
| Bacteriovorax stolpii | Actinomadura verrucosospora | Alkalibacillus haloalkaliphilus | Brachybacterium alimentarium | Brevundimonas diminuta |
| Bacillus | Actinomadura vinacea | Bibersteinia | Brachybacterium faecium | Brevundimonas intermedia |
| B. acidiceler | Actinomadura viridilutea | Bibersteinia trehalosi | Brachybacterium paraconglomeratum | Brevundimonas subvibrioides |
| B. acidicola | Actinomadura viridis | Bifidobacterium | Brachybacterium rhamnosum | Brevundimonas vancanneytii |
| B. acidiproducens | Actinomadura yumaensis | Bifidobacterium adolescentis | Brachybacterium tyrofermentans | Brevundimonas variabilis |
| B. acidocaldarius | Bacteroides | Bifidobacterium angulatum | Brachyspira | Brevundimonas vesicularis |
| B. acidoterrestris | Bacteroides caccae | Bifidobacterium animalis | Brachyspira alvinipulli | Brochothrix |
| B. aeolius | Bacteroides coagulans | Bifidobacterium asteroides | Brachyspira hyodysenteriae | Brochothrix campestris |
| B. aerius | Bacteroides eggerthii | Bifidobacterium bifidum | Brachyspira innocens | Brochothrix thermosphacta |
| B. aerophilus | Bacteroides fragilis | Bifidobacterium boum | Brachyspira murdochii | Brucella |
| B. agaradhaerens | Bacteroides galacturonicus | Bifidobacterium breve | Brachyspira pilosicoli | Brucella canis |
| B. agri | Bacteroides helcogenes | Bifidobacterium catenulatum | Bradyrhizobium | Brucella neotomae |
| B. aidingensis | Bacteroides ovatus | Bifidobacterium choerinum | Bradyrhizobium canariense | Bryobacter |
| B. akibai | Bacteroides pectinophilus | Bifidobacterium coryneforme | Bradyrhizobium elkanii | Bryobacter aggregatus |
| B. alcalophilus | Bacteroides pyogenes | Bifidobacterium cuniculi | Bradyrhizobium japonicum | Burkholderia |
| B. algicola | Bacteroides salyersiae | Bifidobacterium dentium | Bradyrhizobium liaoningense | Burkholderia ambifaria |
| B. alginolyticus | Bacteroides stercoris | Bifidobacterium gallicum | Brenneria | Burkholderia andropogonis |
| B. alkalidiazotrophicus | Bacteroides suis | Bifidobacterium gallinarum | Brenneria alni | Burkholderia anthina |
| B. alkalinitrilicus | Bacteroides tectus | Bifidobacterium indicum | Brenneria nigrifluens | Burkholderia caledonica |
| B. alkalisediminis | Bacteroides thetaiotaomicron | Bifidobacterium longum | Brenneria quercina | Burkholderia caryophylli |
| B. alkalitelluris | Bacteroides uniformis | Bifidobacterium | Brenneria quercina | Burkholderia cenocepacia |
| B. altitudinis | Bacteroides ureolyticus | magnumBifidobacterium | Brenneria salicis | Burkholderia cepacia |
| B. alveayuensis | Bacteroides vulgatus | merycicum | Brevibacillus | Burkholderia cocovenenans |
| B. alvei | Balnearium | Bifidobacterium minimum | Brevibacillus agri | Burkholderia dolosa |
| B. amyloliquefaciens | Balnearium lithotrophicum | Bifidobacterium pseudocatenulatum | Brevibacillus borstelensis | Burkholderia fungorum |
| B. a. subsp. amyloliquefaciens | Balneatrix | Bifidobacterium pseudolongum | Brevibacillus brevis | Burkholderia glathei |
| B. a. subsp. plantarum | Balneatrix alpica | Bifidobacterium pullorum | Brevibacillus centrosporus | Burkholderia glumae |
| B. dipsosauri | Balneola | Bifidobacterium ruminantium | Brevibacillus choshinensis | Burkholderia graminis |
| B. drentensis | Balneola vulgaris | Bifidobacterium saeculare | Brevibacillus invocatus | Burkholderia kururiensis |
| B. edaphicus | Barnesiella | Bifidobacterium subtile | Brevibacillus laterosporus | Burkholderia multivorans |
| B. ehimensis | Barnesiella viscericola | Bifidobacterium thermophilum | Brevibacillus parabrevis | Burkholderia phenazinium |
| B. eiseniae | Bartonella | Bilophila | Brevibacillus reuszeri | Burkholderia plantarii |
| B. enclensis | Bartonella alsatica | Bilophila wadsworthia | Brevibacterium | Burkholderia pyrrocinia |
| B. endophyticus | Bartonella bacilliformis | Biostraticola | Brevibacterium abidum | Burkholderia silvatlantica |
| B. endoradicis | Bartonella clarridgeiae | Biostraticola tofi | Brevibacterium album | Burkholderia stabilis |
| B. farraginis | Bartonella doshiae | Bizionia | Brevibacterium aurantiacum | Burkholderia thailandensis |
| B. fastidiosus | Bartonella elizabethae | Bizionia argentinensis | Brevibacterium celere | Burkholderia tropica |
| B. fengqiuensis | Bartonella grahamii | Blastobacter | Brevibacterium epidermidis | Burkholderia unamae |
| B. firmus | Bartonella henselae | Blastobacter capsulatus | Brevibacterium frigoriTolerans | Burkholderia vietnamiensis |
| B. flexus | Bartonella rochalimae | Blastobacter denitrificans | Brevibacterium halotolerans | Buttiauxella |
| B. foraminis | Bartonella vinsonii | Blastococcus | Brevibacterium iodinum | Buttiauxella agrestis |
| B. fordii | Bavariicoccus | Blastococcus aggregatus | Brevibacterium linens | Buttiauxella brennerae |
| B. formosus | Bavariicoccus seileri | Blastococcus saxobsidens | Brevibacterium lyticum | Buttiauxella ferragutiae |
| B. fortis | Bdellovibrio | Blastochloris | Brevibacterium mcbrellneri | Buttiauxella gaviniae |
| B. fumarioli | Bdellovibrio bacteriovorus | Blastochloris viridis | Brevibacterium otitidis | Buttiauxella izardii |
| B. funiculus | Bdellovibrio exovorus | Blastomonas | Brevibacterium oxydans | Buttiauxella noackiae |
| B. fusiformis | Beggiatoa | Blastomonas natatoria | Brevibacterium paucivorans | Buttiauxella warmboldiae |
| B. galactophilus | Beggiatoa alba | Blastopirellula | Brevibacterium stationis | Butyrivibrio |
| B. galactosidilyticus | Beijerinckia | Blastopirellula marina | B. taeanensis | Butyrivibrio fibrisolvens |
| B. galliciensis | Beijerinckia derxii | Blautia | B. tequilensis | Butyrivibrio hungatei |
| B. gelatini | Beijerinckia fluminensis | Blautia coccoides | B. thermantarcticus | Butyrivibrio proteoclasticus |
| B. gibsonii | Beijerinckia indica | Blautia hansenii | B. thermoaerophilus | B. lautus |
| B. ginsengi | Beijerinckia mobilis | Blautia producta | B. thermoamylovorans | B. lehensis |
| B. ginsengihumi | Belliella | Blautia wexlerae | B. thermocatenulatus | B. lentimorbus |
| B. ginsengisoli | Belliella baltica | Bogoriella | B. thermocloacae | B. lentus |
| B. globisporus | Bellilinea | Bogoriella caseilytica | B. thermocopriae | B. licheniformis |
| (eg, B. g. subsp. Globisporus; | Bellilinea caldifistulae | Bordetella | B. thermodenitrificans | B. ligniniphilus |
| or B. g. subsp. Marinus) | Belnapia | Bordetella avium | B. thermoglucosidasius | B. litoralis |
| Caenimonas | Belnapia moabensis | Bordetella bronchiseptica | B. thermolactis | B. locisalis |
| Caenimonas koreensis | Bergeriella | Bordetella hinzii | B. thermoleovorans | B. luciferensis |
| Caldalkalibacillus | Bergeriella denitrificans | Bordetella holmesii | B. thermophilus | B. luteolus |
| Caldalkalibacillus uzonensis | Beutenbergia | Bordetella parapertussis | B. thermoruber | B. luteus |
| Caldanaerobacter | Beutenbergia cavernae | Bordetella pertussis | B. thermosphaericus | B. macauensis |
| Caldanaerobacter subterraneus | B. aminovorans | Bordetella petrii | B. thiaminolyticus | B. macerans |
| Caldanaerobius | B. amylolyticus | Bordetella trematum | B. thioparans | B. macquariensis |
| Caldanaerobius fijiensis | B. andreesenii | B. glucanolyticus | B. thuringiensis | B. macyae |
| Caldanaerobius polysaccharolyticus | B. aneurinilyticus | B. gordonae | B. tianshenii | B. malacitensis |
| Caldanaerobius zeae | B. anthracis | B. gottheilii | B. trypoxylicola | B. mannanilyticus |
| Caldanaerovirga | B. aquimaris | B. graminis | B. tusciae | B. marisflavi |
| Caldanaerovirga acetigignens | B. arenosi | B. halmapalus | B. validus | B. marismortui |
| Caldicellulosiruptor | B. arseniciselenatis | B. haloalkaliphilus | B. vallismortis | B. marmarensis |
| Caldicellulosiruptor bescii | B. arsenicus | B. halochares | B. vedderi | B. massiliensis |
| Caldicellulosiruptor kristjanssonii | B. aurantiacus | B. halodenitrificans | B. velezensis | B. megaterium |
| Caldicellulosiruptor owensensis | B. arvi | B. halodurans | B. vietnamensis | B. mesonae |
| | B. aryabhattai | B. halophilus | B. vireti | B. methanolicus |
| | B. asahii | B. halosaccharovorans | B. vulcani | B. methylotrophicus |
| | B. atrophaeus | B. hemicellulosilyticus | B. wakoensis | B. migulanus |
| | B. axarquiensis | B. hemicentroti | B. weihenstephanensis | B. mojavensis |
| | B. azotofixans | B. herbersteinensis | B. xiamenensis | B. mucilaginosus |
| | B. azotoformans | B. horikoshii | B. xiaoxiensis | B. muralis |
| | B. badius | B. horneckiae | B. zhanjiangensis | B. murimartini |
| | B. barbaricus | B. horti | B. peoriae | B. mycoides |
| | B. bataviensis | B. huizhouensis | B. persepolensis | B. naganoensis |
| | B. beijingensis | B. humi | B. persicus | B. nanhaiensis |
| | B. benzoevorans | B. hwajinpoensis | B. pervagus | B. nanhaiisediminis |
| | B. beringensis | B. idriensis | B. plakortidis | B. nealsonii |
| | B. berkeleyi | B. indicus | B. pocheonensis | B. neidei |
| | B. beveridgei | B. infantis | B. polygoni | B. neizhouensis |
| | B. bogoriensis | B. infernus | B. polymyxa | B. niabensis |
| | B. boroniphilus | B. insolitus | B. popilliae | B. niacini |
| | B. borstelensis | B. invictae | B. pseudalcalophilus | B. novalis |
| | B. brevis Migula | B. iranensis | B. pseudofirmus | B. oceanisediminis |
| | B. butanolivorans | B. isabeliae | B. pseudomycoides | B. odysseyi |
| | B. canaveralius | B. isronensis | B. psychrodurans | B. okhensis |
| | B. carboniphilus | B. jeotgali | B. psychrophilus | B. okuhidensis |
| | B. cecembensis | B. kaustophilus | B. psychrosaccharolyticus | B. oleronius |
| | B. cellulosilyticus | B. kobensis | B. psychrotolerans | B. oryzaecorticis |
| | B. centrosporus | B. kochii | B. pulvifaciens | B. oshimensis |
| | B. cereus | B. kokeshiiformis | B. pumilus | B. pabuli |
| | B. chagannorensis | B. koreensis | B. purgationiresistens | B. pakistanensis |
| | B. chitinolyticus | B. korlensis | B. pycnus | B. pallidus |
| | B. chondroitinus | B. kribbensis | B. qingdaonensis | B. pallidus |
| | B. choshinensis | B. krulwichiae | B. qingshengii | B. panacisoli |
| | B. chungangensis | B. laevolacticus | B. reuszeri | B. panaciterrae |
| | B. cibi | B. larvae | B. rhizosphaerae | B. pantothenticus |
| | B. circulans | B. laterosporus | B. rigui | B. parabrevis |
| | B. clarkii | B. salexigens | B. ruris | B. paraflexus |
| | B. clausii | B. saliphilus | B. safensis | B. pasteurii |
| | B. coagulans | B. schlegelii | B. salarius | B. patagoniensis |
| | B. coahuilensis | B. sediminis | Catenuloplanes | Curtobacterium |
| | B. cohnii | B. selenatarsenatis | Catenuloplanes atrovinosus | Curtobacterium albidum |
| | B. composti | B. selenitireducens | Catenuloplanes castaneus | Curtobacterium citreus |
| | B. curdlanolyticus | B. seohaeanensis | Catenuloplanes crispus | |
| | B. cycloheptanicus | B. shacheensis | Catenuloplanes indicus | |
| | B. cytotoxicus | B. shackletonii | Catenuloplanes japonicus | |
| | B. daliensis | B. siamensis | Catenuloplanes nepalensis | |
| | B. decisifrondis | B. silvestris | Catenuloplanes niger | |
| | B. decolorationis | B. simplex | Chryseobacterium | |
| | B. deserti | B. siralis | Chryseobacterium balustinum | |
| | Campylobacter | B. smithii | Citrobacter | |
| | Campylobacter coli | B. soli | C. amalonaticus | |
| | Campylobacter concisus | B. solimangrovi | C. braakii | |
| | Campylobacter curvus | B. solisalsi | C. diversus | |
| | Campylobacter fetus | B. songklensis | C. farmeri | |
| | Campylobacter gracilis | B. sonorensis | C. freundii | |
| | Campylobacter helveticus | B. sphaericus | C. gillenii | |
| | Campylobacter hominis | B. sporothermodurans | C. koseri | |
| | Campylobacter hyointestinalis | B. stearothermophilus | C. murliniae | |
| | Campylobacter jejuni | B. stratosphericus | C. pasteurii | |
| | Campylobacter lari | B. subterraneus | C. rodentium | |
| | Campylobacter mucosalis | B. subtilis | C. sedlakii | |
| | Campylobacter rectus | (eg, B. s. subsp. Inaquosorum; | C. werkmanii | |
| | Campylobacter showae | or B. s. subsp. Spizizeni; | C. youngae | |
| | Campylobacter sputorum | or B. s. subsp. Subtilis) | Clostridium | |
| | Campylobacter upsaliensis | Cardiobacterium | (see below) | |
| | Capnocytophaga | Cardiobacterium hominis | Coccochloris | |
| | Capnocytophaga canimorsus | Carnimonas | Coccochloris elabens | |
| | Capnocytophaga cynodegmi | Carnimonas nigrificans | Corynebacterium | |
| | Capnocytophaga gingivalis | Carnobacterium | Corynebacterium flavescens | |
| | Capnocytophaga granulosa | Carnobacterium alterfunditum | Corynebacterium variabile | |
| | Capnocytophaga haemolytica | Carnobacterium divergens | | |
| | Capnocytophaga ochracea | Carnobacterium funditum | | |
| | Capnocytophaga sputigena | Carnobacterium gallinarum | | |
| | | Carnobacterium maltaromaticum | | |
| | | Carnobacterium mobile | | |
| | | Carnobacterium viridans | | |
| | | Caryophanon | | |
| | | Caryophanon latum | | |
| | | Caryophanon tenue | | |
| | | Catellatospora | | |
| | | Catellatospora citrea | | |
| | | Catellatospora methionotrophica | | |
| | | Catenococcus | | |
| | | Catenococcus thiocycli | | |
| Clostridium | | | | |

Clostridium absonum, Clostridium aceticum, Clostridium acetireducens, Clostridium acetobutylicum, Clostridium acidisoli, Clostridium aciditolerans,
Clostridium acidurici, Clostridium aerotolerans, Clostridium aestuarii, Clostridium akagii, Clostridium aldenense, Clostridium aldrichii, Clostridium
algidicarni, Clostridium algidixylanolyticum, Clostridium algifaecis, Clostridium algoriphilum, Clostridium alkalicellulosi, Clostridium aminophilum,
Clostridium aminovalericum, Clostridium amygdalinum, Clostridium amylolyticum, Clostridium arbusti, Clostridium arcticum, Clostridium argentinense,
Clostridium asparagiforme, Clostridium aurantibutyricum, Clostridium autoethanogenum, Clostridium baratii, Clostridium barkeri, Clostridium bartlettii,
Clostridium beijerinckii, Clostridium bifermentans, Clostridium bolteae, Clostridium bornimense, Clostridium botulinum, Clostridium bowmanii, Clostridium
bryantii, Clostridium butyricum, Clostridium cadaveris, Clostridium caenicola, Clostridium caminithermale, Clostridium carboxidivorans, Clostridium carnis,
Clostridium cavendishii, Clostridium celatum, Clostridium celerecrescens, Clostridium cellobioparum, Clostridium cellulofermentans, Clostridium
cellulolyticum, Clostridium cellulosi, Clostridium cellulovorans, Clostridium chartatabidum, Clostridium chauvoei, Clostridium chromiireducens, Clostridium
citroniae, Clostridium clariflavum, Clostridium clostridioforme, Clostridium coccoides, Clostridium cochlearium, Clostridium colletant, Clostridium colicanis,
Clostridium colinum, Clostridium collagenovorans, Clostridium cylindrosporum, Clostridium difficile, Clostridium diolis, Clostridium disporicum,
Clostridium drakei, Clostridium durum, Clostridium estertheticum, Clostridium estertheticum estertheticum, Clostridium estertheticum laramiense,
Clostridium fallax, Clostridium felsineum, Clostridium fervidum, Clostridium fimetarium, Clostridium formicaceticum, Clostridium frigidicarnis, Clostridium
frigoris, Clostridium ganghwense, Clostridium gasigenes, Clostridium ghonii, Clostridium glycolicum, Clostridium glycyrrhizinilyticum, Clostridium grantii,
Clostridium haemolyticum, Clostridium halophilum, Clostridium hastiforme, Clostridium hathewayi, Clostridium herbivorans, Clostridium hiranonis,
Clostridium histolyticum, Clostridium homopropionicum, Clostridium huakuii, Clostridium hungatei, Clostridium hydrogeniformans, Clostridium
hydroxybenzoicum, Clostridium hylemonae, Clostridium jejuense, Clostridium indolis, Clostridium innocuum, Clostridium intestinale, Clostridium irregulare,
Clostridium isatidis, Clostridium josui, Clostridium kluyveri, Clostridium lactatifermentans, Clostridium lacusfryxellense, Clostridium laramiense, Clostridium
lavalense, Clostridium lentocellum, Clostridium lentoputrescens, Clostridium leptum, Clostridium limosum, Clostridium litorale, Clostridium lituseburense,
Clostridium ljungdahlii, Clostridium lortetii, Clostridium lundense, Clostridium magnum, Clostridium malenominatum, Clostridium mangenotii, Clostridium
mayombei, Clostridium methoxybenzovorans, Clostridium methylpentosum, Clostridium neopropionicum, Clostridium nexile, Clostridium nitrophenolicum,
Clostridium novyi, Clostridium oceanicum, Clostridium orbiscindens, Clostridium oroticum, Clostridium oxalicum, Clostridium papyrosolvens, Clostridium
paradoxum, Clostridium paraperfringens (Alias: C. welchii), Clostridium paraputrificum, Clostridium pascui, Clostridium pasteurianum, Clostridium
peptidivorans, Clostridium perenne, Clostridium perfringens, Clostridium pfennigii, Clostridium phytofermentans, Clostridium piliforme, Clostridium
polysaccharolyticum, Clostridium populeti, Clostridium propionicum, Clostridium proteoclasticum, Clostridium proteolyticum, Clostridium psychrophilum,
Clostridium puniceum, Clostridium purinilyticum, Clostridium putrefaciens, Clostridium putrificum, Clostridium quercicolum, Clostridium quinii, Clostridium
ramosum, Clostridium rectum, Clostridium roseum, Clostridium saccharobutylicum, Clostridium saccharogumia, Clostridium saccharolyticum, Clostridium
saccharoperbutylacetonicum, Clostridium sardiniense, Clostridium sartagoforme, Clostridium scatologenes, Clostridium schirmacherense, Clostridium
scindens, Clostridium septicum, Clostridium sordellii, Clostridium sphenoides, Clostridium spiroforme, Clostridium sporogenes, Clostridium
sporosphaeroides, Clostridium stercorarium, Clostridium stercorarium leptospartum, Clostridium stercorarium stercorarium, Clostridium stercorarium
thermolacticum, Clostridium sticklandii, Clostridium straminisolvens, Clostridium subterminale, Clostridium sufflavum, Clostridium sulfidigenes, Clostridium
symbiosum, Clostridium tagluense, Clostridium tepidiprofundi, Clostridium termitidis, Clostridium tertium, Clostridium tetani, Clostridium tetanomorphum,
Clostridium thermaceticum, Clostridium thermautotrophicum, Clostridium thermoalcaliphilum, Clostridium thermobutyricum, Clostridium thermocellum,
Clostridium thermocopriae, Clostridium thermohydrosulfuricum, Clostridium thermolacticum, Clostridium thermopalmarium, Clostridium
thermopapyrolyticum, Clostridium thermosaccharolyticum, Clostridium thermosuccinogenes, Clostridium thermosulfurigenes, Clostridium
thiosulfatireducens, Clostridium tyrobutyricum, Clostridium uliginosum, Clostridium ultunense, Clostridium villosum, Clostridium vincentii, Clostridium
viride, Clostridium xylanolyticum, Clostridium xylanovorans

| | | | | |
|---|---|---|---|---|
| Dactylosporangium | Deinococcus | Delftia | Echinicola | Nesterenkonia |
| Dactylosporangium aurantiacum | Deinococcus aerius | Delftia acidovorans | Echinicola pacifica | Nesterenkonia holobia |
| Dactylosporangium fulvum | Deinococcus apachensis | Desulfovibrio | Echinicola vietnamensis | Nocardia |
| Dactylosporangium matsuzakiense | Deinococcus aquaticus | Desulfovibrio desulfuricans | Flavobacterium | Nocardia argentinensis |
| Dactylosporangium roseum | Deinococcus aquatilis | Diplococcus | Flavobacterium antarcticum | Nocardia corallina |
| Dactylosporangium thailandense | Deinococcus caeni | Diplococcus pneumoniae | Flavobacterium aquatile | Nocardia otitidiscaviarum |
| Dactylosporangium vinaceum | Deinococcus radiodurans | Faecalibacterium | Flavobacterium aquidurense | L. sakei |
| Enterobacter | Deinococcus radiophilus | Faecalibacterium prausnitzii | Flavobacterium balustinum | L. salivarius |
| E. aerogenes | Enterobacter kobei | Fangia | Flavobacterium croceum | L. sanfranciscensis |
| E. amnigenus | E. ludwigii | Fangia hongkongensis | Flavobacterium cucumis | L. satsumensis |
| E. agglomerans | E. mori | Fastidiosipila | Flavobacterium daejeonense | L. secaliphilus |
| E. arachidis | E. nimipressuralis | Fastidiosipila sanguinis | Flavobacterium defluvii | L. sharpeae |
| E. asburiae | E. oryzae | Fusobacterium | Flavobacterium degerlachei | L. siliginis |
| E. cancerogenous | E. pulveris | Fusobacterium nucleatum | Flavobacterium denitrificans | L. spicheri |
| E. cloacae | E. pyrinus | Ideonella | Flavobacterium filum | L. suebicus |
| E. cowanii | E. radicincitans | Ideonella azotifigens | Flavobacterium flevense | L. thailandensis |
| E. dissolvens | E. taylorae | Idiomarina | Flavobacterium frigidarium | L. ultunensis |
| E. gergoviae | E. turicensis | Idiomarina abyssalis | Flavobacterium mizutaii | L. vaccinostercus |
| E. helveticus | E. sakazakii | Idiomarina baltica | Flavobacterium okeanokoites | L. vaginalis |
| E. hormaechei | Enterobacter soli | Idiomarina fontislapidosi | Janibacter | L. versmoldensis |
| E. intermedius | Enterococcus | Idiomarina loihiensis | Janibacter anophelis | L. vini |
| Gaetbulibacter | Enterococcus durans | Idiomarina ramblicola | Janibacter corallicola | L. vitulinus |
| Gaetbulibacter saemankumensis | Enterococcus faecalis | Idiomarina seosinensis | Janibacter limosus | L. zeae |
| Gallibacterium | Enterococcus faecium | Idiomarina zobellii | Janibacter melonis | L. zymae |
| Gallibacterium anatis | Erwinia | Ignatzschineria | Janibacter terrae | L. gastricus |
| Gallicola | Erwinia hapontici | Ignatzschineria larvae | Jannaschia | L. ghanensis |
| Gallicola barnesae | Escherichia | Ignavigranum | Jannaschia cystaugens | L. graminis |
| Garciella | Escherichia coli | Ignavigranum ruoffiae | Jannaschia helgolandensis | L. hammesii |
| Garciella nitratireducens | Haemophilus | Ilumatobacter | Jannaschia pohangensis | L. hamsteri |
| Geobacillus | Haemophilus aegyptius | Ilumatobacter fluminis | Jannaschia rubra | L. harbinensis |
| Geobacillus thermoglucosidasius | Haemophilus aphrophilus | Ilyobacter | Janthinobacterium | L. hayakitensis |
| Geobacillus stearothermophilus | Haemophilus felis | Ilyobacter delafieldii | Janthinobacterium agaricidamnosum | Tatlockia |
| Geobacter | Haemophilus gallinarum | Ilyobacter insuetus | Janthinobacterium lividum | Tatlockia maceachernii |
| Geobacter bemidjiensis | Haemophilus haemolyticus | Ilyobacter polytropus | Jejuia | Tatlockia micdadei |
| Geobacter bremensis | Haemophilus influenzae | Ilyobacter tartaricus | Jejuia pallidilutea | Tenacibaculum |
| Geobacter chapellei | Haemophilus paracuniculus | Listeria ivanovii | Jeotgalibacillus | Tenacibaculum amylolyticum |
| Geobacter grbiciae | Haemophilus parahaemolyticus | L. marthii | Jeotgalibacillus alimentarius | Tenacibaculum discolor |
| Geobacter hydrogenophilus | Haemophilus parainfluenzae | L. monocytogenes | Jeotgalicoccus | Tenacibaculum gallaicum |
| Geobacter lovleyi | Haemophilus paraphrohaemolyticus | L. newyorkensis | Jeotgalicoccus halotolerans | Tenacibaculum lutimaris |
| Geobacter metallireducens | Haemophilus parasuis | L. riparia | Micrococcus | Tenacibaculum mesophilum |
| Geobacter pelophilus | Haemophilus pittmaniae | L. rocourtiae | Micrococcus luteus | Tenacibaculum skagerrakense |
| Geobacter pickeringii | Hafnia | L. seeligeri | Micrococcus lylae | Tepidanaerobacter |
| Geobacter sulfurreducens | Hafnia alvei | L. weihenstephanensis | Moraxella | Tepidanaerobacter syntrophicus |
| Geodermatophilus | Hahella | L. welshimeri | Moraxella bovis | Tepidibacter |
| Geodermatophilus obscurus | Hahella ganghwensis | Listonella | Moraxella nonliquefaciens | Tepidibacter formicigenes |
| Gluconacetobacter | Halalkalibacillus | Listonella anguillarum | Moraxella osloensis | Tepidibacter thalassicus |
| Gluconacetobacter xylinus | Halalkalibacillus halophilus | Macrococcus | Nakamurella | Thermus |
| Gordonia | Helicobacter | Macrococcus bovicus | Nakamurella multipartita | Thermus aquaticus |
| Gordonia rubripertincta | Helicobacter pylori | Marinobacter | Nannocystis | Thermus filiformis |
| Kaistia | Labedella | Marinobacter algicola | Nannocystis pusilla | Thermus thermophilus |
| Kaistia adipata | Labedella gwakjiensis | Marinobacter bryozoorum | Natranaerobius | Xanthobacter |
| Kaistia soli | Labrenzia | Marinobacter flavimaris | Natranaerobius | Xanthobacter agilis |
| Kangiella | Labrenzia aggregata | Meiothermus | thermophilus | Xanthobacter aminoxidans |
| Kangiella aquimarina | Labrenzia alba | Meiothermus ruber | Natranaerobius trueperi | Xanthobacter autotrophicus |
| Kangiella koreensis | Labrenzia alexandrii | Methylophilus | Naxibacter | Xanthobacter flavus |
| Kerstersia | Labrenzia marina | Methylophilus methylotrophus | Naxibacter alkalitolerans | Xanthobacter tagetidis |
| Kerstersia gyiorum | Labrys | Microbacterium | Neisseria | Xanthobacter viscosus |
| Kiloniella | Labrys methylaminiphilus | Microbacterium ammoniaphilum | Neisseria cinerea | Xanthomonas |
| Kiloniella laminariae | Labrys miyagiensis | Microbacterium arborescens | Neisseria denitrificans | Xanthomonas albilineans |
| Klebsiella | Labrys monachus | Microbacterium liquefaciens | Neisseria gonorrhoeae | Xanthomonas alfalfae |
| K. granulomatis | Labrys okinawensis | Microbacterium oxydans | Neisseria lactamica | Xanthomonas arboricola |
| K. oxytoca | Labrys portucalensis | L. mali | Neisseria mucosa | Xanthomonas axonopodis |
| K. pneumoniae | Lactobacillus | L. manihotivorans | Neisseria sicca | Xanthomonas campestris |
| K. terrigena | [see below] | L. mindensis | Neisseria subflava | Xanthomonas citri |
| K. variicola | Laceyella | L. mucosae | Neptunomonas | Xanthomonas codiaei |
| Kluyvera | Laceyella putida | L. murinus | Neptunomonas japonica | Xanthomonas cucurbitae |
| Kluyvera ascorbata | Lechevalieria | L. nagelii | L. parakefiri | Xanthomonas euvesicatoria |
| Kocuria | Lechevalieria aerocolonigenes | L. namurensis | L. paralimentarius | Xanthomonas fragariae |
| Kocuria roasea | Legionella | L. nantensis | L. paraplantarum | Xanthomonas fuscans |
| Kocuria varians | [see below] | L. oligofermentans | L. pentosus | Xanthomonas gardneri |
| Kurthia | Listeria | L. oris | L. perolens | Xanthomonas hortorum |
| Kurthia zopfii | L. aquatica | L. panis | L. plantarum | Xanthomonas hyacinthi |
| Lactobacillus | L. booriae | L. pantheris | L. pontis | Xanthomonas perforans |
| L. acetotolerans | L. cornellensis | L. parabrevis | L. protectus | Xanthomonas phaseoli |
| L. acidifarinae | L. fleischmannii | L. parabuchneri | L. psittaci | Xanthomonas pisi |
| L. acidipiscis | L. floridensis | L. paracasei | L. rennini | Xanthomonas populi |
| L. acidophilus | L. grandensis | L. paracollinoides | L. reuteri | Xanthomonas theicola |
| Lactobacillus agilis | L. grayi | L. parafarraginis | L. rhamnosus | Xanthomonas translucens |
| L. algidus | L. innocua | L. homohiochii | L. rimae | Xanthomonas vesicatoria |
| L. alimentarius | L. catenaformis | L. iners | L. rogosae | Xylella |
| L. amylolyticus | L. ceti | L. ingluviei | L. rossiae | Xylella fastidiosa |
| L. amylophilus | L. coleohominis | L. intestinalis | L. ruminis | Xylophilus |
| L. amylotrophicus | L. collinoides | L. fuchuensis | L. saerimneri | Xylophilus ampelinus |
| L. amylovorus | L. composti | L. gallinarum | L. jensenii | Zobellella |
| L. animalis | L. concavus | L. gasseri | L. johnsonii | Zobellella denitrificans |
| L. antri | L. coryniformis | Candidatus Legionella jeonii | L. kalixensis | Zobellella taiwanensis |
| L. apodemi | L. crispatus | Legionella jordanis | L. kefiranofaciens | Zeaxanthinibacter |
| L. aviarius | L. crustorum | Legionella lansingensis | L. kefiri | Zeaxanthinibacter enoshimensis |
| L. bifermentans | L. curvatus | Legionella londiniensis | L. kimchii | Zhihengliuella |
| L. brevis | L. delbrueckii subsp. bulgaricus | Legionella longbeachae | L. helveticus | Zhihengliuella halotolerans |
| L. buchneri | L. delbrueckii subsp. delbrueckii | Legionella lytica | L. hilgardii | Xylanibacterium |
| L. camelliae | L. delbrueckii subsp. lactis | Legionella maceachernii | Legionella quinlivanii | Xylanibacterium ulmi |
| L. casei | L. dextrinicus | Legionella massiliensis | Legionella rowbothamii | |
| L. kitasatonis | L. diolivorans | Legionella micdadei | Legionella rubrilucens | |
| L. kunkeei | L. equi | Legionella monrovica | Legionella sainthelensi | |
| L. leichmannii | L. equigenerosi | Legionella moravica | Legionella santicrucis | |
| L. lindneri | L. farraginis | Legionella nagasakiensis | Legionella shakespearei | |
| L. malefermentans | L. farciminis | Legionella nautarum | Legionella spiritensis | |
| Legionella | L. fermentum | Legionella norrlandica | Legionella steelei | |
| Legionella adelaidensis | L. fornicalis | Legionella oakridgensis | Legionella steigerwaltii | |
| Legionella anisa | L. fructivorans | Legionella parisiensis | Legionella taurinensis | |
| Legionella beliardensis | L. frumenti | Legionella pittsburghensis | Legionella tucsonensis | |
| Legionella birminghamensis | Legionella drancourtii | Legionella pneumophila | Legionella tunisiensis | |
| Legionella bozemanae | Legionella dresdenensis | Legionella quateirensis | Legionella wadsworthii | |
| Legionella brunensis | Legionella drozanskii | Prevotella | Legionella waltersii | |
| Legionella busanensis | Legionella dumoffii | Prevotella albensis | Legionella worsleiensis | |
| Legionella cardiaca | Legionella erythra | Prevotella amnii | Legionella yabuuchiae | |
| Legionella cherrii | Legionella fairfieldensis | Prevotella bergensis | Quadrisphaera | |
| Legionella cincinnatiensis | Legionella fallonii | Prevotella bivia | Quadrisphaera granulorum | |
| Legionella clemsonensis | Legionella feeleii | Prevotella brevis | Quatrionicoccus | |
| Legionella donaldsonii | Legionella geestiana | Prevotella bryantii | Quatrionicoccus australiensis | |
| Oceanibulbus | Legionella genomospecies | Prevotella buccae | Quinella | |
| Oceanibulbus indolifex | Legionella gormanii | Prevotella buccalis | Quinella ovalis | |
| Oceanicaulis | Legionella gratiana | Prevotella copri | Ralstonia | |
| Oceanicaulis alexandrii | Legionella gresilensis | Prevotella dentalis | Ralstonia eutropha | |
| Oceanicola | Legionella hackeliae | Prevotella denticola | Ralstonia insidiosa | |
| Oceanicola batsensis | Legionella impletisoli | Prevotella disiens | Ralstonia mannitolilytica | |
| Oceanicola granulosus | Legionella israelensis | Prevotella histicola | Ralstonia pickettii | |
| Oceanicola nanhaiensis | Legionella jamestowniensis | Prevotella intermedia | Ralstonia pseudosolanacearum | |
| Oceanimonas | Paenibacillus | Prevotella maculosa | Ralstonia syzygii | |
| Oceanimonas baumannii | Paenibacillus thiaminolyticus | Prevotella marshii | Ralstonia solanacearum | |
| Oceaniserpentilla | Pantoea | Prevotella melaninogenica | Ramlibacter | |
| Oceaniserpentilla haliotis | Pantoea agglomerans | Prevotella micans | Ramlibacter henchirensis | |
| Oceanisphaera | Paracoccus | Prevotella multiformis | Ramlibacter tataouinensis | |
| Oceanisphaera donghaensis | Paracoccus alcaliphilus | Prevotella nigrescens | Raoultella | |
| Oceanisphaera litoralis | Paucimonas | Prevotella oralis | Raoultella ornithinolytica | |
| Oceanithermus | Paucimonas lemoignei | Prevotella oris | Raoultella planticola | |
| Oceanithermus desulfurans | Pectobacterium | Prevotella oulorum | Raoultella terrigena | |
| Oceanithermus profundus | Pectobacterium aroidearum | Prevotella pallens | Rathayibacter | |
| Oceanobacillus | Pectobacterium atrosepticum | Prevotella salivae | Rathayibacter caricis | |
| Oceanobacillus caeni | Pectobacterium betavasculorum | Prevotella stercorea | Rathayibacter festucae | |
| Oceanospirillum | Pectobacterium cacticida | Prevotella tannerae | Rathayibacter iranicus | |
| Oceanospirillum linum | Pectobacterium carnegieana | Prevotella timonensis | Rathayibacter rathayi | |
| Saccharococcus | Pectobacterium carotovorum | Prevotella veroralis | Rathayibacter toxicus | |
| Saccharococcus thermophilus | Pectobacterium chrysanthemi | Providencia | Rathayibacter tritici | |
| Saccharomonospora | Pectobacterium cypripedii | Providencia stuartii | Rhodobacter | |
| Saccharomonospora azurea | Pectobacterium rhapontici | Pseudomonas | Rhodobacter sphaeroides | |
| Saccharomonospora cyanea | Pectobacterium wasabiae | Pseudomonas aeruginosa | Ruegeria | |
| Saccharomonospora viridis | Planococcus | Pseudomonas alcaligenes | Ruegeria gelatinovorans | |
| Saccharophagus | Planococcus citreus | Pseudomonas anguillispetica | Stenotrophomonas | |
| Saccharophagus degradans | Planomicrobium | Pseudomonas fluorescens | Stenotrophomonas maltophilia | |
| Saccharopolyspora | Planomicrobium okeanokoites | Pseudoalteromonas haloplanktis | Streptococcus | |
| Saccharopolyspora erythraea | Plesiomonas | Pseudomonas mendocina | [also see below] | |
| Saccharopolyspora gregorii | Plesiomonas shigelloides | Pseudomonas pseudoalcaligenes | Streptomyces | |
| Saccharopolyspora hirsuta | Proteus | Pseudomonas putida | Streptomyces achromogenes | |
| Saccharopolyspora hordei | Proteus vulgaris | Pseudomonas tutzeri | Streptomyces cesalbus | |
| Saccharopolyspora rectivirgula | Sagittula | Pseudomonas syringae | Streptomyces cescaepitosus | |
| Saccharopolyspora spinosa | Sagittula stellata | Psychrobacter | Streptomyces cesdiastaticus | |
| Saccharopolyspora taberi | Salegentibacter | Psychrobacter faecalis | Streptomyces cesexfoliatus | |
| Saccharothrix | Salegentibacter salegens | Psychrobacter phenylpyruvicus | Streptomyces fimbriatus | |
| Saccharothrix australiensis | Salimicrobium | Sanguibacter | Streptomyces fradiae | |
| Saccharothrix coeruleofusca | Salimicrobium album | Sanguibacter keddieii | Streptomyces fulvissimus | |
| Saccharothrix espanaensis | Salinibacter | Sanguibacter suarezii | Streptomyces griseoruber | |
| Saccharothrix longispora | Salinibacter ruber | Saprospira | Streptomyces griseus | |
| Saccharothrix mutabilis | Salinicoccus | Saprospira grandis | Streptomyces lavendulae | |
| Saccharothrix syringae | Salinicoccus alkaliphilus | Sarcina | Streptomyces phaeochromogenes | |
| Saccharothrix tangerinus | Salinicoccus hispanicus | Sarcina maxima | Streptomyces thermodiastaticus | |
| Saccharothrix texasensis | Salinicoccus roseus | Sarcina ventriculi | Streptomyces tubercidicus | |
| Staphylococcus | Salinispora | Sebaldella | S. schleiferi | |
| S. arlettae | Salinispora arenicola | Sebaldella termitidis | S. sciuri | |
| S. agnetis | Salinispora tropica | Serratia | S. simiae | |
| S. aureus | Salinivibrio | Serratia fonticola | S. simulans | |
| S. auricularis | Salinivibrio costicola | Serratia marcescens | S. stepanovicii | |
| S. capitis | Salmonella | Sphaerotilus | S. succinus | |
| S. caprae | Salmonella bongori | Sphaerotilus natans | S. vitulinus | |
| S. carnosus | Salmonella enterica | Sphingobacterium | S. warneri | |
| S. caseolyticus | Salmonella subterranea | Sphingobacterium multivorum | S. xylosus | |
| S. chromogenes | Salmonella typhi | Staphylococcus | Streptococcus thermophilus | |
| S. cohnii | S. equorum | [see below] | Streptococcus sanguinis | |
| S. condimenti | S. felis | S. microti | Streptococcus sobrinus | |
| S. delphini | S. fleurettii | S. muscae | Streptococcus suis | |
| S. devriesei | S. gallinarum | S. nepalensis | Streptococcus uberis | |
| S. epidermidis | S. haemolyticus | S. pasteuri | Streptococcus vestibularis | |
| Streptococcus | S. hominis | S. petrasii | Streptococcus viridans | |
| Streptococcus agalactiae | S. hyicus | S. pettenkoferi | Streptococcus zooepidemicus | |
| Streptococcus anginosus | S. intermedius | S. piscifermentans | Virgibacillus | |
| Streptococcus bovis | S. kloosii | S. pseudintermedius | Virgibacillus halodenitrificans | |
| Streptococcus canis | S. leei | S. pseudolugdunensis | Virgibacillus pantothenticus | |
| Streptococcus constellatus | S. lentus | S. pulvereri | Weissella | |
| Streptococcus downei | S. lugdunensis | S. rostri | Weissella cibaria | |
| Streptococcus dysgalactiae | S. lutrae | S. saccharolyticus | Weissella confusa | |
| Streptococcus equines | S. lyticans | S. saprophyticus | Weissella halotolerans | |
| Streptococcus faecalis | S. massiliensis | Streptococcus orisratti | Weissella hellenica | |
| Streptococcus ferus | Streptococcus infantarius | Streptococcus parasanguinis | Weissella kandleri | |
| Uliginosibacterium | Streptococcus iniae | Streptococcus peroris | Weissella koreensis | |
| Uliginosibacterium gangwonense | Streptococcus intermedius | Streptococcus pneumoniae | Weissella minor | |
| Ulvibacter | Streptococcus lactarius | Streptococcus pseudopneumoniae | Weissella paramesenteroides | |
| Ulvibacter litoralis | Streptococcus milleri | Streptococcus pyogenes | Weissella soli | |
| Umezawaea | Streptococcus mitis | Streptococcus ratti | Weissella thailandensis | |
| Umezawaea tangerina | Streptococcus mutans | Streptococcus salivariu | Weissella viridescens | |
| Undibacterium | Streptococcus oralis | Vibrio | Williamsia | |
| Undibacterium pigrum | Streptococcus tigurinus | Vibrio aerogenes | Williamsia marianensis | |
| Ureaplasma | Vagococcus | Vibrio aestuarianus | Williamsia maris | |
| Ureaplasma urealyticum | Vagococcus carniphilus | Vibrio albensis | Williamsia serinedens | |
| Ureibacillus | Vagococcus elongatus | Vibrio alginolyticus | Winogradskyella | |
| Ureibacillus composti | Vagococcus fessus | Vibrio campbellii | Winogradskyella thalassocola | |
| Ureibacillus suwonensis | Vagococcus fluvialis | Vibrio cholerae | Wolbachia | |
| Ureibacillus terrenus | Vagococcus lutrae | Vibrio cincinnatiensis | Wolbachia persica | |
| Ureibacillus thermophilus | Vagococcus salmoninarum | Vibrio coralliilyticus | Wolinella | |
| Ureibacillus thermosphaericus | Variovorax | Vibrio cyclitrophicus | Wolinella succinogenes | |
| Xenophilus | Variovorax boronicumulans | Vibrio diazotrophicus | Zobellia | |
| Xenophilus azovorans | Variovorax dokdonensis | Vibrio fluvialis | Zobellia galactanivorans | |
| Xenorhabdus | Variovorax paradoxus | Vibrio furnissii | Zobellia uliginosa | |
| Xenorhabdus beddingii | Variovorax soli | Vibrio gazogenes | Zoogloea | |
| Xenorhabdus bovienii | Veillonella | Vibrio halioticoli | Zoogloea ramigera | |
| Xenorhabdus cabanillasii | Veillonella atypica | Vibrio harveyi | Zoogloea resiniphila | |
| Xenorhabdus doucetiae | Veillonella caviae | Vibrio ichthyoenteri | Zooshikella | |
| Xenorhabdus griffiniae | Veillonella criceti | Vibrio mediterranei | Zooshikella ganghwensis | |
| Xenorhabdus hominickii | Veillonella dispar | Vibrio metschnikovii | Zunongwangia | |
| Xenorhabdus koppenhoeferi | Veillonella montpellierensis | Vibrio mytili | Zunongwangia profunda | |
| Xenorhabdus nematophila | Veillonella parvula | Vibrio natriegens | Zymobacter | |
| Xenorhabdus poinarii | Veillonella ratti | Vibrio navarrensis | Zymobacter palmae | |
| Xylanibacter | Veillonella rodentium | Vibrio nereis | Zymomonas | |
| Xylanibacter oryzae | Venenivibrio | Vibrio nigripulchritudo | Zymomonas mobilis | |
| | Venenivibrio stagnispumantis | Vibrio ordalii | Zymophilus | |
| | Verminephrobacter | Vibrio orientalis | Zymophilus paucivorans | |
| | Verminephrobacter eiseniae | Vibrio parahaemolyticus | Zymophilus raffinosivorans | |
| | Verrucomicrobium | Vibrio pectenicida | | |
| | Verrucomicrobium spinosum | Vibrio penaeicida | | |
| | Yangia | Vibrio proteolyticus | | |
| | Yangia pacifica | Vibrio shilonii | | |
| | Yaniella | Vibrio splendidus | | |
| | Yaniella flava | Vibrio tubiashii | | |
| | Yaniella halotolerans | Vibrio vulnificus | | |
| | Yeosuana | Yersinia mollaretii | | |
| | Yeosuana aromativorans | Yersinia philomiragia | | |
| | Yersinia | Yersinia pestis | | |
| | Yersinia aldovae | Yersinia pseudotuberculosis | | |
| | Yersinia bercovieri | Yersinia rohdei | | |
| | Yersinia enterocolitica | Yersinia ruckeri | | |
| | Yersinia entomophaga | Yokenella | | |
| | Yersinia frederiksenii | Yokenella regensburgei | | |
| | Yersinia intermedia | Yonghaparkia | | |
| | Yersinia kristensenii | Yonghaparkia alkaliphila | | |
| | | Zavarzinia | | |
| | | Zavarzinia compransoris | | |

TABLE 3
Medicaments

| | | | |
|---|---|---|---|
| ACE inhibitors with calcium | decongestants | respiratory agents | otic agents |
| channel blocking agents | dermatological agents | sex hormones | renin inhibitors |
| ACE inhibitors with thiazides | diagnostic radiopharmaceuticals | topical agents | respiratory agents |
| adamantane antivirals | diarylquinolines | uncategorized agents | respiratory inhalant products |
| adrenal cortical steroids | dibenzazepine anticonvulsants | vaginal agents | rifamycin derivatives |
| adrenal corticosteroid inhibitors | digestive enzymes | mitotic inhibitors | salicylates |
| adrenergic bronchodilators | dipeptidyl peptidase 4 inhibitors | monoamine oxidase inhibitors | sclerosing agents |
| agents for hypertensive emergencies | diuretics | mouth and throat products | second generation cephalosporins |
| agents for pulmonary hypertension | dopaminergic antiparkinsonism | mTOR inhibitors | selective estrogen receptor |
| aldosterone receptor antagonists | agents | mucolytics | modulators |
| alkylating agents | drugs used in alcohol dependence | multikinase inhibitors | selective immunosuppressants |
| allergenics | echinocandins | muscle relaxants | selective phosphodiesterase-4 |
| alpha-glucosidase inhibitors | EGFR inhibitors | mydriatics | inhibitors |
| alternative medicines | estrogen receptor antagonists | narcotic analgesic combinations | selective serotonin reuptake |
| amebicides | estrogens | narcotic analgesics | inhibitors |
| aminoglycosides | expectorants | nasal anti-infectives | serotonin-norepinephrine reuptake |
| aminopenicillins | factor Xa inhibitors | nasal antihistamines and | inhibitors |
| aminosalicylates | fatty acid derivative anticonvulsants | decongestants | serotoninergic neuroenteric |
| AMPA receptor antagonists | fibric acid derivatives | nasal lubricants and irrigations | modulators |
| amylin analogs | first generation cephalosporins | nasal preparations | sex hormone combinations |
| analgesic combinations | fourth generation cephalosporins | nasal steroids | sex hormones |
| analgesics | functional bowel disorder agents | natural penicillins | SGLT-2 inhibitors |
| androgens and anabolic steroids | gallstone solubilizing agents | neprilysin inhibitors | skeletal muscle relaxant |
| angiotensin converting enzyme | gamma-aminobutyric acid analogs | neuraminidase inhibitors | combinations |
| inhibitors | gamma-aminobutyric acid reuptake | neuromuscular blocking agents | skeletal muscle relaxants |
| angiotensin II inhibitors with | inhibitors | neuronal potassium channel openers | smoking cessation agents |
| calcium channel blockers | gastrointestinal agents | next generation cephalosporins | somatostatin and somatostatin |
| angiotensin II inhibitors with | general anesthetics | nicotinic acid derivatives | analogs |
| thiazides | genitourinary tract agents | NK1 receptor antagonists | spermicides |
| angiotensin receptor blockers | GI stimulants | NNRTIs | statins |
| angiotensin receptor blockers and | glucocorticoids | non-cardioselective beta blockers | sterile irrigating solutions |
| neprilysin inhibitors | glucose elevating agents | non-iodinated contrast media | streptomyces derivatives |
| anorectal preparations | glycopeptide antibiotics | non-ionic iodinated contrast media | succinimide anticonvulsants |
| anorexiants | glycoprotein platelet inhibitors | non-sulfonylureas | sulfonamides |
| antacids | glycylcyclines | nonsteroidal anti-inflammatory | sulfonylureas |
| anthelmintics | gonadotropin releasing hormones | agents | synthetic ovulation stimulants |
| anti-angiogenic ophthalmic agents | gonadotropin-releasing hormone | NS5A inhibitors | tetracyclic antidepressants |
| anti-CTLA-4 monoclonal | antagonists | nucleoside reverse transcriptase | tetracyclines |
| antibodies | gonadotropins | inhibitors (NRTIs) | therapeutic radiopharmaceuticals |
| anti-infectives | group I antiarrhythmics | nutraceutical products | therapeutic vaccines |
| Anti-PD-1 monoclonal antibodies | group II antiarrhythmics | nutritional products | thiazide diuretics |
| antiadrenergic agents (central) with | group III antiarrhythmics | ophthalmic anesthetics | thiazolidinediones |
| thiazides | group IV antiarrhythmics | ophthalmic anti-infectives | thioxanthenes |
| antiadrenergic agents (peripheral) | group V antiarrhythmics | ophthalmic anti-inflammatory | third generation cephalosporins |
| with thiazides | growth hormone receptor blockers | agents | thrombin inhibitors |
| antiadrenergic agents, centrally | growth hormones | ophthalmic antihistamines and | thrombolytics |
| acting | guanylate cyclase-C agonists | decongestants | thyroid drugs |
| antiadrenergic agents, peripherally | H. pylori eradication agents | ophthalmic diagnostic agents | TNF alfa inhibitors |
| acting | H2 antagonists | ophthalmic glaucoma agents | tocolytic agents |
| antiandrogens | hedgehog pathway inhibitors | ophthalmic lubricants and | topical acne agents |
| antianginal agents | hematopoietic stem cell mobilizer | irrigations | topical agents |
| antiarrhythmic agents | heparin antagonists | ophthalmic preparations | topical anesthetics |
| antiasthmatic combinations | heparins | ophthalmic steroids | topical anti-infectives |
| antibiotics/antineoplastics | HER2 inhibitors | ophthalmic steroids with anti- | topical anti-rosacea agents |
| anticholinergic antiemetics | herbal products | infectives | topical antibiotics |
| anticholinergic antiparkinson agents | histone deacetylase inhibitors | ophthalmic surgical agents | topical antifungals |
| anticholinergic bronchodilators | hormones | oral nutritional supplements | topical antihistamines |
| anticholinergic chronotropic agents | hormones/antineoplastics | immunostimulants | topical antineoplastics |
| anticholinergics/antispasmodics | hydantoin anticonvulsants | immunosuppressants | topical antipsoriatics |
| anticoagulant reversal agents | hydrazide derivatives | otic anesthetics | topical antivirals |
| anticoagulants | immune globulins | otic anti-infectives | topical astringents |
| anticonvulsants | immunologic agents | otic preparations | topical debriding agents |
| antidepressants | immunostimulants | otic steroids | topical depigmenting agents |
| antidiabetic agents | immunosuppressive agents | otic steroids with anti-infectives | topical emollients |
| antidiabetic combinations | impotence agents | oxazolidinedione anticonvulsants | topical keratolytics |
| antidiarrheals | in vivo diagnostic biologicals | oxazolidinone antibiotics | topical non-steroidal anti- |
| antidiuretic hormones | incretin mimetics | parathyroid hormone and analogs | inflammatories |
| antidotes | inhaled anti-infectives | PARP inhibitors | topical photochemotherapeutics |
| antiemetic/antivertigo agents | inhaled corticosteroids | PCSK9 inhibitors | topical rubefacient |
| antifungals | inotropic agents | penicillinase resistant penicillins | topical steroids |
| antigonadotropic agents | insulin | penicillins | topical steroids with anti-infectives |
| antigout agents | insulin-like growth factor | peripheral opioid receptor | triazine anticonvulsants |
| antihistamines | integrase strand transfer inhibitor | antagonists | tricyclic antidepressants |
| antihyperlipidemic agents | interferons | peripheral opioid receptor mixed | trifunctional monoclonal antibodies |
| antihyperlipidemic combinations | interleukin inhibitors | agonists/antagonists | ultrasound contrast media |
| antihypertensive combinations | interleukins | peripheral vasodilators | upper respiratory combinations |
| antihyperuricemic agents | intravenous nutritional products | peripherally acting antiobesity | urea anticonvulsants |
| antimalarial agents | iodinated contrast media | agents | urea cycle disorder agents |
| antimalarial combinations | ionic iodinated contrast media | phenothiazine antiemetics | urinary anti-infectives |
| antimalarial quinolines | iron products | phenothiazine antipsychotics | urinary antispasmodics |
| antimetabolites | ketolides | phenylpiperazine antidepressants | urinary pH modifiers |
| antimigraine agents | laxatives | phosphate binders | uterotonic agents |
| antineoplastic detoxifying agents | leprostatics | plasma expanders | vaccine combinations |
| antineoplastic interferons | leukotriene modifiers | platelet aggregation inhibitors | vaginal anti-infectives |
| antineoplastics | lincomycin derivatives | platelet-stimulating agents | vaginal preparations |
| antiparkinson agents | local injectable anesthetics | polyenes | vasodilators |
| antiplatelet agents | local injectable anesthetics with | potassium sparing diuretics with | vasopressin antagonists |
| antipseudomonal penicillins | corticosteroids | thiazides | vasopressors |
| antipsoriatics | loop diuretics | potassium-sparing diuretics | VEGF/VEGFR inhibitors |
| antipsychotics | lung surfactants | probiotics | viral vaccines |
| antirheumatics | lymphatic staining agents | progesterone receptor modulators | viscosupplementation agents |
| antiseptic and germicides | lysosomal enzymes | progestins | vitamin and mineral combinations |
| antithyroid agents | macrolide derivatives | prolactin inhibitors | vitamins |
| antitoxins and antivenins | macrolides | prostaglandin D2 antagonists | 5-alpha-reductase inhibitors |
| antituberculosis agents | magnetic resonance imaging | protease inhibitors | 5-aminosalicylates |
| antituberculosis combinations | contrast media | protease-activated receptor-1 | 5HT3 receptor antagonists |
| antitussives | mast cell stabilizers | antagonists | chloride channel activators |
| antiviral agents | medical gas | proteasome inhibitors | cholesterol absorption inhibitors |
| antiviral boosters | meglitinides | proton pump inhibitors | cholinergic agonists |
| antiviral combinations | metabolic agents | psoralens | cholinergic muscle stimulants |
| antiviral interferons | methylxanthines | psychotherapeutic agents | cholinesterase inhibitors |
| anxiolytics, sedatives, and | mineralocorticoids | psychotherapeutic combinations | CNS stimulants |
| hypnotics | minerals and electrolytes | purine nucleosides | coagulation modifiers |
| aromatase inhibitors | agents | pyrrolidine anticonvulsants | colony stimulating factors |
| atypical antipsychotics | analgesics | quinolones | contraceptives |
| azole antifungals | antibiotics | radiocontrast agents | corticotropin |
| bacterial vaccines | anticonvulsants | radiologic adjuncts | coumarins and indandiones |
| barbiturate anticonvulsants | antidepressants | radiologic agents | cox-2 inhibitors |
| barbiturates | antidiabetic agents | radiologic conjugating agents | |
| BCR-ABL tyrosine kinase | antiemetics | radiopharmaceuticals | |
| inhibitors | antifungals | recombinant human erythropoietins | |
| benzodiazepine anticonvulsants | antihyperlipidemic agents | anticonvulsants | |
| benzodiazepines | antihypertensive combinations | carbonic anhydrase inhibitors | |
| beta blockers with calcium channel | antimalarials | cardiac stressing agents | |
| blockers | antineoplastics | cardioselective beta blockers | |
| beta blockers with thiazides | antiparkinson agents | cardiovascular agents | |
| beta-adrenergic blocking agents | antipsychotic agents | catecholamines | |
| beta-lactamase inhibitors | antituberculosis agents | CD20 monoclonal antibodies | |
| bile acid sequestrants | antivirals | CD30 monoclonal antibodies | |
| biologicals | anxiolytics, sedatives and | CD33 monoclonal antibodies | |
| bisphosphonates | hypnotics | CD38 monoclonal antibodies | |
| bone morphogenetic proteins | bone resorption inhibitors | CD52 monoclonal antibodies | |
| bone resorption inhibitors | cardiovascular agents | central nervous system agents | |
| bronchodilator combinations | central nervous system agents | cephalosporins | |
| bronchodilators | coagulation modifiers | cephalosporins/beta-lactamase | |
| calcimimetics | diagnostic dyes | inhibitors | |
| calcineurin inhibitors | diuretics | cerumenolytics | |
| calcitonin | genitourinary tract agents | CFTR combinations | |
| calcium channel blocking agents | GI agents | CFTR potentiators | |
| carbamate anticonvulsants | hormones | chelating agents | |
| carbapenems | metabolic agents | chemokine receptor antagonist | |
| carbonic anhydrase inhibitors | ophthalmic agents | | |

TABLE 4
Example Conjugation Genes

| Conj. System | VirB | IncN Tra | IncF Tra |
|---|---|---|---|
| T4SS Family | MPFT | MPFN | MPFF |
| Ref. Plasmid | pTI (A. Tumefaciens) | pKM101 | F |
| Transglycosylase - Pilus biogenisis | virB1 | TraL | Orf196(p19) |
| Pilus biogenisis | VirB2 | TraM | TraA |
| Part of IMC | VirB3 | TraA | TraL |
| Major ATPase part of IMC | VirB4 | TraB | TraC |
| Pilus tip adhesin | VirB5 | TraC | TraE |
| Inner membrane channel of IMC | VirB6 | TraD | TraG |
| Outer membrane part of CC | VirB7 | TraN | |
| Channel compnent of inner membrane | VirB8 | TraE | TraH |
| Outer membrane channel of CC | VirB9 | TraO | TraK |
| Membrane spanning channel of CC | VirB10 | TraF | TraB |
| ATPase. Processess ssDNA from T4CP | VirB11 | TraG | |
| T4CP | VirD4 | TraJ | TraD |

1. At least one nucleic acid vector for transfer into a host cell of a microbiota, the vector(s) comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to regulate expression or activity of P1;
wherein
(c) NS1 is under the control of a first promoter (optionally a constitutive or inducible promoter) for expression of P1; and
(d) expression or activity of P2 in the host cell is regulatable by exposure of the host cell comprising the at least one vector to a regulator agent (R), thereby regulating the expression or activity of P1.

2. At least one nucleic acid vector according to claim 1 for transfer into a host cell of a microbiota, the vector(s) comprising nucleic acid that comprises
a) an expressible nucleotide sequence of interest (NS1) for producing a product of interest (P1) in the host cell; and
b) an expressible nucleotide sequence (NS2) for producing a regulator product (P2) in the host cell, wherein P2 is operable in the host cell to bind to vector nucleic acid to regulate expression of P1;
wherein
c) NS1 is under the control of a first promoter (optionally a constitutive or inducible promoter) for expression of P1; and
d) NS2 is under the control of a second promoter that is regulatable for expression of P2, wherein binding or exposure of a regulator agent (R) to the vector nucleic acid regulates the second promoter, thereby regulating the expression of P2 and P1; optionally wherein said at least one vector is one vector that comprises both NS1 and NS2.

3. The vector(s) of claim 1 or 2, wherein
(a) P2 is operable in the host cell to bind to the vector nucleic acid to downregulate expression of P1; and/or
(b) R upregulates expression of P2.

4. The vector(s) of any preceding claim, wherein P2 is expressible in the host cell for the formation of a nuclease that is operable in the host cell to cut the nucleic acid, optionally wherein the nucleic acid is degraded, thereby downregulating the expression of P1.

5. The vector(s) of any preceding claim, wherein P2 comprises
(a) an RNA-guided nuclease;
(b) an RNA that is operable for guiding an RNA-guided nuclease or a precursor of such an RNA; or
(c) a restriction endonuclease.

6. The vector(s) of claim 5, wherein the guided nuclease is a Cas nuclease, TALEN, meganuclease or zinc finger nuclease, preferably a Cas nuclease.

7. The vector(s) of any one of claims 4-6, wherein the nuclease is operable to cut the nucleic acid at a predetermined sequence motif, optionally a protospacer sequence or restriction site.

8. The vector(s) of claim 7, wherein the nucleic acid comprises a plurality of said motifs.

9. The vector(s) of any preceding claim, wherein P1 is an amino acid, protein or RNA for human or animal therapy.

10. The vector(s) of any preceding claim, wherein P1 is toxic to cells of the same species as the host cell.

11. The vector(s) of any preceding claim, wherein P1 is a transcription or translation regulator in cells of the same species as the host cell.

12. The vector(s) of any preceding claim, wherein R is an amino acid, protein, carbohydrate (optionally a sugar), lipid, metal ion or nucleic acid; or wherein R is a sugar alcohol (optionally xylitol).

13. The vector or any preceding claim, wherein the vector is an ICE (integrative and conjugative element), plasmid (optionally a conjugative plasmid), transduction particle (optionally a phage or non-self-replicative transduction particle) or nanoparticle.

14. The vector(s) of claim 13, wherein the vector comprised by a carrier cell, optionally wherein the vector is a conjugative plasmid comprised by a carrier cell (optionally a bacterial carrier cell) for administration to a microbiota of a human or animal subject.

15. The vector(s) of claim 14, wherein the carrier cell is a cell of commensal or probiotic bacterial cell species of a human or animal microbiota and/or the carrier cell is a cell of a human or animal gut microbiota species.

16. A nucleic acid vector (optionally according to claim 14 or 15) for transfer into a host cell of a microbiota, wherein the vector is comprised by a carrier cell and encodes
(a) a nuclease (optionally an RNA-guided nuclease or restriction endonuclease) that is operable in the carrier cell to cut a chromosome or episome (which is not the vector) of the carrier cell, optionally wherein the chromosome or episome is degraded; and/or
(b) an RNA that is operable in the carrier cell for guiding an RNA-guided nuclease or a precursor of such an RNA, wherein the RNA guides the nuclease to cut a chromosome or episome (which is not the vector) of the carrier cell, optionally wherein the chromosome or episome is degraded;
wherein the vector comprises one or more regulatable promoters for regulating expression of the nuclease of (a) and/or the RNA or component of (b) in the carrier cell.

17. The vector of claim 16, wherein the vector comprises an inducible or repressible promoter that regulates expression of the nuclease of (a) and/or the vector comprises an inducible or repressible promoter that regulates expression of the RNA or component of (b), preferably wherein the promoter(s) are inducible promoters.

18. The vector of claim 16 or 17, wherein the guided nuclease is a Cas nuclease, TALEN, meganuclease or zinc finger nuclease, preferably a Cas nuclease.

19. The vector of any one of claims 16-18, wherein the nuclease is the same guided nuclease as recited in claim 5.

20. The vector of any one of claims 16-19, wherein the nuclease is operable to cut the chromosome or episome in the carrier cell at a predetermined sequence motif, optionally a protospacer sequence or restriction site.

21. The vector of any one of claims 16-20, wherein cutting of the carrier cell chromosome or episome kills the carrier cell or reduces growth or proliferation of the carrier cell, preferably wherein the cell is killed.

22. The vector of any one of claim 16-21, wherein the vector comprises an oriT for transfer into the host cell, optionally wherein the vector is a conjugative plasmid.

23. The vector of any one of claims 16-22, wherein
(a) the vector is a conjugative plasmid;
(b) the vector comprises an inducible promoter that regulates expression of the nuclease of (a) and/or the vector comprises an inducible promoter that regulates expression of the RNA or component;
(c) optionally the guided nuclease is a Cas nuclease; and
(d) cutting of the carrier cell chromosome or episome kills the carrier cell or reduces growth or proliferation of the carrier cell.

24. The vector(s) of any preceding claim, wherein the host cell is a cell of a species found in a microbiota (optionally gut microbiota) of humans or animals.

25. The vector(s) of any preceding claim, wherein the host cell is a cell of commensal or probiotic bacterial cell species of a human or animal microbiota.

26. The vector(s) of any one of claims 16 to 25, wherein the species is selected from any species in Table 1, preferably a Bacteroides or Clostridales species.

27. The vector(s) of any preceding claim, wherein
(a) P1 is a protein or RNA for human or animal therapy; and
(b) P2 comprises (i) a crRNA that is operable in the host cell for guiding a Cas nuclease to bind to a protospacer sequence comprised by the nucleic acid for cutting of the protospacer, optionally wherein the nucleic acid is degraded, thereby downregulating the expression of P1, or (ii) a precursor of such an crRNA.

28. A vector or said at least one vector according to any preceding claim for use as a medicament.

29. A host cell comprising nucleic acid that comprises
(a) an expressible nucleotide sequence of interest (NS1) for producing a product of interest (P1) in the host cell; and
(b) an expressible nucleotide sequence (NS2) for producing a regulator product (P2) in the host cell, wherein P2 is operable in the host cell to regulate expression or activity of P1;
wherein
(c) NS1 is under the control of a first promoter (optionally a constitutive or inducible promoter) for expression of P1; and
(d) expression or activity of P2 in the host cell is regulatable by exposure of the host cell comprising the at least one vector to a regulator agent (R), thereby regulating the expression or activity of P1;
wherein the host cell is a bacterial, archaeal or fungal cell.

30. The cell of claim 29, wherein
(a) the nucleic acid is comprised by at least one nucleic acid vector of the cell;
(b) NS1 is comprised by a nucleic acid vector of the cell and NS2 is comprised by a chromosome of the cell;
(c) NS1 is comprised by a chromosome of the cell and NS2 is comprised by a nucleic acid vector of the cell; or
(d) NS1 is comprised by a chromosome of the cell and NS2 is comprised by a chromosome of the cell.

31. The cell of any one of claim 30(a)-(c) wherein the vector or each vector is a conjugative plasmid for transfer to a cell of a microbiota comprised by a human or animal subject.

32. The cell of any one of claims 29-31, wherein the cell is a cell of commensal or probiotic bacterial species of a human or animal microbiota, optionally an E coli cell or a Bacteroides cell.

33. The cell of any one of claims 29-32, wherein NS2 is under the control of a second promoter that is regulatable for expression of P2, wherein binding of a regulator agent (R) to the vector nucleic acid regulates the second promoter, thereby regulating the expression of P2 and P1.

34. The cell of any one of claims 29-33, wherein P2 comprises an RNA-guided nuclease (optionally a Cas nuclease), wherein the nuclease is operable to cut the nucleic acid at a predetermined sequence motif.

35. The cell of claim 34, wherein
(a) the sequence motif is comprised by a chromosome of the cell and the cutting kills the cell; or wherein the sequence motif is comprised by a gene (on a chromosome or vector of the cell) comprising NS1 for production of P1 and the cutting down-regulates the production of P1; or
(b) the sequence motif is comprised by a said vector comprising NS1.

36. The cell of any one of claims 29-35 for treating or preventing a disease or condition in a human or animal subject, wherein the cell is administered to a microbiota (optionally a gut microbiota) of the subject to produce P1 in the subject thereby treating or preventing the disease or condition in the subject.

37. The cell of any one of claims 29-36 comprising a vector according to any one of claims 1-28.

38. A pharmaceutical composition comprising a vector or said at least one vector, or a cell of any preceding claim and a pharmaceutically-acceptable carrier, diluent or excipient, optionally an antacid.

39. A tablet, suppository, pill, capsule, or liquid formulation for administration to the gastrointestinal tract of a human or animal subject, wherein the tablet, suppository, pill, capsule or liquid formulation comprises a vector or said at least one vector, or a cell according to any one of claims 1 to 37.

40. The tablet, pill or capsule of claim 39, wherein the tablet pill, or capsule comprises an enteric coating.

41. The tablet, pill, capsule or liquid formulation of claim 39 or 40 for use as an orally-administered medicament.

42. A method of temporally regulating the production of an expression product in a human or animal subject, the method comprising
(a) administering to a microbiota (optionally a gut microbiota) of the subject at least one vector comprising nucleic acid, wherein the microbiota comprises a host cell and the nucleic acid encodes a product of interest (P1); optionally wherein the administering is oral or topical administration;
(b) allowing transfer of the nucleic acid into the host cell comprised by the microbiota and expression of P1 in the host cell;
(c) after step (b) exposing the microbiota to a regulator agent (R) that upregulates production of an RNA-guided nuclease/guide RNA complex in the host cell that is capable of targeting a protospacer comprised by the nucleic acid, wherein the nuclease cuts the nucleic acid and expression of P1 is rendered non-functional (optionally by degradation of the cut nucleic acid in the cell), wherein the nuclease (or a component thereof) and/or RNA (or a component thereof) is encoded by the nucleic acid.

43. A method of temporally regulating the production of an expression product in a human or animal subject, the method comprising
(a) administering to a microbiota (optionally a gut microbiota) of the subject a vector that encodes a product of interest (P1), said at least one vector wherein said vector(s) encode a product of interest (P1), cell, composition, tablet, suppository, pill, capsule, or liquid formulation according to any one of claims 1-41; optionally wherein the administering is oral or topical administration;
(b) allowing transfer of the nucleic acid into a host cell comprised by the microbiota and expression of P1 in the host cell; and
(c) after step (b) exposing the microbiota to R (such as by administering R to the subject), wherein R regulates the second promoter, thereby regulating the expression of P2 and P1.

44. A method of
(a) treating or preventing a disease or condition in a human or animal subject by temporally regulating the production of P1 according to the method claim 42 or 43; or
(b) modifying a microbiota (eg, a gut microbiota) of a human or animal subject by temporally regulating the production of P1 according to the method claim 42 or 43.

45. The method of claim 42, 43 or 44, wherein P1 is a therapeutically or prophylactically useful expression product in the subject.

46. The method of any one of claims 42-45, wherein in step (c) P1 expressed from the nucleic acid is the regulator agent (R) or is a component of a pathway that produces R, whereby a P1 expression feedback loop negatively regulates further expression of P1.

47. The method of any one of claims 42-45, wherein R upregulates the second promoter in step (c) and P2 downregulates the expression of P1; and optionally wherein the upregulation of the second promoter causes the production of a guided nuclease or restriction endonuclease that cuts the nucleic acid in the host cell, wherein the nucleic acid is degraded, thereby downregulating the expression of P1.

48. The method of any one of claims 42-45, wherein P2 is capable of upregulating the expression of P1 in the host cell, wherein R downregulates the second promoter in step (c) whereby the expression of P1 is downregulated.

49. The vector, said at least one vector, cell, composition, tablet, suppository, pill, capsule, or liquid formulation according to any one of claims 1-41 for use in the method of any one of claims 42-48, optionally the method of claim 44(a).

50. A cell (optionally according to any one of claims 29-37), comprising a nucleic acid, wherein the nucleic acid comprises a gene encoding a product of interest (P1), the gene comprising a nucleotide sequence (NS1) encoding P1 and a regulatory region 5′ of NS1 that comprises a promoter (Px) for controlling the expression of NS1, wherein the combination of Px and NS1 is heterologous to the cell and Px is regulatable by xylitol or xylose.

51. The cell of claim 50, wherein the promoter is a xylitol or xylose regulatable promoter of a Morganella species, optionally M morganii.

52. The cell of claim 50 or 51, wherein the promoter comprises SEQ ID NO: 3 or a nucleotide sequence that is at least 70% identical to SEQ ID NO: 3.

53. The cell of any one of claims 50-52, wherein Px is homologous to a xylitol or xylose regulatable promoter of Morganella morganii.

54. The cell of any one of claims 50-53, wherein the cell genome encodes a repressor that is capable of repressing Px, wherein xylitol and/or xylose is capable of de-repressing the repressor.

55. The cell of claim 54, wherein the repressor is encoded by SEQ ID NO: 1 or a nucleotide sequence that is at least 70% identical to SEQ ID NO: 1.

56. The cell of any one of claims 50-55, wherein the cell comprises a xylitol transporter, optionally a xylitol ABC transporter.

57. The cell of any one of claims 50-56, wherein the cell is devoid of a xylitol isomerase gene.

58. A nucleic acid vector comprising a gene as recited in any one of claims 50-57.

59. The vector of claim 58, wherein the vector is a plasmid (optionally a conjugative plasmid), transposon, phagemid or a phage.


SEQUENCE LISTING (16 sequences)

SEQ ID NO: 1 - DNA, 1002 bp, Morganella morganii
  source 1..1002: mol_type = genomic DNA
ttggcggaaccccggacacagaaagtcacactggaaaatgtggccgctattgcgaatgtcagtaagatcaccgcatcccgcgcattttcgcagccggataaggttcacccggaaactctcaggcgtattctcgatgccgcagataaaatcggttatgtggtgaatgcggcggcacgcagcctgcgcgcgaaatcctcccgtaccatcggtattgtcagcccggacatgagcaacccgtttttcggcgggctggccaagcgcatcaccctggaagcctataacgccgggtatgacacgctgatgtttgactcctatgaatcacgtgaaaatgaagcgcgtatcattgataagctgattggttacaacgtggatgccatcattctgtccgttgtctccgccgaaagggtgtatcgcccggcgtatatgaaacagctggaactgctcaatattccggtgatcctggttgaccgtgagctggatgccaaggcatgcagcggcgtttacatcgataatctcgactgcggattgcaggcagggcgctatctgctgtcacaaaaagcggataacgtggtgattgtctccggcccggaagattccaatgtggcccaggatcgcgtaaccggcatggtggccggtctgcacggccaggtcagcagcgtcaatgtgctgcatgcggacttcctgatggatgaagcctttaaagtcaccgaccattacctgaaataccatcccgcgccggattattttgtcggctgcaataaccagatctcactcgggattatcaaagcctgtatccgccacaatctgatcccgcaaaaagatgtctcgctgttcagcatcgatgaagtctctcatgccgatatttacggattcaatttcccgtgcatttctcatgatttacaggaaattgcctggcaggccattaacatggcagtacggcgggcgacggatcgcagcgccccggcgagcaaggtggttgtgcgcggtctcctcaaatcttaa

SEQ ID NO: 2 - protein, 333 aa, Morganella morganii
MAEPRTQKVTLENVAAIANVSKITASRAFSQPDKVHPETLRRILDAADKIGYVVNAAARSLRAKSSRTIGIVSPDMSNPFFGGLAKRITLEAYNAGYDTLMFDSYESRENEARIIDKLIGYNVDAIILSVVSAERVYRPAYMKQLELLNIPVILVDRELDAKACSGVYIDNLDCGLQAGRYLLSQKADNVVIVSGPEDSNVAQDRVTGMVAGLHGQVSSVNVLHADFLMDEAFKVTDHYLKYHPAPDYFVGCNNQISLGIIKACIRHNLIPQKDVSLFSIDEVSHADIYGFNFPCISHDLQEIAWQAINMAVRRATDRSAPASKVVVRGLLKS

SEQ ID NO: 3 - DNA, 201 bp, Morganella morganii
  source 1..201: mol_type = genomic DNA
cctgaatgatatcgttatcattatagccttgtcacagatagcaaagatgtgacccggaagaccctttgccgaatactacatgagtagatcagactttatttaataaaagtttaaccatcatcacataattataataaatattgctctattatccgcatcatagtaatgatatcgatatcattttaatgaaaggaaatgatc

SEQ ID NO: 4 - DNA, 1871 bp, Morganella morganii
  source 1..1871: mol_type = genomic DNA
ttaagatttgaggagaccgcgcacaaccaccttgctcgccggggcgctgcgatccgtcgcccgccgtactgccatgttaatggcctgccaggcaatttcctgtaaatcatgagaaatgcacgggaaattgaatccgtaaatatcggcatgagagacttcatcgatgctgaacagcgagacatctttttgcgggatcagattgtggcggatacaggctttgataatcccgagtgagatctggttattgcagccgacaaaataatccggcgcgggatggtatttcaggtaatggtcggtgactttaaaggcttcatccatcaggaagtccgcatgcagcacattgacgctgctgacctggccgtgcagaccggccaccatgccggttacgcgatcctgggccacattggaatcttccgggccggagacaatcaccacgttatccgctttttgtgacagcagatagcgccctgcctgcaatccgcagtcgagattatcgatgtaaacgccgctgcatgccttggcatccagctcacggtcaaccaggatcaccggaatattgagcagttccagctgtttcatatacgccgggcgatacaccctttcggcggagacaacggacagaatgatggcatccacgttgtaaccaatcagcttatcaatgatacgcgcttcattttcacgtgattcataggagtcaaacatcagcgtgtcatacccggcgttataggcttccagggtgatgcgcttggccagcccgccgaaaaacgggttgctcatgtccgggctgacaataccgatggtacgggaggatttcgcgcgcaggctgcgtgccgccgcattcaccacataaccgattttatctgcggcatcgagaatacgcctgagagtttccgggtgaaccttatccggctgcgaaaatgcgcgggatgcggtgatcttactgacattcgcaatagcggccacattttccagtgtgactttctgtgtccggggttccgccaattttctctccttagcagcaacgtatcattttataccattataatatcatctaaatcgattcagtcacgtgaaaaacacgctttttattacttaaaccgacactaacaactttttaacactcgagtctttttatccgcggatcggtaaaaatgtcgtattcatcggtgttttctgtggagaactccgacaccaccgcgccctgcggtccggctttaaaccagtggcgggtatccggcatgatggtgtactgctctcccgggcgcagcagaataaagcggtcacaggtgtaccattcctcatccccctgcggcggacggcagaccggattgccctgagcatcctgattcagtgtcagcgccggatcatccacaaacagatacacctcaccccagcggcagcggaaagtttcctgcttacccggccggtctttgtacggcggatgacggtgctccggacaggtctggtcagcaaagagaaccagctctttggcgcagtacagcgggctgttgcagtacgtgagcagttgcaggccagagcggggataatccggcagattaaagtgagcaatttcaatatgctgctgttccgcttctgtcagaatgatatgtgcctgttccagaaccgccagtgattccgcgatatacggctgtttattcatctgcctctctcctgaatgatatcgttatcattatagccttgtcacagatagcaaagatgtgacccggaagaccctttgccgaatactacatgagtagatcagactttatttaataaaagtttaaccatcatcacataattataataaatattgctctattatccgcatcatagtaatgatatcgatatcattttaatgaaaggaaatgatc

SEQ ID NO: 5 - DNA, 29 bp, synthetic construct
  source 1..29: mol_type = other DNA
gttttatattaactaagtggtatgtaaat

SEQ ID NO: 6 - DNA, 37 bp, synthetic construct
  source 1..37: mol_type = other DNA
ttccggggaactgtaccgggacaactggatagggaaa

SEQ ID NO: 7 - DNA, 37 bp, synthetic construct
  source 1..37: mol_type = other DNA
tgttgtgcaaaaaagcggttagctccttcggtcctcc

SEQ ID NO: 8 - DNA, 37 bp, synthetic construct
  source 1..37: mol_type = other DNA
aactatcaaatagtaaatgatgatatactgaaattta

SEQ ID NO: 9 - DNA, 37 bp, synthetic construct
  source 1..37: mol_type = other DNA
tcaaaaccaatgtaatattccgtccatccggtctgcc

SEQ ID NO: 10 - DNA, 37 bp, synthetic construct
  source 1..37: mol_type = other DNA
gttctattaggtgttacatgctgttcatctgttacat

SEQ ID NO: 11 - DNA, 23 bp, synthetic construct
  source 1..23: mol_type = other DNA
gctagcagagtttgtagaaacgc

SEQ ID NO: 12 - DNA, 22 bp, synthetic construct
  source 1..22: mol_type = other DNA
atgcgtaaaggcgaagaactgt

SEQ ID NO: 13 - DNA, 44 bp, synthetic construct
  source 1..44: mol_type = other DNA
tttctacaaactctgctagcttaagatttgaggagaccgcgcac

SEQ ID NO: 14 - DNA, 54 bp, synthetic construct
  source 1..54: mol_type = other DNA
agttcttcgcctttacgcatgatcatttcctttcattaaaatgatatcgatatc

SEQ ID NO: 15 - DNA, 43 bp, synthetic construct
  source 1..43: mol_type = other DNA
gacctccgggtattctgatgcattgaattcctcctgctagcct

SEQ ID NO: 16 - DNA, 43 bp, synthetic construct
  source 1..43: mol_type = other DNA
actggatgcccgtaaataaatgttgtttgtcggtgaactctgc